(19) United States
(12) Patent Application Publication
(10) Pub. No.: US 2026/0132217 A1
(43) Pub. Date: May 14, 2026

(54) ANTI-sCD146 ANTIBODIES AND USES THEREOF

(71) Applicants: UNIVERSITE D'AIX-MARSEILLE, MARSEILLE (FR); INSTITUT NATIONAL DE LA SANTÉ ET DE LA RECHERCHE MÉDICALE, PARIS (FR); ASSISTANCE PUBLIQUE - HÔPITAUX DE MARSEILLE, MARSEILLE (FR); INSTITUT NATIONAL DE RECHERCHE POUR L'AGRICULTURE, L'ALIMENTATION ET L'ENVIRONNEMENT, PARIS (FR)

(72) Inventors: AHMAD JOSHKON, MARSEILLE (FR); MARCEL BLOT-CHABAUD, FUVEAU (FR); NATHALIE BARDIN, MARSEILLE (FR); BENJAMIN GUILLET, AIX EN PROVENCE (FR)

(21) Appl. No.: 19/119,569

(22) PCT Filed: Oct. 10, 2023
(86) PCT No.: PCT/EP2023/077960

§ 371 Date: Apr. 9, 2025

(30) Foreign Application Priority Data
Oct. 10, 2022 (EP) 22306521.0

(51) Int. Cl. C07K 16/30; A61K 38/00; A61P 35/00; C07K 16/22; C12N 15/62; C12N 15/63; G01N 33/68

(52) U.S. Cl. CPC C07K 16/3092; A61K 38/00; A61P 35/00; C07K 16/22; C12N 15/62; C12N 15/63; G01N 33/6893; C07K 2317/24; C07K 2317/31; C07K 2317/565; G01N 2333/70503

(57) ABSTRACT

The present invention relates to the field of medicine. It more particularly relates to new anti-soluble CD146 (sCD146) protein antibodies, in particular multispecific antibodies comprising antigen binding regions that specifically bind sCD146 and VEGF proteins, to nucleic acids and vectors encoding and/or expressing such antibodies, and to cells and compositions comprising such product(s), as well as to uses thereof, typically in the treatment of cancer, preferably cancer resistant to treatments involving an anti-VEGF antibody. The invention also relates to uses of anti-sCD146 antibodies for predicting or monitoring the response of a subject to a treatment of cancer involving an anti-VEGF agent, and to related methods.

### FIELD OF THE INVENTION

The present invention relates to the field of medicine, in particular of oncology. It more particularly relates to new anti-soluble CD146 (sCD146) protein antibodies, in particular multispecific antibodies comprising antigen binding regions that specifically bind sCD146 and VEGF proteins, to nucleic acids and vectors encoding and/or expressing such antibodies, and to cells and compositions comprising such product(s), as well as to uses thereof, typically in the treatment of diseases, in particular cancer, preferably cancer resistant to treatments involving an anti-VEGF antibody such as bevacizumab. The invention also relates to uses of anti-sCD146 antibodies for predicting or monitoring the response of a subject to a treatment of cancer involving an anti-VEGF agent, and to related methods.

### BACKGROUND OF THE INVENTION

Cancer is a major leading cause of death in the world. Current major treatments for cancer management include surgery, cytotoxic chemotherapy, targeted therapy, radiation therapy, endocrine therapy and immunotherapy.

Angiogenesis, the formation of new blood vessels from preexisting one, represents a critical process for oxygen and nutrient supply to proliferating cells, therefore promoting tumor growth and metastasis. The Vascular Endothelial Growth Factor (VEGF) pathway is one of the key mediators of angiogenesis in cancer. Therefore, several therapies including monoclonal antibodies or tyrosine kinase inhibitors target this axis. Although preclinical studies demonstrated strong antitumor activity, clinical studies were disappointing. Antiangiogenic drugs, used to treat metastatic patients suffering of different types of cancers, prolonged survival to different extents but are not curative.

Resistance to anti-VEGF therapy often occurs owing to the escape mechanisms of the angiogenic process through the activation of signaling pathways other than the VEGF pathway.

Despite the endeavours and achievements made in treating cancers during the past decades, resistance to classical chemotherapeutic agents and/or novel targeted drugs, occurring through many different mechanisms, continues to be a major problem in cancer therapies. Drug resistance, either existing before treatment (intrinsic) or generated after therapy (acquired), is responsible for most relapses of cancer, one of the major causes of death of the disease. Heterogeneity among patients and tumors, and the versatility of cancer to circumvent therapies make drug resistance more challenging to deal with.

Thus, there is a great and urgent need for methods allowing the prediction or efficient monitoring of the response of patients to cancer treatments, in particular those involving an anti-VEGF agent, as well as for methods allowing appropriate treatment of cancer, or subject suffering of cancer, identified as resistant to cancer therapy, and in particular for new and more effective therapeutic agents.

Bispecific antibodies have been developed with the aim of overcoming such resistance mechanisms. However, as recently reported by Wang et al. (“The state of the art of bispecific antibodies for treating human malignancies”, 2021), the use of bi-specific antibodies with the objective of simultaneously blocking two targets in order to overcome the phenomenon of resistance to treatment by compensatory mechanism requires a careful design of the antibody to obtain a good balance of affinities for each respective target as well as the right spatial distance to obtain a sufficient bivalent interaction.

Inventors now herein advantageously provide new therapeutic tools, including new anti-sCD146 antibodies, in particular therapeutically efficient bispecific antibodies, as well as uses and methods involving anti-sCD146 antibodies allowing efficient cancer treatment monitoring and early detection of cancer resistance to treatments.

### SUMMARY OF THE INVENTION

The inventors have developed and herein describe novel antibodies. They have shown that an increase in sCD146 concentration in sera of glioblastoma multiforme (GBM) patients after the first cycle of bevacizumab treatment was significantly associated with poor progression free survival (PFS) and shorter overall survival (OS). These effects were blocked by mucizumab, a novel humanized anti-sCD146 antibody. In vivo, the combination of bevacizumab with mucizumab impeded CD146+ glioblastoma growth and reduced tumor cell dissemination to an extent significantly higher than that observed with bevacizumab alone or mucizumab alone. They also developed a novel bispecific antibody comprising variable regions from mucizumab and bevacizuman which is also able to impeded CD146+ glioblastoma growth in vivo to an extent significantly higher than that observed with bevacizumab alone or mucizumab alone. Thus, the present invention identifies sCD146 as an easily detectable and early non-invasive biomarker to predict, and an efficient target to prevent, bevacizumab resistance in patients with glioblastoma. Inventors herein offer a new targeted therapy combining bevacizumab and mucizumab, either as a composition or as a bispecific antibody, which enhances therapeutic benefits in cancer patients, in particular in patients suffering of a glioblastoma.

In a first aspect, the present description relates to an antibody comprising an antigen binding region that specifically binds a soluble CD146 (sCD146) protein. Said antibody comprises a light chain variable region (VL) comprising the sequence of SEQ ID NO: 1: EIVLTQSPDFQSVTPKEKVTITCRASQGISTSIYWYQQKPDQSPKLLIKFASQSISGVPSRF SGSGSGTDFTLTINRVEAEDAATYYCQQSYNLPYTFGAGTKLEIK, and a heavy chain variable region (VH) comprising the sequence of SEQ ID NO: 2: QVKLVESGGGVVQPGRSLTLSCAASGFTFSDYGMHWIRQAPGKGLEWIAMIYYDSSK MYYADTVKGRFTISRDNSKNTLYLQMNSLRAEDTAVYYCAAFQFDYWGQGTMVTVS S.

In a second aspect, the present description relates to a multispecific antibody, preferably a bispecific antibody, comprising an antigen binding region that specifically binds a sCD146 protein and an antigen binding region that specifically binds a VEGF protein. This multispecific antibody is advantageously capable of simultaneously binding the sCD146 and VEGF proteins.

A particular multispecific antibody comprises an antigen binding region that specifically binds a VEGF protein and which comprises a light chain variable region (L) comprising one or more of the following CDRs:
H-CDR1: GYTFNYFMN (SEQ ID NO: 23) or a variant thereof,
H-CDR2: WINTYTGEPTYAADFKR (SEQ ID NO: 24) or a variant thereof, and
H-CDR3: YPHYYGSSHWYFDV (SEQ ID NO: 25) or a variant thereof,
   and/or a heavy chain variable region (H) comprising one or more of the following CDRs:
L-CDR1: SASQDISNYLN (SEQ ID NO: 26) or a variant thereof,
L-CDR2: FTSSLHS (SEQ ID NO: 27) or a variant thereof, and
L-CDR3: QQYSTVPWT (SEQ ID NO: 28) or a variant thereof,
   said variants having at least 80% sequence identity to the recited CDR sequences, preferably having one, two or three amino acid variations from the recited CDR sequences.

In a preferred aspect, said multipecific antibody, comprises a light chain variable region (VL) comprising sequence SEQ ID NO: 37: DIQMTQSPSSLSASVGDRVTITCSASQDISNYLNWYQQKPGKAPKVLIYFTSSLHSGVPS RFSGSGSGTDFTLTISSLQPEDFATYYCQQYSTVPWTFGQGTKVEIKR, or a variant thereof, and a heavy chain variable region (VH) comprising sequence SEQ ID NO: 38: EVQLVESGGGLVQPGGSLRLSCAASGYTFTNYGMNWVRQAPGKGLEWVGWINTYTG EPTYAADFKRRFTFSLDTSKSTAYLQMNSLRAEDTAVYYCAKYPHYYGSSHWYFDVW GQGTLVTVSS, or a variant thereof, said variants having at least 80% sequence identity to the recited sequences, preferably having one, two or three amino acid variations from the recited sequences.

Another particular multispecific antibody comprises an antigen binding region that specifically binds a sCD146 protein and which comprises a light chain variable region (L) comprising one or more of the following CDRs:
H-CDR1: DYGMH (SEQ ID NO: 3) or a variant thereof,
H-CDR2: MIYYDSSKMYYADTVKG (SEQ ID NO: 4) or a variant thereof, and
H-CDR3: FQFDY (SEQ ID NO: 5) or a variant thereof,
   and/or a heavy chain variable region (H) comprising one or more of the following CDRs:
L-CDR1: ASQGISTSIY (SEQ ID NO: 6) or a variant thereof,
L-CDR2: FASQSIS (SEQ ID NO: 7) or a variant thereof, and
L-CDR3: QQSYNLPYT (SEQ ID NO: 8) or a variant thereof
   said variants having at least 80% sequence identity to the recited CDR sequences, preferably having one, two or three amino acid variations from the recited CDR sequences.

In a preferred aspect, said multipecific antibody, comprises a light chain variable region (VL) comprising the sequence of SEQ ID NO: 1 or a variant thereof, and a heavy chain variable region (VH) comprising the sequence of SEQ ID NO: 2 or a variant thereof, said variants having at least 80% sequence identity to the recited sequences, preferably having one, two or three amino acid variations from the recited sequences

Also herein described are multispecific antibodies comprising several or each of the herein above described CDR sequences and/or variables sequences, i.e., several or each of SEQ ID NO: 1-8, 23-28 and 37-38.

A preferred multispecific antibody, in particular bispecific antibody, according to the invention comprises an antigen binding region that specifically binds a sCD146 protein and an antigen binding region that specifically binds a VEGF protein, and which comprises a first light (L) and a first heavy (H) chain variable region directed against a sCD146 protein, and a second light (L) and a second heavy (H) chain variable region directed against a VEGF protein, wherein the first light (L) chain variable regions directed against a sCD146 protein comprise one or more of the following CDRs:
H-CDR1 of SEQ ID NO: 3 or a variant thereof,
H-CDR2 of SEQ ID NO: 4 or a variant thereof, and
H-CDR3 of SEQ ID NO: 5 or a variant thereof,
   and/or the first heavy (H) chain variable region comprising one or more of the following CDRs:
L-CDR1 of SEQ ID NO: 6 or a variant thereof,
L-CDR2 of SEQ ID NO: 7 or a variant thereof, and
L-CDR3 of SEQ ID NO: 8 or a variant thereof,
   and wherein the second light (L) chain variable region directed against a VEGF protein comprise one or more of the following CDRs:
H-CDR1 of SEQ ID NO: 23 or a variant thereof,
H-CDR2 of SEQ ID NO: 24 or a variant thereof, and
H-CDR3 of SEQ ID NO: 25 or a variant thereof,
   and/or the second heavy (H) chain variable region comprising one or more of the following CDRs:
L-CDR1 of SEQ ID NO: 26 or a variant thereof,
L-CDR2 of SEQ ID NO: 27 or a variant thereof, and
L-CDR3 of SEQ ID NO: 28 or a variant thereof,
   said variants having at least 80% sequence identity to the recited CDR sequences, preferably having one, two or three amino acid variations from the recited CDR sequences.

In a preferred aspect, said multipecific antibody, comprises:
a first light chain variable region comprising, or consisting of, the sequence of SEQ ID NO: 1,
a first heavy chain variable region comprising, or consisting of, the sequence of SEQ ID NO: 2,
a second light chain variable region comprising, or consisting of, the sequence of SEQ ID NO: 37, and
a second heavy chain variable region comprising or consisting of, the sequence of SEQ ID NO: 38.

In another aspect, the description provides a nucleic acid molecule or set of nucleic acid molecules encoding:
i) an antibody comprising an antigen binding region that specifically binds a soluble CD146 protein (sCD146), wherein said antibody comprises a light chain variable region (VL) comprising the sequence of SEQ ID NO: 1 and a heavy chain variable region (VH) comprising the sequence of SEQ ID NO: 2, such as mucizumab,
ii) a multispecific antibody as herein described, or
iii) a combination of a) an antibody as defined in i), an antibody comprising one or more of the CDRs, VL and/or VH sequences specifically recognizing a sCD146 protein (i.e., comprising an antigen binding region that specifically binds a sCD146 protein) as herein described, or variant(s) thereof, with b) an antibody comprising one or more of the CDRs, VL and/or VH sequences specifically recognizing a VEGF protein (i.e., comprising an antigen binding region that specifically binds a VEGF protein) as herein described or variant(s) thereof, or bevacizumab antibody; or sequence(s) complementary to said encoding sequence(s).

In another aspect, the inventors herein provide a vector and a (host) cell comprising a nucleic acid molecule or a set of nucleic acid molecules as herein described.

Also herein described is a composition, typically a pharmaceutical composition, comprising i) a first antibody comprising an antigen binding region that specifically binds a sCD146 protein and second antibody comprising an antigen binding region that specifically binds a VEGF protein, wherein the second antibody comprises SEQ ID NO: 23 (H-CDR1), SEQ ID NO: 24 (H-CDR2), SEQ ID NO: 25 (H-CDR3), SEQ ID NO: 26 (L-CDR1), SEQ ID NO: 27 (L-CDR2) and SEQ ID NO: 28 (L-CDR3), or ii) a multispecific antibody, a nucleic acid molecule or set of nucleic acids, a vector and/or a (host) cell as herein described, and a pharmaceutically acceptable excipient or support.

These biological tools or products (antibodies, nucleic acid molecules, vectors, cells and compositions comprising the same) can be used as medicaments.

The antibody herein described, including the multispecific antibodies, the nucleic acid molecule or set of nucleic acids, the vector, the (host) cell, as well as the pharmaceutical composition, are also herein described for use for preventing or treating cancer. They are also herein described for use for preventing or treating any condition wherein the ill subject does not respond (i.e., is resistant) to treatments involving an anti-VEGF compound or any condition (/pathology) for which resistance to treatment can be shown by an increase in the level of sCD146 in a fluid biological sample of the subject. The condition is typically a cancer, preferably a cancer the conventional treatment of which involves an anti-VEGF compound such as bevacizumab, for example glioblastoma, typically a glioblastoma resistant to anti-VEGF treatment or a glioblastoma whose cells express membranous CD146 (i.e., a CD146+ glioblastoma).

The antibody herein described, including the multispecific antibodies, the nucleic acid molecule or set of nucleic acids, the vector, the (host) cell, as well as the pharmaceutical composition, are in particular herein described for use for treating a subject suffering of cancer who has been, who is, or who will be treated with bevacizumab, or for treating a subject identified as, or known to be, resistant to bevacizumab treatment.

In another aspect, inventors herein describe the use (in particular in vitro or ex vivo) of a soluble CD146 protein as a biomarker for predicting or monitoring the response to a bevacizumab treatment of a subject suffering from cancer.

They describe in particular an in vitro or ex vivo method for monitoring the response to a bevacizumab treatment of a subject suffering from cancer comprising determining the soluble CD146 protein level of expression in a biological sample of said subject at two or more time points during said bevacizumab treatment, wherein a higher soluble CD146 protein level of expression in a biological sample of the subject at a later time point, compared to a reference value obtained in a biological sample of the subject at an earlier time point, is indicative of a resistance of the subject to said bevacizumab treatment whereas an equal or lower soluble CD146 protein level is indicative of a response of the subject to said bevacizumab treatment.

They also describe an in vitro or ex vivo method for predicting the response to a bevacizumab treatment of a subject suffering from cancer comprising i) determining the soluble CD146 protein expression level in a biological sample of the subject before bevacizumab treatment, ii) comparing the expression level determined at step i) with a reference value, and iii) concluding that the subject is likely to respond to bevacizumab treatment when the level determined at step i) is lower than the reference value, or concluding that the patient is unlikely to respond to bevacizumab treatment when the level determined at step i) is higher than the reference value.

### BRIEF DESCRIPTION OF THE DRAWINGS

FIG. 1: sCD146 plasma concentration in patients under bevacizumab treatment.

(A) Patients' characterization. (B) Evolution of sCD146 plasma concentration at baseline versus after one cycle of bevacizumab treatment, in responder and non-responder patients. (C) Progression-Free survival (left) and Overall survival (right) according to sCD146 plasma concentration in patients under bevacizumab treatment.

*p<0.05, non-responders vs responders.

FIG. 2: Bevacizumab induces proliferation and Cancer Stem Cells (CSC)/Epithelial-Mesenchymal Transition (EMT) markers in U87 cells.

In-vitro challenging of U87 cells with bevacizumab (Avastin; 100 μg/ml) for 2 months enhanced cell proliferation (A) and sCD146 secretion (B), normalized to total cell number, as compared to IgG treated cells. Avastin-challenged U87 cells upregulated CD146, VEGFR2 and integrin subunits αv and β3 gene transcription (C) and their membrane expression, except for CD146 (D). U87 challenging with bevacizumab (Avastin) potently induced expression of markers related to CSC (E) and EMT (F) at the mRNA level as compared to control IgG treated cells. Average of 5 experiments is shown; **P<0.01, ***p<0.001, experimental vs control.

FIG. 3: sCD146 induces U87 cell proliferation, migration and invasion and promotes CSC/EMT markers.

U87 cells were treated with 100 ng/ml of VEGF, rsCD146 or combination of both molecules for 48 h and cell proliferation (A), migration (B) and invasion (C) were determined. EMT (D) and CSC (E) markers were also examined after 48 h of treatment with 100 ng/ml sCD146. Representative blots from 5 experiments are shown. *P<0.05, **P<0.01, ***p<0.001, experimental vs control.

FIG. 4: Humanized anti-sCD146 antibody mucizumab significantly decreases U87 cell proliferation, migration and invasion and hampers CSC and EMT in vitro.

U87 cells were treated with conditioned media (CM) containing Irrelevant IgG, bevacizumab (Avastin), Mucizumab, or combination of both antibodies for proliferation (A), migration (B), and invasion assays (C). EMT and CSC markers were also examined at the mRNA (D and E) and protein (F and G) levels. Average of 3 experiments is shown; *P<0.05, **P<0.01, ***p<0.001, experimental vs control.

FIG. 5: Humanized anti-sCD146 antibody mucizumab, significantly reduces tumor growth in two different in vivo models and displays complementary effects with bevacizumab.

Tumor volume was measured in athymic nude mice subcutaneously injected with U87 cells after treatment with IgG, bevacizumab (Avastin), mucizumab or bevacizumab+mucizumab. Representative images of tumors and their weight are shown (A). Mice were orthotopically injected with U87 cells and treated with IgG, Avastin, mucizumab or bevacizumab+mucizumab. Tumor volume was estimated based on immunohistochemical staining of human CD146 in serially cut mice brain sections (B). Tumor cell dissemination across all the brain was also determined. Red arrows show disseminated cells (C). Expression of human specific CD146 and MAX proteins in total brain lysate was determined in each group (D). Imaging was performed in 6 representative mice orthotopically bearing U87 tumors using PET-scan after 86Ga-RGD injection. Results were expressed as ratio of radioactivity between right (RH) and left (LH) hemispheres (E). 10 mice were used in each group. Representative images are shown, *P<0.05, **P<0.01, ***p<0.001, experimental vs control.

FIG. 6: Concentration of VEGF and sCD146 in the culture media of U87, U373, and U118 glioblastoma cell lines. ELISA was performed using cultured media from the three glioblastoma cell lines. The concentrations are normalized to total cell number. Average of 3 experiments is shown, each point was run in triplicate.

FIG. 7: Avastin induces proliferation, sCD146 secretion, and EMT/CSC markers in U373 cells. In vitro challenging of U373 cells with Avastin (100 μg/mL) enhances cell proliferation (A) and increases sCD146 secretion (B) as compared to IgG treated cells. Avastin-challenged U373 cells upregulate CD146, VEGFR2 and integrin subunits αv and β3 gene transcription (C) and their membrane expression, except for CD146 (D) as revealed by q-PCR and flow cytometry, respectively. U373 challenging with Avastin potently induces expression of markers related to CSC (E) and EMT (F) at the mRNA level as compared to control IgG treated cells. Average of 5 experiments is shown; **P<0.01, ***p<0.001, experimental vs control.

FIG. 8: Avastin has no effect on CD146-negative U118 glioblastoma cells. The effect of in vitro challenging of U118 cells with Avastin (100 μg/mL) on cell proliferation (A) and sCD146 secretion by the cells (B) as compared to IgG treated cells are shown. Gene expression of CD146, VEGFR2 and integrin subunits αv and β3 in the Avastin-challenged U118 cells were assessed by q-PCR (C) and their membrane expression by flow cytometry (D). The expression of markers related to Cancer Stem Cells (CSC) (E) and Epithelial-Mesenchymal Transition (EMT) (F) at the mRNA level in Avastin-challenged U118 cells is shown. Average of 3 experiments; ***p<0.001, experimental vs control.

FIG. 9: sCD146 induces U373 cell proliferation, migration and invasion in vitro and promotes CSC and EMT markers. U373 cells were treated with 100 ng/ml of VEGF, rsCD146 or combination of both molecules for 48 h and cell proliferation (A), migration (B) and invasion (C) were determined. EMT (D) and CSC (E) markers were also examined after 48 h of treatment with 100 ng/ml sCD146. Representative blots from 5 experiments are shown. Average of 3 experiments; *P<0.05, **P<0.01, ***p<0.001, experimental vs control.

FIG. 10: Effect of sCD146 and VEGF on CD146-negative glioblastoma cells. U118 cells were treated with sCD146 and/or VEFG and the cell proliferation was measured. Average of 3 experiments is shown; ** P<0.01, experimental vs control.

FIG. 11: sCD146 mediated its effects on U87 cells through a signalosome containing CD146, αvβ, and VEGFR2. Non-confluent U87 cells were used for immunoprecipitation with IgG or anti-CD146 antibody. Western-blotting was then carried out using antibodies directed against VEGFR2, CD146 and integrin subunits αv and β3. Membrane receptor EPcam was used as a negative control (A). U87 cells transfected or not with siRNA targeting β3 integrin were stimulated for 15 min with sCD146 or VEGF, and CD146 was immunoprecipitated before western blotting was done using antibody directed against total phospho-tyrosine (B). Control, CD146-KO, VEGFR2-KO or β3-KO U87 cells were stimulated for 15 min with either sCD146 or VEGF. Lysates were then subjected to western-blotting and membranes were probed with either anti-phospho β3 Y759 (C) or anti-phospho VEGFR2 Y1054 (D). Average of 3 experiments is shown; *P<0.05, **P<0.01, ***p<0.001, experimental vs control.

FIG. 12: sCD146 mediates its effects on U373 cells through a signalosome containing CD146, αvβ3, and VEGFR2. Non-confluent U373 cells were used for immunoprecipitation with IgG or anti-CD146 antibody. Western-blotting was then carried out using antibodies directed against VEGFR2, CD146 and integrin subunits αv and β3. Membrane receptor EPcam was used as a negative control (A). U373 cells transfected or not with siRNA targeting β3 integrin were stimulated for 25 min with sCD146 or VEGF, and CD146 was immunoprecipitated before western blotting was done using antibody directed against total phospho-tyrosine (B). Control, CD146-KO, VEGFR2-KO or β3-KO U87 cells were stimulated for 25 min with either sCD146 or VEGF. Lysates were then subjected to western-blotting and membranes were probed with either anti-phospho β3 Y759 (C) or anti-phospho VEGFR2 Y1054 (D). Average of 3 experiments is shown; *P<0.05, **P<0.01, ***p<0.001, experimental vs control.

FIG. 13: Integrin αvβ3 associates with membrane CD146 and VEGFR2 on HUVECs and binds sCD146. HUVECs were used for immunoprecipitation with IgG or anti-CD146 antibody. Western-blotting was then carried out using antibodies directed against VEGFR2, CD146 and integrin subunits αv and β3 (A). HUVECs transfected or not with siRNA targeting β3 integrin were stimulated for 24 h with sCD146, conditioned media from U87 cells in EBM-2 (U87-CM), or U87-CM immunodepleted from sCD146 and effect on cell proliferation (B) and migration (C) was determined. HUVECs were transfected with siRNA targeting β3 integrin and sCD146-FITC binding was determined by flow cytometry. Average of 3 experiments is shown; *P<0.05, **P<0.01, experimental vs control.

FIG. 14: CD146, integrin αvβ3, and VEGFR2 are involved for mediating sCD146 and VEGF effects on U87 cells. U87 cells or knock-out cells were stimulated with sCD146 and/or VEGF and effect on cell proliferation (A) and migration (B) was evaluated. Representative images from 3-4 experiments are shown. *P<0.05, ***p<0.001, experimental vs control.

FIG. 15: CD146, integrin αvβ3, and VEGFR2 are involved for mediating sCD146 and VEGF effects on U373 cells. U373 cells or knock-out cells were stimulated with sCD146 and/or VEGF and effect on cell proliferation (A) and migration (B) was evaluated. Representative images from 3-4 experiments are shown. *P<0.05, ***p<0.001, experimental vs control.

FIG. 16: Humanized anti-sCD146 antibody mucizumab significantly decreases U373 cell proliferation, migration and invasion and hampers CSC and EMT in vitro. U373 cells were primed with conditioned media (CM) pre-treated in the presence of irrelevant IgG, bevacizumab (Avastin), Mucizumab, or combination of both antibodies for proliferation (A), migration (B), and invasion assays (C). EMT and CSC markers were also examined at the mRNA (D and E) level. Average of 3 experiments is shown; *P<0.05, **P<0.01, ***p<0.001, experimental vs control.

FIG. 17: Humanized anti-sCD146 mucizumab significantly decreases human sCD146 and human VEGF in two different mouse models of glioblastoma. Nude mice were either subcutaneously or orthotopically injected with U87 cells and treated with IgG, bevacizumab (Avastin), mucizumab or Avastin+mucizumab. Quantification of human VEGF (A, C) and sCD146 (B, D) in the sera of treated mice was performed. 5 mice were used in each group. *P<0.05, **P<0.01, ***p<0.001, experimental vs control.

FIG. 18: Compared effects of bevacizumab, mucizumab, bevacizumab+mucizumab and bispecific anti-VEGF/sCD146 antibody on brain weight in an orthotopic model of human glioblastoma in mice. 100,000 U87 glioblastoma cells were injected orthotopically in nude mice. After 10 days, animals were iv injected twice a week for 2 weeks with 5 mg/kg of bevacizumab (Bvz), 5 mg/kg mucizumab (Mcz); 5 mg/kg bevacizumab+5 mg/kg mucizumab (Bvz+Mcz) or 5 mg/kg of the bispecific anti-VEGF/sCD146 antibody. Brain weights were determined after two weeks of treatment. Representative images are given. Results are mean values of 4 animals in each group. *P<0.05, **P<0.01, ***p<0.001 experimental vs IgG, Bvz or Mcz.

FIG. 19: Compared effects of bevacizumab, mucizumab, bevacizumab+mucizumab and bispecific anti-VEGF/sCD146 antibody on tumor size in an orthotopic model of human glioblastoma in mice. 100,000 U87 glioblastoma cells were injected orthotopically in nude mice. After 10 days, animals were iv injected twice a week for 2 weeks with 5 mg/kg of bevacizumab (Bvz), 5 mg/kg mucizumab (Mcz); 5 mg/kg bevacizumab+5 mg/kg mucizumab (Bvz+Mcz) or 5 mg/kg of the bispecific anti-VEGF/sCD146 antibody. Imaging was performed using PET-scan after two weeks of treatment by injecting 86Ga-RGD to mice and results are expressed as ratio of radioactivity between right (RH) and left (LH) hemispheres. Representative images and averages of 4-5 animals in each group are given. *: P<0.05, **: P<0.01, experimental vs IgG, Bvz or Mcz.

FIG. 20: Compared effects of bevacizumab, mucizumab, bevacizumab+mucizumab and bispecific anti-VEGF/sCD146 antibody on human CD146 mRNA expression in an orthotopic model of human glioblastoma in mice. 100,000 U87 glioblastoma cells were injected orthotopically in nude mice. After 10 days, animals were iv injected twice a week for 2 weeks with 5 mg/kg of bevacizumab (Bvz), 5 mg/kg mucizumab (Mcz); 5 mg/kg bevacizumab+5 mg/kg mucizumab (Bvz+Mcz) or 5 mg/kg of the bispecific anti-VEGF/sCD146 antibody. Human CD146 mRNA expression was determined by qPCR after two weeks of treatment. Results are mean values of 4 animals in each group. *: P<0.05, ***: P<0.001, experimental vs IgG, Bvz or Mcz.

FIG. 21: Compared effects of bevacizumab, mucizumab, bevacizumab+mucizumab and bispecific anti-VEGF/sCD146 antibody on human sCD146 and VEGF concentrations in an orthotopic model of human glioblastoma in mice. 100,000 U87 glioblastoma cells were injected orthotopically in nude mice. After 10 days, animals were iv injected twice a week for 2 weeks with 5 mg/kg of bevacizumab (Bvz), 5 mg/kg mucizumab (Mcz); 5 mg/kg bevacizumab+5 mg/kg mucizumab (Bvz+Mcz) or 5 mg/kg of the bispecific anti-VEGF/sCD146 antibody. Human sCD146 and VEGF concentrations were determined by Elisa after two weeks of treatment. Results are mean values of 4 animals in each group. *: P<0.05, experimental vs IgG, Bvz or Mcz.

FIG. 22: sCD146 concentration in responders and non-responder patients with GBM. Basal sCD146 concentration was determined by ELISA in 7 patients with GBM who responded to bevacizumab and 10 patients with GBM who did not respond to bevacizumab. *: p<0.05, non-responders vs responders by unpaired t test. Normal distribution was previously confirmed by Kolmogorov-Smirnov test for normality using SPSS software.

### DETAILED DESCRIPTION OF THE INVENTION

No curative treatment is available for cancer resistant to anti-VEGF treatment such as glioblastoma. Despite new therapeutic options, including bevacizumab, it remains a disease with poor prognosis. Bevacizumab (tradename Avastin® also identified with its CAS number as 216974-75-3) is a well-known monoclonal antibody functioning as an angiogenesis inhibitor. It works by slowing the growth of new blood vessels by inhibiting vascular endothelial growth factor A (VEGF-A). Bevacizumab treatment is often accompanied with escape mechanisms leading to pejorative evolution. Inventors herein show that, in patients with glioblastoma treated with bevacizumab, an increase in sCD146, an easily quantifiable circulating biomarker, as soon as at the end of the first cycle of treatment is associated with a decrease in progression free survival (PFS) and overall survival (OS). They have in particular shown that sCD146 constitutes an early marker predictive of resistance to this therapy when measured before the start of any cancer treatment.

CD146 (MCAM) is a transmembrane glycoprotein belonging to the immunoglobulin superfamily (Joshkon A et al., 2020). CD146 is present on the whole vascular tree, mainly at the intercellular junction of endothelial cells and regulates cell-cell cohesion, permeability and angiogenesis. Besides, soluble CD146 (sCD146), generated by ectodomain shedding of membrane CD146, induces endothelial cell proliferation and enhances angiogenesis and metastasis (Stalin et al., 2016). In cancer, the expression of (membranous) CD146 is associated with a more aggressive phenotype, malignant angiogenesis, thromboembolism, and resistance to certain chemotherapies. Alongside, sCD146 is regarded as a poor prognostic factor in various cancers (Stalin et al., 2020). Inventors deciphered the sCD146-mediated molecular mechanisms leading to bevacizumab escape. They showed in vitro and herein reveal that bevacizumab-resistant CD146+ glioblastoma cells are able to potently increase CD146 expression and enhance sCD146 secretion. The secreted sCD146 is able to exert autocrine effects on the glioblastoma cells to stimulate their proliferation, migration, and invasion. In the same way, sCD146 robustly increases cellular markers of epithelial to mesenchymal transition and cancer stem cell markers in CD146+ glioblastoma cells. To block sCD146 adverse effects, inventors generated a novel humanized monoclonal anti-sCD146 antibody, mucizumab, that specifically targets and neutralizes the soluble form of CD146 (herein identified as “sCD146”) while maintaining minimal reactivity to membrane CD146 (herein identified as “CD146”) which displays physiological functions. They further demonstrated that the combination of bevacizumab and mucizumab antibodies exhibits a therapeutic benefit by significantly decreasing Cancer Stem Cells (CSC) and Epithelial-Mesenchymal Transition (EMT) in (CD146+) bevacizumab-resistant glioblastoma cells in vitro, as well as potently decreasing tumor growth in a pre-clinical model of glioblastoma orthotopically injected in nude mice.

It is well established that epithelial to mesenchymal transition (EMT) and cancer stem cells (CSC) generation are critical for producing highly invasive and therapeutic-resistant cancer cells (Singh A. et al., 2010). Cancer cells, especially GBM, are known to secrete soluble factors that can exert autocrine effects to drive tumor progression through EMT and CSC generation (Crane et al., 2012). Here, inventors show that the expression of CD146, integrin subunits αv and β3, and VEGFR2 are upregulated in response to bevacizumab treatment in CD146+ glioblastoma cells, and that they secrete high levels of sCD146. Inventors also show that sCD146 potently induces glioblastoma cell proliferation, migration, and invasion to an extent greater than that induced by VEGF.

Inventors developed and herein describe a fully humanized monoclonal anti-sCD146 antibody, mucizumab. Mucizumab significantly reduces U87 and U373 cell proliferation, migration and invasion in vitro. Notably, combining mucizumab to bevacizumab induces a synergic inhibitory effect on cancer cell proliferation, migration and invasion when compared to bevacizumab treatment, or mucizumab treatment, as a monotherapy. Likewise, in the bevacizumab resistant cells, addition of mucizumab to the media significantly reduces the expression of EMT and CSC markers. These results were expanded and confirmed in vivo in two different U87 mouse models with ectopic and orthotopic injection of glioblastoma cells. In both cases, mucizumab was able to significantly reduce tumor growth in vivo. Importantly, higher inhibitory effect on tumor growth was observed when mucizumab and bevacizumab were combined. Of remark, bevacizumab monotherapy failed to significantly diminish tumor development in vivo, in agreement with the outcome of several phase III clinical trials (Lu K V et al., 2013). By quantifying human VEGF and human sCD146 originating from the human tumor cells in the plasma of mice, inventors observed that bevacizumab reduced hVEGF concentration but increased hsCD146. However, combining bevacizumab with mucizumab led to a potent decrease in both hVEGF and hsCD146.

Thus, inventors herein advantageously describe for the first time a humanized antibody comprising an antigen binding region that specifically binds a soluble CD146 (sCD146) protein. Said antibody comprises a light chain variable region (VL) comprising the sequence of SEQ ID NO: 1 and a heavy chain variable region (VH) comprising the sequence of SEQ ID NO: 2. A particular example of such an antibody is mucizumab.

Mucizumab is a IgG1 humanized antibody comprising an antigen binding region that specifically binds a soluble CD146 (sCD146) protein. Its VL sequence of SEQ ID NO:1 is the following: EIVLTQSPDFQSVTPKEKVTITCRASOGISTSIYWYQQKPDQSPKLLIKFASQSISGVPSRF SGSGSGTDFTLTINRVEAEDAATYYCQQSYNLPYTFGAGTKLEIK,

and its VH sequence of SEQ ID NO: 2 is the following: QVKLVESGGGVVQPGRSLTLSCAASGFTFSDYGMHWIRQAPGKGLEWIAMIYYDSSK MYYADTVKGRFTISRDNSKNTLYLQMNSLRAEDTAVYYCAAFQFDYWGQGTMVTVS S,

Wherein residues in bold character refer to CDR sequences according to IMGT, residues in underlined character refer to CDR sequence according to Kabat and wherein residues in italic character refer to backmutation.

The light chain constant region (CL) of the mucizumab of SEQ ID NO: 17 is the following:

RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC;

The heavy chain constant region (CH) of the mucizumab of SEQ ID NO: 18 is the following:

ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK.

The light chain sequence (VL+CL) of the mucizumab of SEQ ID NO: 19 is the following:

EIVLTQSPDFQSVTPKEKVTITCRASQGISTSIYWYQQKPDQSPK
LLIKFASQSISGVPSRFSGSGSGTDFTLTINRVEAEDAATYYCQQ
SYNLPYTFGAGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

The light chain amino acid sequence of SEQ ID NO: 19 is encoded by the nucleotide sequence of SEQ ID NO:21 which is the following:

GAGATTGTGCTTACTCAGTCTCCTGACTTCCAGTCCGTGACACCG
AAAGAGAAGGTCACCATCACTTGCAGAGCTTCACAAGGGATTTCC
ACCAGCATCTACTGGTATCAGCAGAAACCCGATCAATCCCCAAAG
TTGCTGATCAAGTTCGCCTCACAGAGCATAAGCGGAGTACCTTCT
CGGTTTTCTGGCAGTGGAAGTGGGACTGACTTCACACTGACCATT
AACAGGGTTGAAGCGGAAGATGCAGCCACCTATTACTGTCAGCAG
AGCTACAATCTGCCCTATACGTTTGGTGCTGGCACAAAGCTCGAG
ATCAAACGTACGGTGGCCGCTCCCAGCGTGTTCATCTTCCCCCCA
AGCGACGAGCAGCTGAAGAGCGGCACCGCCAGCGTGGTGTGTCTG
CTGAACAACTTCTACCCCAGGGAGGCCAAGGTGCAGTGGAAGGTG
GACAACGCCCTGCAGAGCGGCAACAGCCAGGAGAGCGTCACCGAG
CAGGACAGCAAGGACTCCACCTACAGCCTGAGCAGCACCCTGACC
CTGAGCAAGGCCGACTACGAGAAGCACAAGGTGTACGCCTGTGAG
GTGACCCACCAGGGCCTGTCCAGCCCCGTGACCAAGAGCTTCAAC
AGGGGCGAGTGCTGA.

The heavy chain sequence (VH+CH) of the mucizumab of SEQ ID NO:20 is the following:

QVKLVESGGGVVQPGRSLTLSCAASGFTFSDYGMHWIRQAPGKGL
EWIAMIYYDSSKMYYADTVKGRFTISRDNSKNTLYLQMNSLRAED
TAVYYCAAFQFDYWGQGTMVTVSSASTKGPSVFPLAPSSKSTSGG
TAALGCLVKDYFPEPVTVSWNSGALTSGVHTFPAVLQSSGLYSLS
SVVTVPSSSLGTQTYICNVNHKPSNTKVDKRVEPKSCDKTHTCPP
CPAPELLGGPSVFLFPPKPKDTLMISRTPEVTCVVVDVSHEDPEV
KFNWYVDGVEVHNAKTKPREEQYNSTYRVVSVLTVLHQDWLNGKE
YKCKVSNKALPAPIEKTISKAKGQPREPQVYTLPPSREEMTKNQV
SLTCLVKGFYPSDIAVEWESNGQPENNYKTTPPVLDSDGSFFLYS
KLTVDKSRWQQGNVFSCSVMHEALHNHYTQKSLSLSPGK.

The heavy chain amino acid sequence of SEQ ID NO:20 is encoded by the nucleotide amino acid sequence of SEQ ID NO: 22 which is the following:

CAGGTGAAGCTTGTCGAATCAGGTGGAGGTGTGGTTCAACCCGGA
AGGTCACTGACACTGTCTTGTGCCGCTAGTGGCTTCACCTTCAGC
GACTATGGCATGCACTGGATTCGACAGGCACCTGGGAAAGGGTTG
GAGTGGATAGCGATGATCTACTACGACTCCTCCAAGATGTACTAC
GCCGATACGGTCAAAGGGCGGTTTACCATCAGTCGCGATAACAGC
AAGAATACCCTGTATCTGCAGATGAACTCTCTCAGAGCAGAGGAT
ACTGCCGTGTACTATTGCGCTGCCTTTCAGTTCGACTATTGGGGA
CAAGGCACTATGGTGACAGTAAGCTCCGCGAGCACCAAGGGCCCA
AGCGTGTTCCCCCTGGCCCCCAGCAGCAAGAGCACCAGCGGCGGC
ACAGCCGCCCTGGGCTGCCTGGTGAAGGACTACTTCCCCGAGCCC
GTGACCGTGTCCTGGAACAGCGGAGCCCTGACCTCCGGCGTGCAC
ACCTTCCCCGCCGTGCTGCAGAGCAGCGGCCTGTACAGCCTGAGC
AGCGTGGTGACCGTGCCCAGCAGCAGCCTGGGCACCCAGACCTAC
ATCTGTAACGTGAACCACAAGCCCAGCAACACCAAGGTGGACAAG
AGAGTGGAGCCCAAGAGCTGTGACAAGACCCACACCTGCCCCCCC
TGCCCAGCCCCCGAGCTGCTGGGCGGACCCAGCGTGTTCCTGTTC
CCCCCCAAGCCCAAGGACACCCTGATGATCAGCAGAACCCCCGAG
GTGACCTGTGTGGTGGTGGACGTGTCCCACGAGGACCCAGAGGTG
AAGTTCAACTGGTACGTGGACGGCGTGGAGGTGCACAACGCCAAG
ACCAAGCCCAGAGAGGAGCAGTACAACAGCACCTACAGGGTGGTG
TCCGTGCTGACCGTGCTGCACCAGGACTGGCTGAACGGCAAGGAG
TACAAGTGTAAGGTGTCCAACAAGGCCCTGCCAGCCCCAATCGAA
AAGACCATCAGCAAGGCCAAGGGCCAGCCAAGAGAGCCCCAGGTG
TACACCCTGCCACCCAGCAGGGAGGAGATGACCAAGAACCAGGTG
TCCCTGACCTGTCTGGTGAAGGGCTTCTACCCAAGCGACATCGCC
GTGGAGTGGGAGAGCAACGGCCAGCCCGAGAACAACTACAAGACC
ACCCCCCCAGTGCTGGACAGCGACGGCAGCTTCTTCCTGTACAGC
AAGCTGACCGTGGACAAGAGCAGATGGCAGCAGGGCAACGTGTTC
AGCTGCTCCGTGATGCACGAGGCCCTGCACAACCACTACACCCAG
AAGAGCCTGAGCCTGTCCCCAGGCAAGTGA.

Humanized forms of antibodies of the invention are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin (recipient antibody) are replaced by corresponding non-human residues of the donor antibody. Furthermore, humanized antibodies may comprise residues that are not found in the recipient antibody or in the donor antibody. In general, the humanized antibody may comprise substantially all of at least one, typically two, variable domains, in which all or substantially all of the hypervariable loops correspond to those of a non-human immunoglobulin (donor antibody having the desired specificity, affinity, and capacity) and all or substantially all of the FRs are those of a human immunoglobulin sequence. Methods for humanizing non-human antibodies have been described in the art. Preferably, a humanized antibody has one or more amino acid residues introduced into it from a source, which is non-human. These non-human amino acid residues are often referred to as “import” residues, which are typically taken from an “import” variable domain. Humanization may be essentially performed by substituting hypervariable region sequences for the corresponding sequences of a human antibody. Accordingly, such humanized antibodies are chimeric wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. Other methods generally involve conferring donor CDR binding affinity onto an antibody acceptor variable region framework. One method involves simultaneously grafting and optimizing the binding affinity of a variable region binding fragment. Another method relates to optimizing the binding affinity of an antibody variable region.

The mucizumab antibody has been specifically designed by inventors via a protocol involving back-mutations. Back-mutations (as identified supra) refer to the process of antibody humanization: inventors grafted the rodent CDRs of the original antibody onto the closest human gene (they only keep the FR regions of this human gene). That is to say that the FR Framework regions or frameworks (these are the sequences between the different CDRs: FR1 before CDR1, FR2 between CDR1 and CDR2, FR3 between CDR2 and CDR3 and FR4 after CDR3) of the antibody are human. However, some of the amino acids of the FR regions are involved in the conformation of the CDRs or in the VH/VL interactions, which has a significant impact on the recognition of the antigen. Inventors identified these residues on the original sequence and on the human gene by in silico modeling and evaluated the criticality of these residues. To that end, they looked for each of the positions what were the structural/thermodynamic differences of the original residue versus the human residue. If the human residue generated a negative impact, they decided to back-mutate it with the original residue present on the rodent sequence. Finally, inventors selected 11 residues that they specifically back-mutated in order to obtain a humanized antibody having appropriate conformation and a good affinity for sCD146, which makes this humanized antibody usable for therapeutic purpose in a human being.

Therefore, in another aspect, the present invention relates to a humanized antibody comprising an antigen binding region that specifically binds a soluble CD146 (sCD146) protein wherein said antibody comprises a light chain variable region (VL) comprising the sequence of SEQ ID NO: 1 and a heavy chain variable region (VH) comprising the sequence of SEQ ID NO: 2 and wherein:
the light chain variable region (VL) sequence comprises at least one, at least two, at least three, at least four, or at least five amino acids at a position relative to the sequence set forth in SEQ ID NO:1 selected in the group consisting in 133 (isoleucine in position 33), Y34 (tyrosine in position 34), R77 (arginine in position 77), V78 (valine in position 78) and A100 (alanine in position 100), and/or
the heavy chain variable region (VH) sequence comprises at least one, at least two, at least three, at least four, at least five, or at least six amino acids at a position relative to the sequence set forth in SEQ ID NO:2 selected in the group consisting in K3 (lysine in position 3), T19 (threonine in position 19), 137 (isoleucine in position 37), 148 (isoleucine in position 48), M50 (methionine in position 50) and T63 (threonine in position 63).

In another aspect, the present description relates to a multispecific antibody, comprising an antigen binding region that specifically binds a sCD146 protein and an antigen binding region that specifically binds a VEGF protein. Preferably, the multispecific antibody of the invention is a bispecific antibody. The multispecific antibody is advantageously capable of simultaneously binding sCD146 and VEGF proteins (preferably the VEGF-A isoform).

In a preferred embodiment, the multispecific antibody is capable of inhibiting the autocrine and/or paracrine activities of VEGF and sCD146 simultaneously.

The term “multispecific antibody” as used herein refers to antibodies comprising multiple, such as two or more, e.g. three or more, different antigen-binding regions, i.e. non-naturally occurring antibodies. In particular, the multispecific antibody of the invention comprises at least one antigen-binding region that specifically binds (an ectodomain, i.e., a domain of a membrane protein that extends into the extracellular space, of) a CD146 protein, preferably of a sCD146 protein and at least one antigen-binding region that specifically binds a VEGF protein, preferably the VEGF-A isoform. This antibody may further comprise one or several other antigen-binding regions binding one or several other target antigens different from a sCD146 or of a VEGF protein provided this antibody is still capable of simultaneous binding to said two proteins and capable of inhibiting the signaling response downstream of said proteins, in particular the autocrine and/or paracrine activities of VEGF and sCD146.

Preferably, the multispecific antibody of the invention is a bispecific antibody, i.e. an antibody comprising antigen-binding regions that specifically bind two different antigens, in particular one or several antigen-binding regions that specifically bind a sCD146 protein and one or several antigen-binding regions that specifically bind a VEGF protein, preferably the VEGF-A isoform. Each antigen-binding region directed against the same antigen may be directed against the same epitope or against different epitopes of said antigen.

In the context of the present invention, the terms “antibody” and “antibodies” are used herein in the broadest sense and specifically cover full length antibody, fragments and derivatives thereof, so long as they comprise at least one antigen-binding region that specifically binds a sCD146 protein and/or at least one antigen-binding region that specifically binds a VEGF protein and exhibit the desired biological activity, i.e. inhibits the autocrine and/or paracrine activities of sCD146 and/or VEGF. It also should be understood that the term antibody, unless specified otherwise, also includes polyclonal antibodies, monoclonal antibodies (mAbs), chimeric antibodies and humanized antibodies as well as fully human antibodies.

As used herein, the term “full length antibody” refers to an antibody having a structure substantially similar to a naturally occurring immunoglobulin structure, not antibody fragments as defined below. The basic structure of a naturally occurring immunoglobulin molecule is a Y-shaped tetrameric quaternary structure consisting of two identical heavy (H) chains and two identical light (L) chains, held together by non-covalent interactions and by inter-chain disulfide bonds. In mammalian species, there are five types of heavy chains: α, δ, ε, γ, and μ, which determine the class (isotype) of immunoglobulin: IgA, IgD, IgE, IgG, and IgM, respectively. The heavy chain N-terminal variable domain (VH) is followed by a constant region, containing three domains (numbered CH1, CH2, and CH3 from the N-terminus to the C-terminus) in γ, α, and δ heavy chains, while the constant regions of p and E heavy chains are composed of four domains (numbered CH1, CH2, CH3 and CH4 from the N-terminus to the C-terminus). The CH1 and CH2 domains of IgA, IgG, and IgD are separated by a flexible hinge, which varies in length between the different classes and in the case of IgA and IgG, between the different subtypes: IgG1, IgG2, IgG3, and IgG4 have respectively hinges of 15, 12, 62 (or 77), and 12 amino acids, and IgA1 and IgA2 have respectively hinges of 20 and 7 amino acids. There are two types of light chains: λ and κ, which can associate with any of the heavy chain isotypes, but are both of the same type in a given antibody molecule. Both light chains appear to be functionally identical. Their N-terminal variable domain, termed “VL” is followed by a constant region consisting of a single domain termed “CL”. The heavy and light chains pair by protein/protein interactions between the CH1 and CL domains, and between the VH and VL domains, and the two heavy chains associate by protein/protein interactions between their CH3 domains. The effector region of immunoglobulins which is responsible for its binding to effector molecules on immune cells, corresponds to the stem of the Y-shaped structure, and contains the paired CH2 and CH3 domains of the heavy chain (or the CH2, CH3 and CH4 domains, depending on the class of antibody), and is called the Fc (for Fragment crystallizable) region. The arms of the Y-shaped structure, which consist each of the complete light chain paired with the VH and CH1 domains of the heavy chain, are called the Fab fragments (for Fragment antigen binding).

The variable regions of the heavy and light chains of the immunoglobulin molecule contain a binding domain (/region) that interacts with an antigen. The term “antigen-binding region”, as used herein, refers to a region of the antibody which interacts with an antigen and comprises both a VH and a VL regions. The VH and VL regions may be further subdivided into regions of hypervariability, also termed complementarity determining regions (CDRs), interspersed with regions that are more conserved, termed framework regions (FRs). Unless otherwise specified, the CDRs are identified according to Kabat. The FRs serves to position and align the CDRs, which form the antigen-binding site (also termed paratope). Each VH and VL is typically composed of three CDRs and four FRs, arranged from amino-terminus to carboxy-terminus in the following order: FR1, CDR1, FR2, CDR2, FR3, CDR3, FR4.

Thus, in some embodiments, the antibody of the invention is a IgG-like multispecific antibody, in particular a bispecific antibody, i.e. a bispecific antibody comprising a Fc region, and in particular a bispecific antibody comprising one Fc region and two Fab fragments, a Fab fragment that specifically binds a sCD146 protein and a Fab fragment that specifically binds a VEGF protein.

Such multi-, in particular bi-specific, immunoglobulins may be produced using any method known by the skilled person, in particular, regarding more specifically bispecific immunoglobulins, using a quadroma obtained by somatic fusion of two different hybridoma cells producing monoclonal antibodies with the desired specificity, i.e. a hybridoma cell producing a monoclonal antibody that specifically binds a sCD146 protein and a hybridoma cell producing a monoclonal antibody that specifically binds a VEGF protein, the “knob-into-hole” technology that forces heterodimerization between different heavy chains by introducing mutations in CH3 (Ridgway et al. Protein Eng, 9 (1996), pp. 617-621), and/or the CrossMab technology that provides a correct pairing of light chains by exchanging CH1 domain of one heavy chain with the constant domain of the light chain (cf. Schaefer et al. Proc Natl Acad Sci USA 2011, 108:11187-11192). The CrossMab technology is described in detail in WO 2009/080251, WO 2009/080252, WO 2009/080253, WO 2009/080254, WO 2013/026833 and WO 2016/020309. Briefly, the CrossMab technology is based on a domain crossover between heavy and light chains within one Fab-arm of a bispecific IgG, which promotes correct chain association. A CrossMab bispecific antibody of the disclosure can be a “CrossMabFab” antibody, in which the heavy and light chains of the Fab portion of one arm of a bispecific IgG antibody are exchanged. In other embodiments, a CrossMab bispecific antibody of the disclosure can be a “CrossMabVH” VL” antibody, in which the only the variable domains of the heavy and light chains of the Fab portion of one arm of a bispecific IgG antibody are exchanged. In yet other embodiments, a CrossMab bispecific antibody of the disclosure can be a “CrossMabCH” CL” antibody (also herein identified as CrossMab^{CH1/CL}), in which only the constant domains of the heavy and light chains of the Fab portion of one arm of a bispecific IgG antibody are exchanged. The multispecific antibody of the invention is preferably a CrossMab^{CH1/CL} antibody or a CrossMab^{VH-VL} antibody, even more preferably a CrossMab^{CH1/CL}.

In some embodiments, the bispecific antibodies of the disclosure are controlled Fab-arm exchange bispecific antibodies. Methods for making Fab-arm exchange bispecific antibodies are described in PCT Publication No. WO2011/131746 and Labrijn et al., 2014. Briefly, controlled Fab-arm exchange bispecific antibodies can be made by separately expressing two parental IgGI s containing single matching point mutations in the CH3 domain, mixing the parental IgGIs under redox conditions in vitro to enable recombination of half-molecules, and removing the reductant to allow reoxidation of interchain disulfide bonds, thereby forming the bispecific antibodies.

Preferably, in embodiments wherein the antibody of the invention comprises a Fc region, said Fc region is modified, preferably is an inert Fc region. The term “inert Fc region” refers to a Fc region which is at least not able to bind any Fcγ receptor, in order to prevent any interaction with the Fc receptors of different cells such as endothelial cells.

In preferred embodiments, any antibody of the invention is an immunoglobulin fragment or is formed from immunoglobulin fragments. In particular, in these embodiments, the antibody preferably does comprise a Fc region.

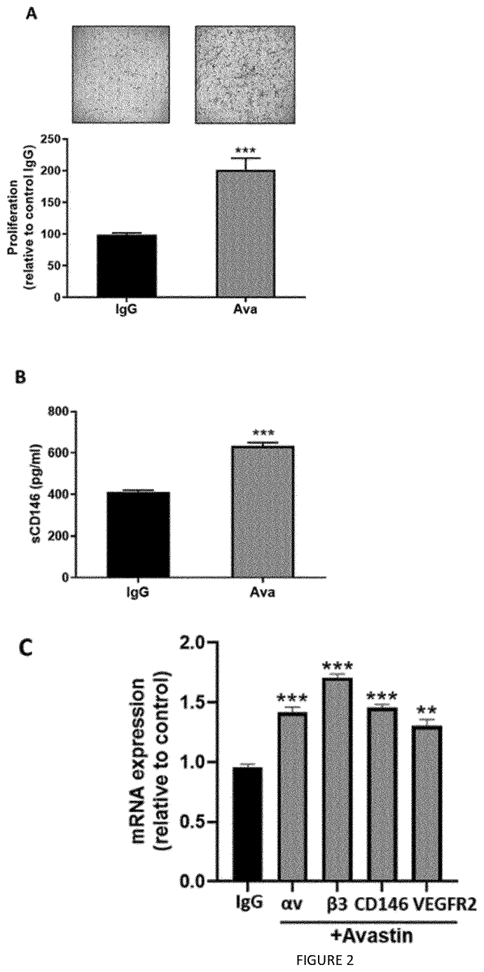

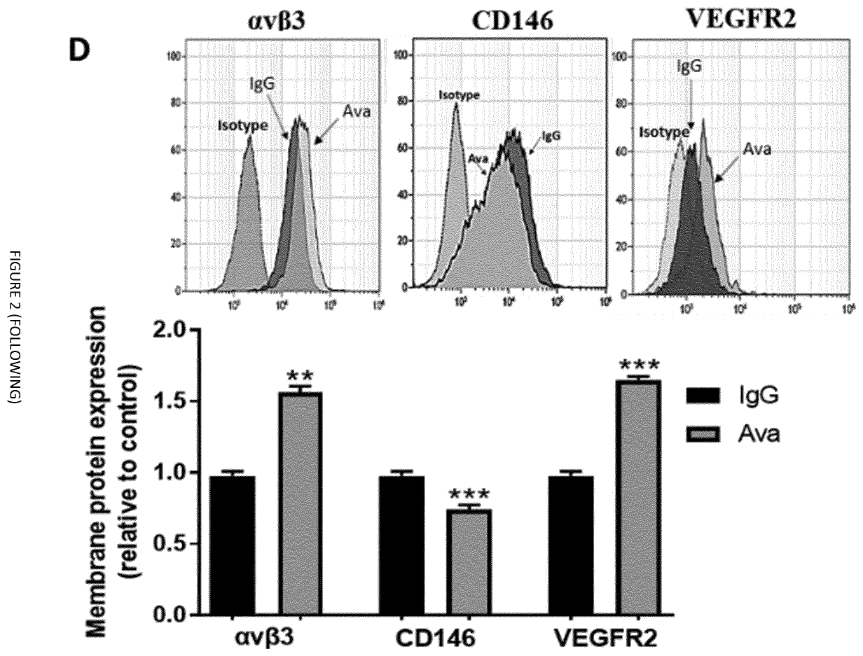

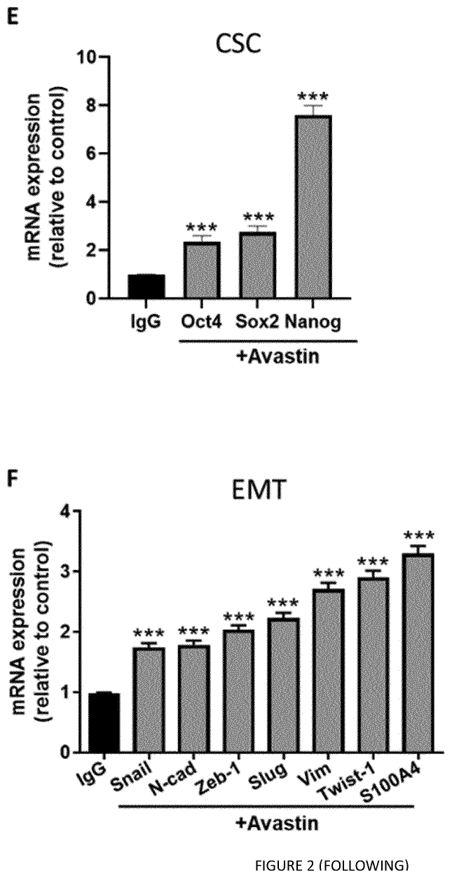

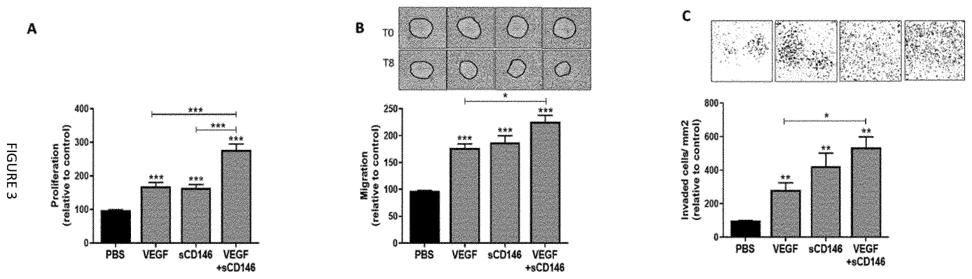

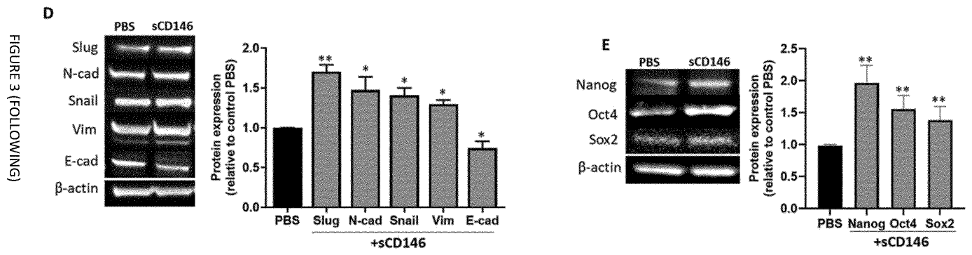

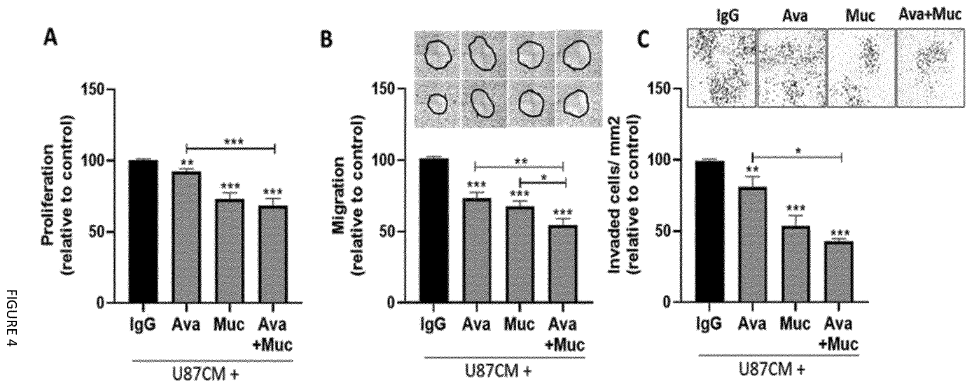

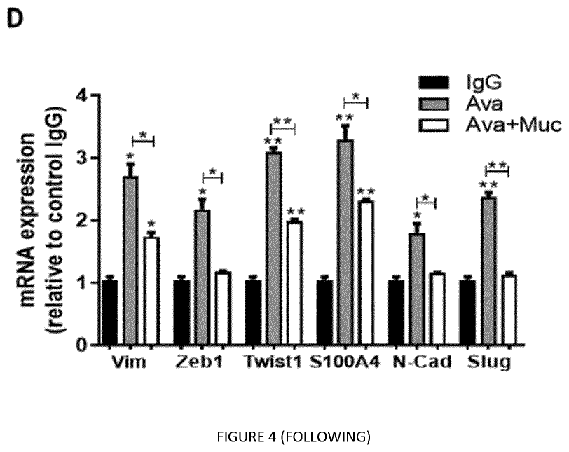

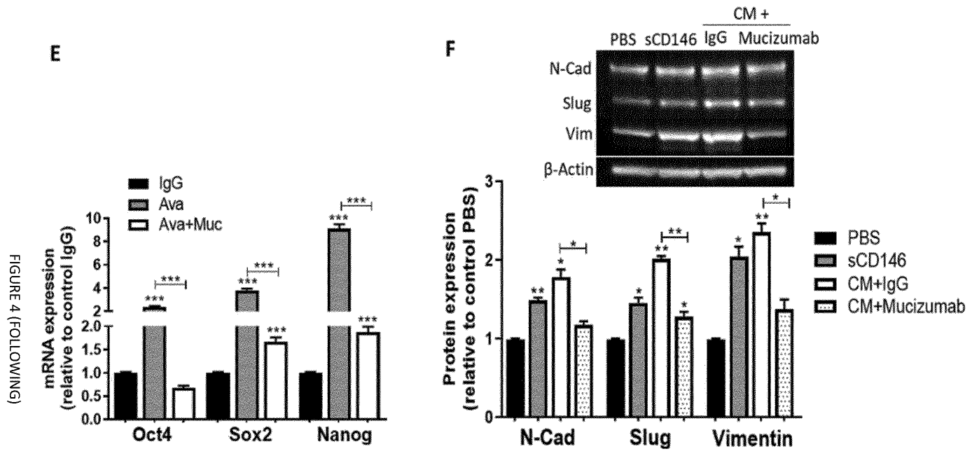

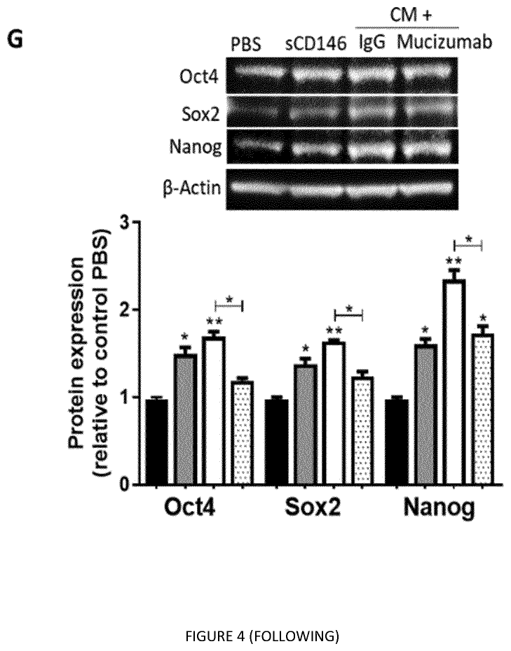

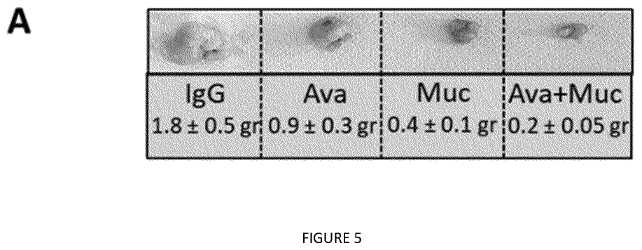

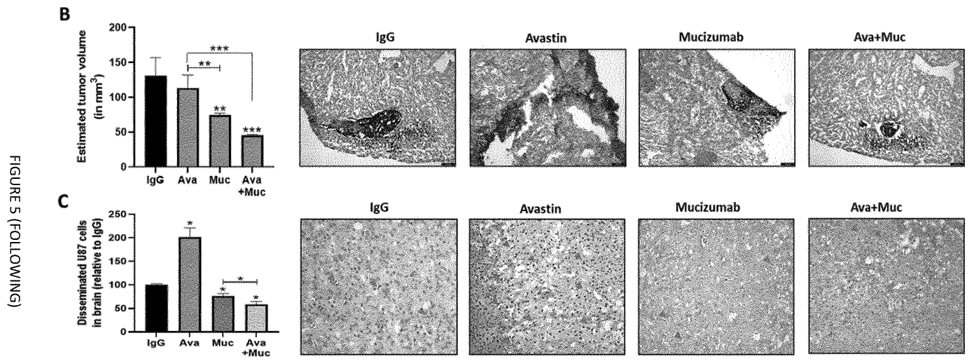

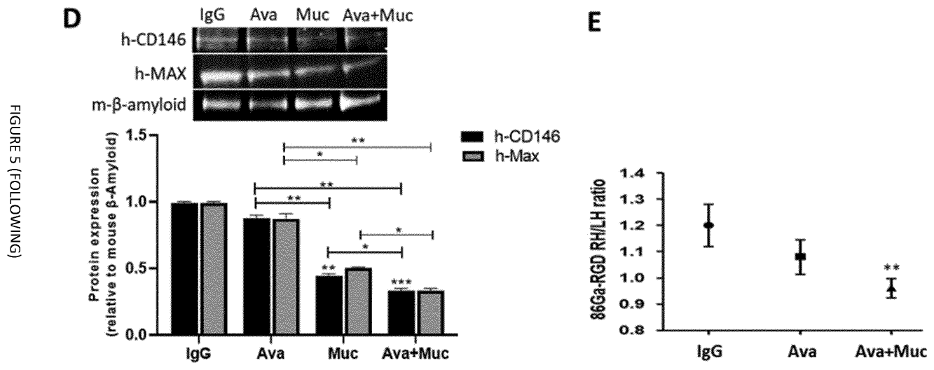

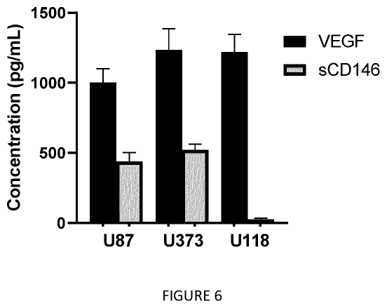

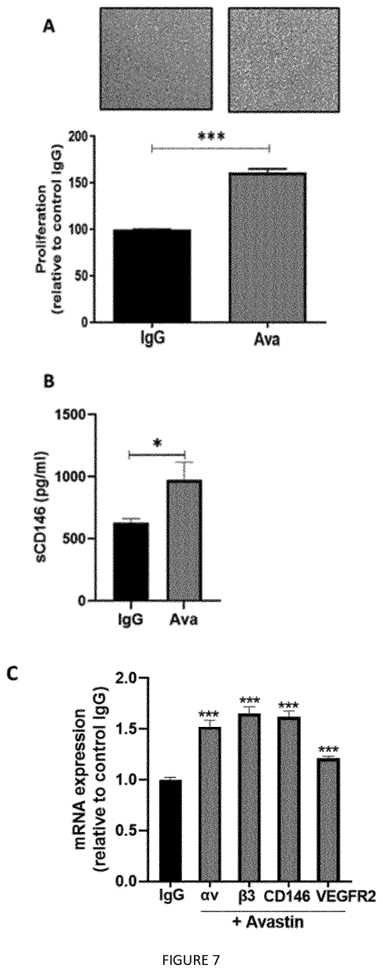

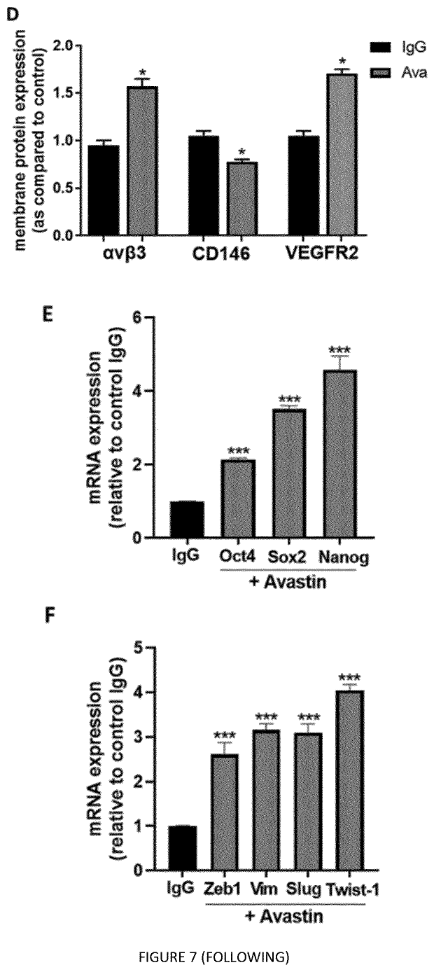

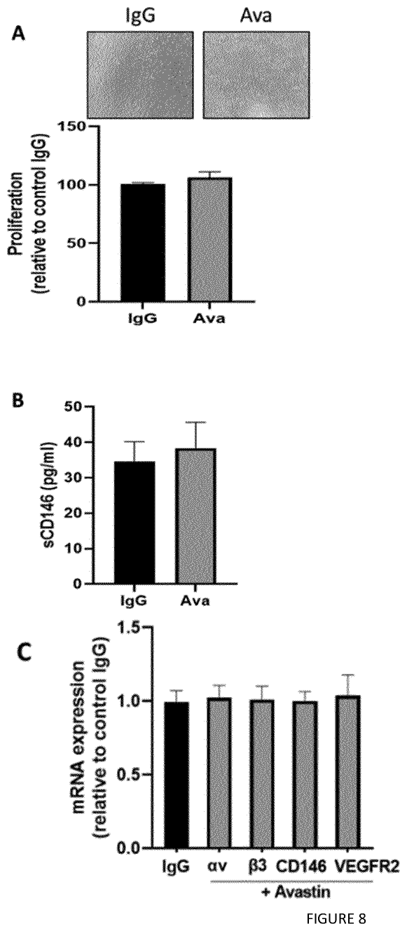

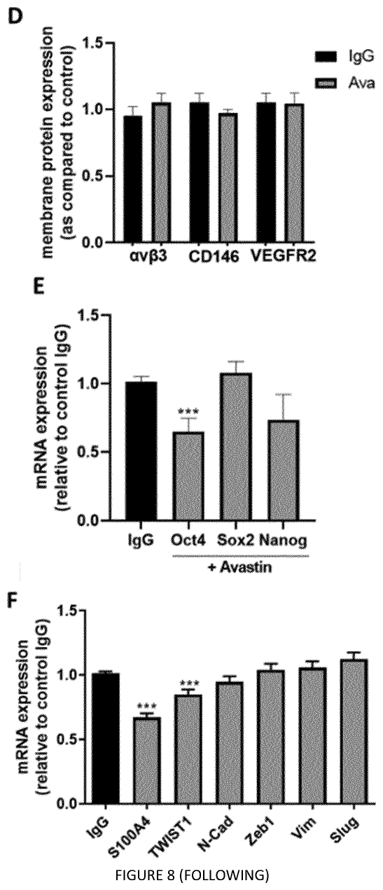

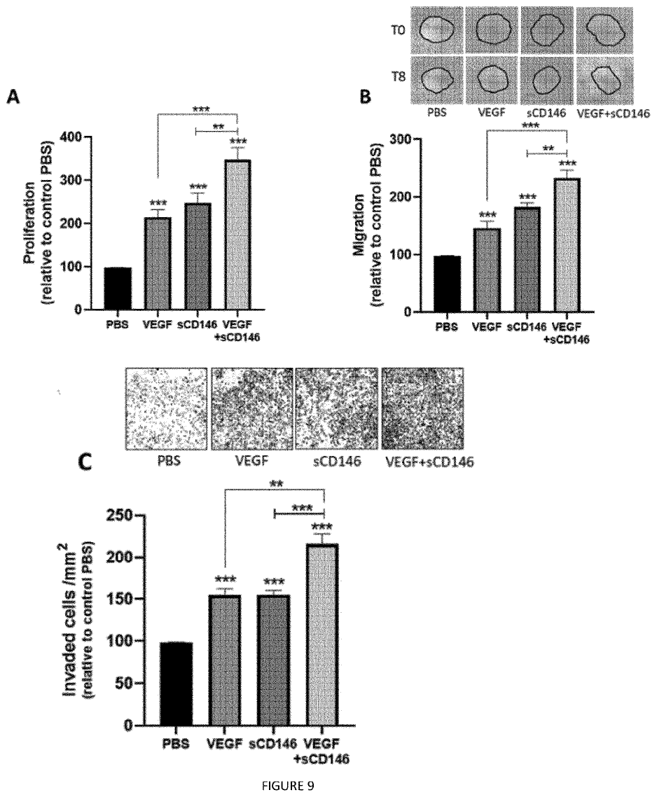

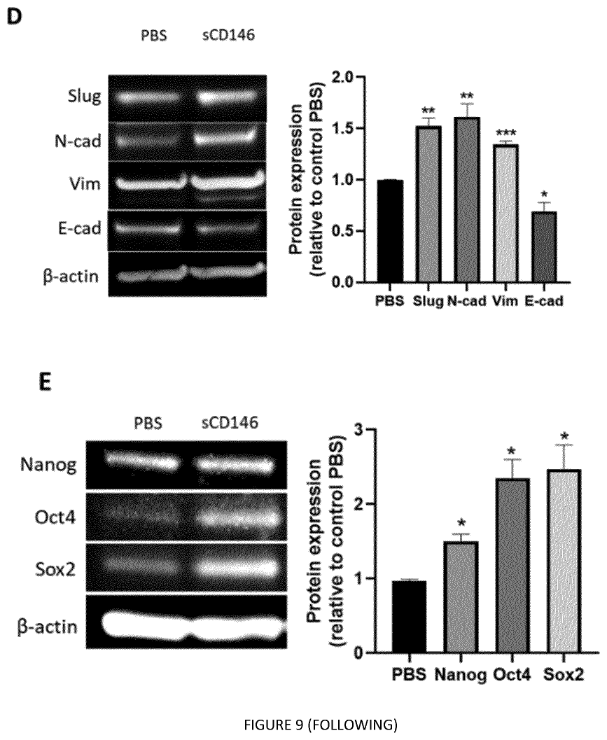

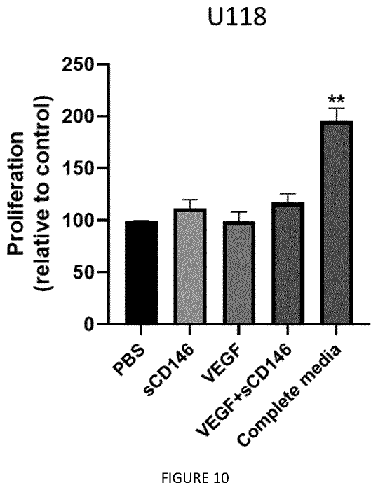

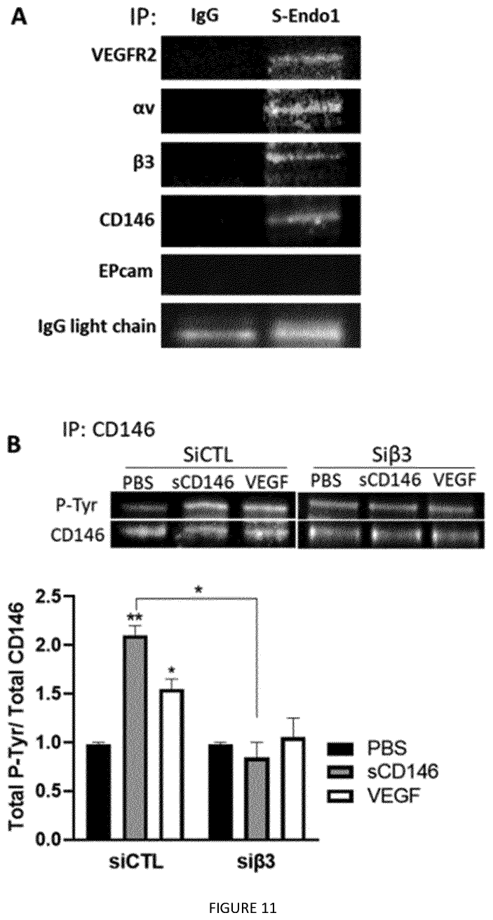

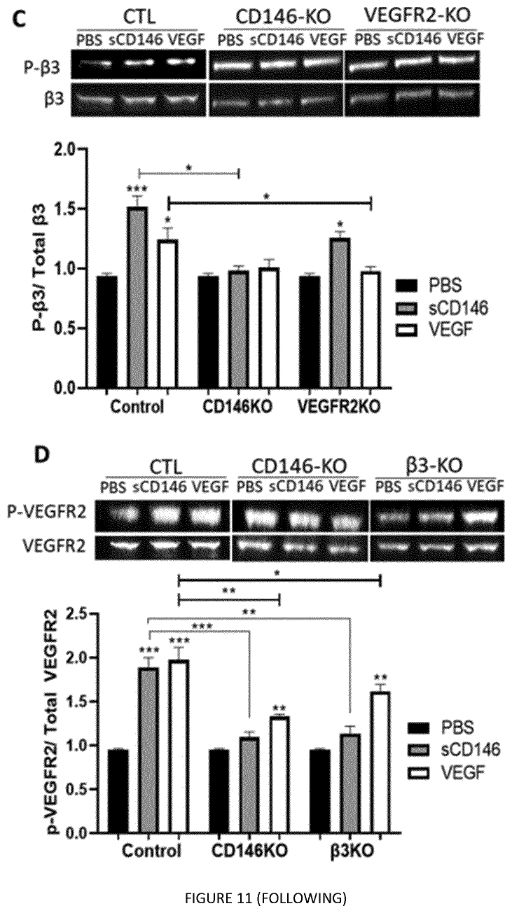

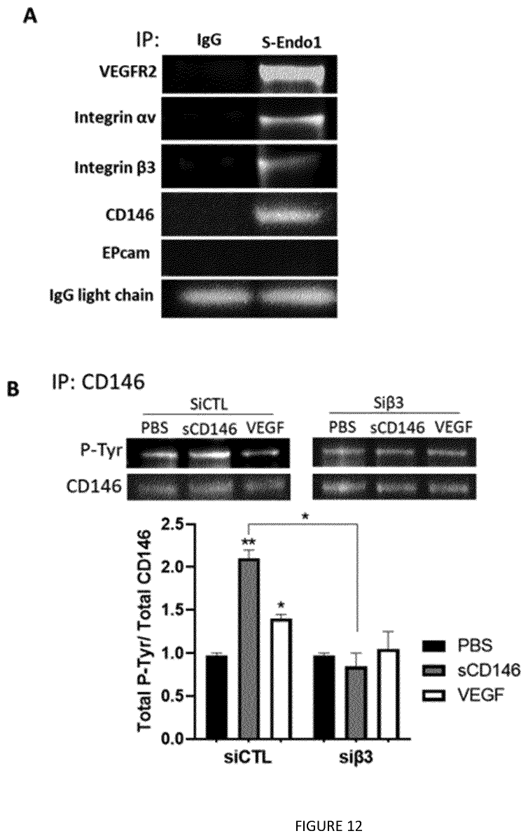

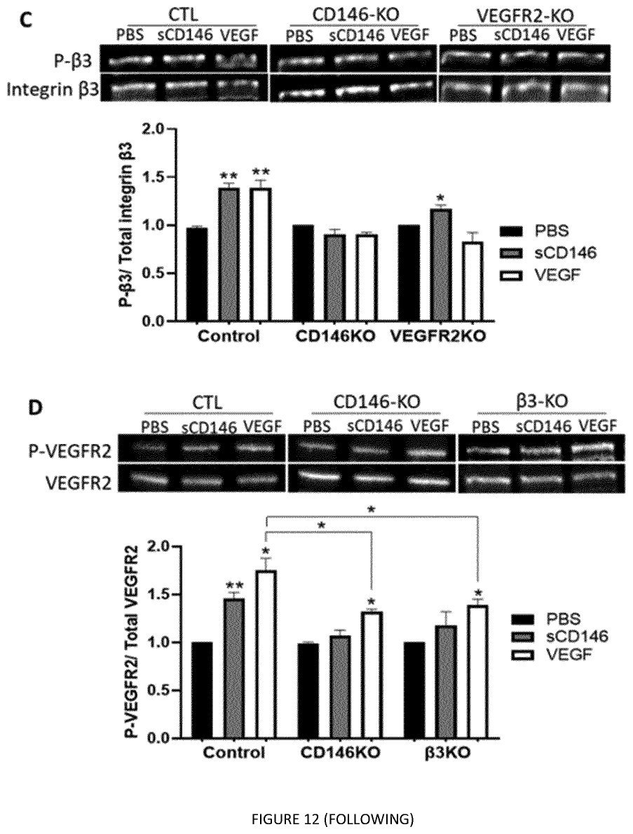

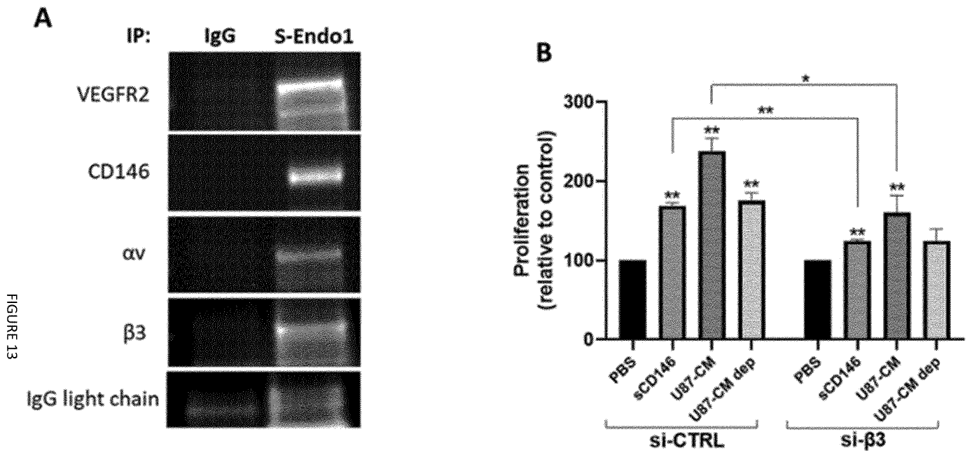

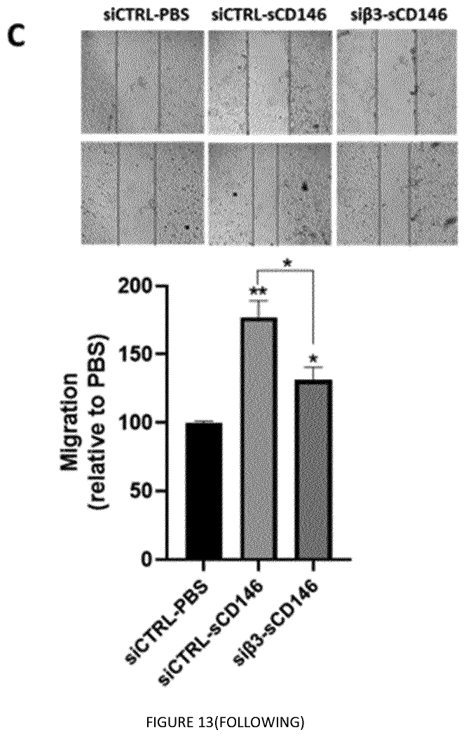

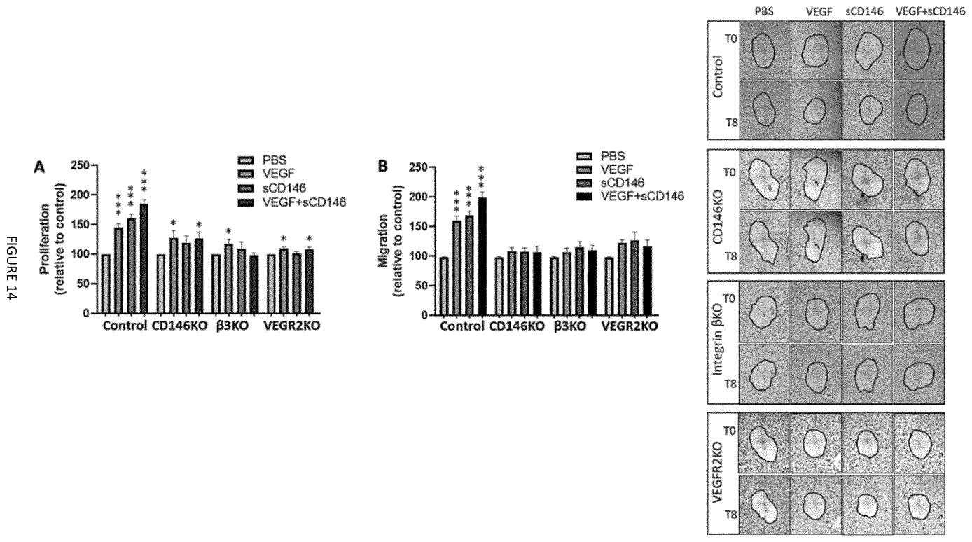

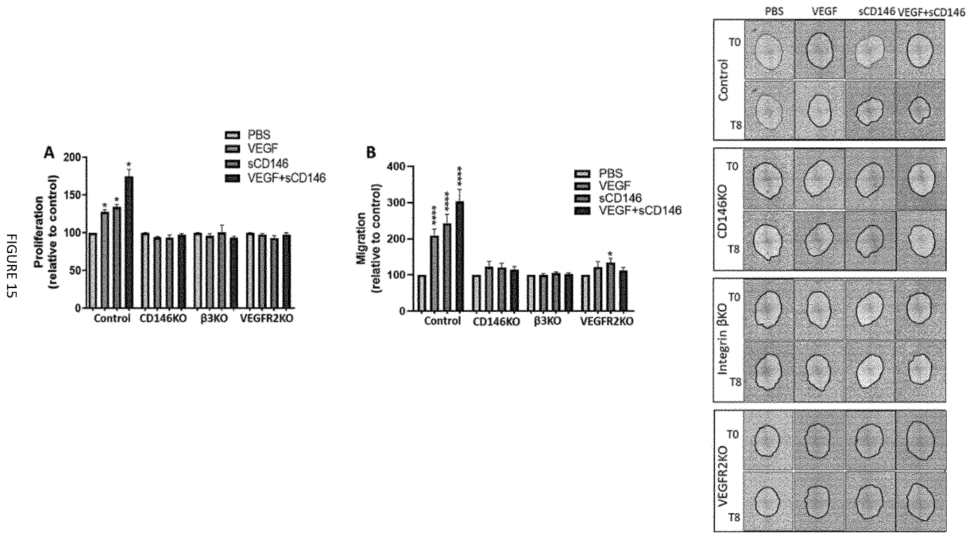

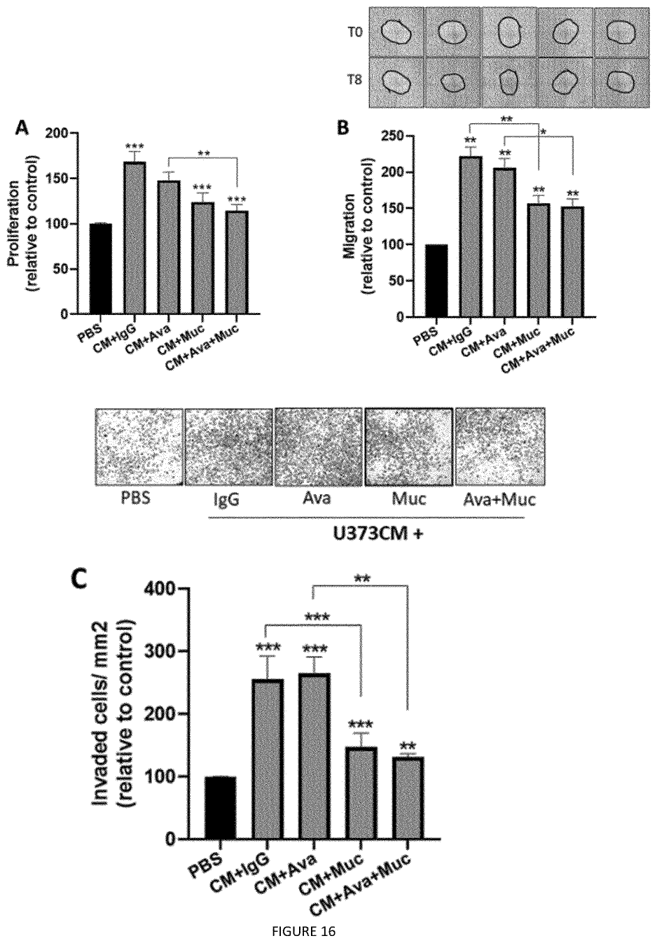

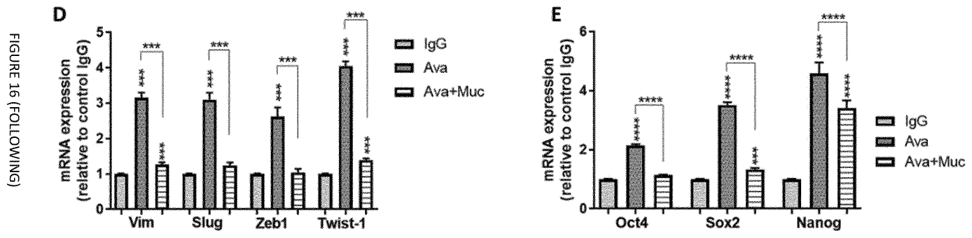

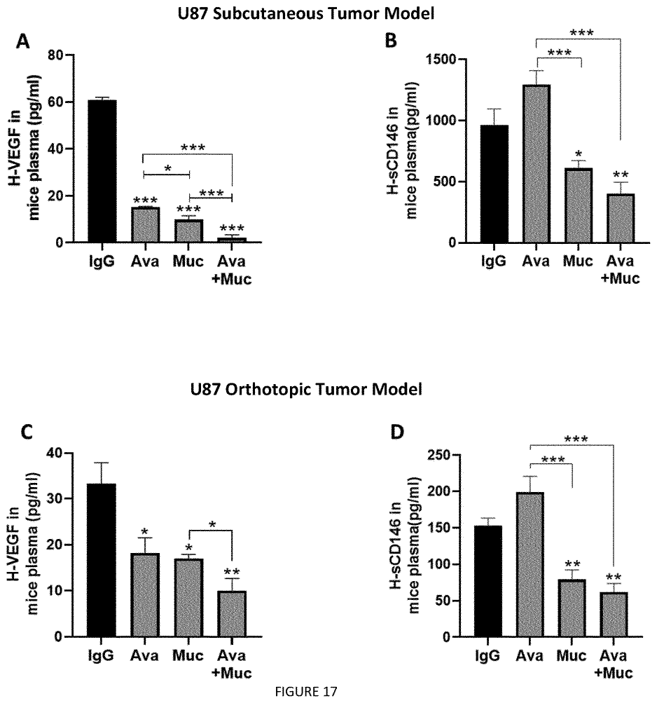

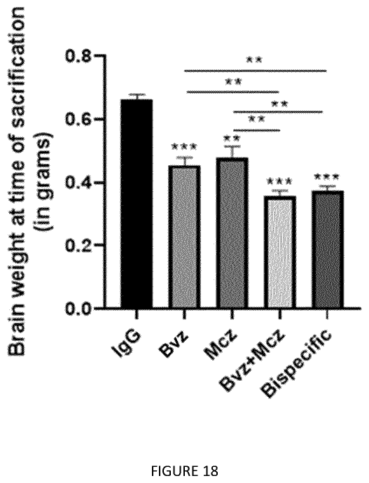

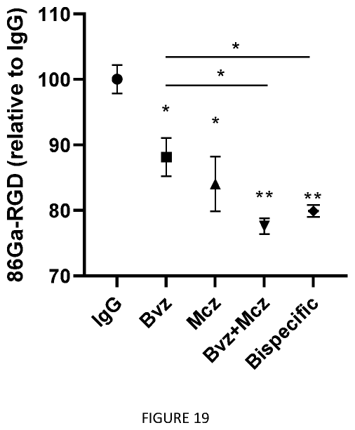

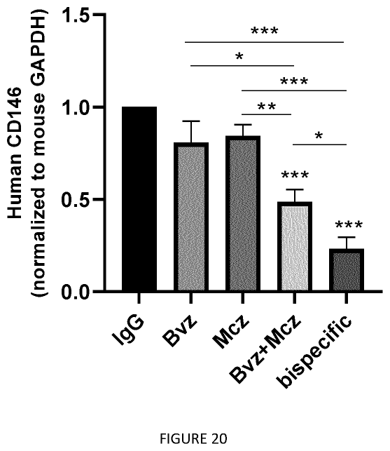

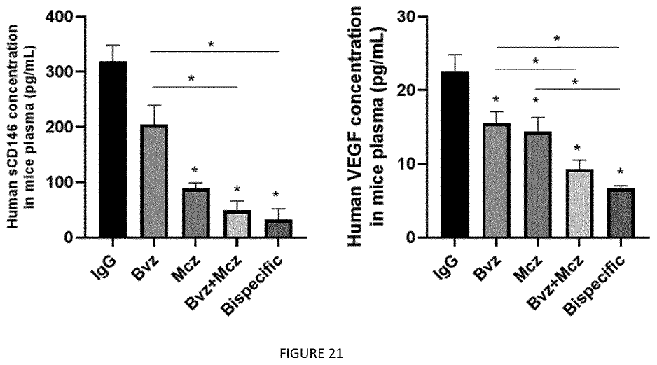

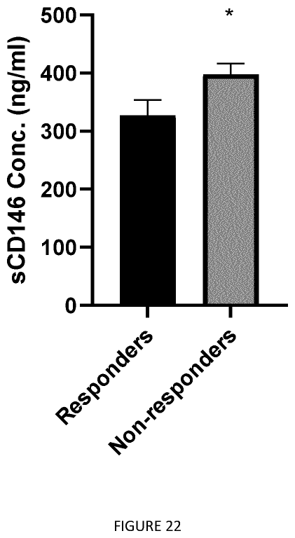

In particular, any antibody of the invention may be of any suitable Fc-less bispecific format, including but being not limited to, bispecific F(ab′)₂ format obtained by chemical cross-linking of two different Fab fragments, tandem Fab, tandem scFv (BiTE®) format obtained by tandem joining of two different scFv fragments, diabody format obtained by heterodimer formation of two different scFvs, tandem diabody format obtained by linking two diabodies, single chain diabodies (scDbs), bispecific nanobodies, heterodimeric Fab format obtained by association of two different Fab fragments fused with a leucine zipper domain, minibody format obtained by fusing a scFv to the N-terminus of the CH3 domain and another scFv to the other CH3 domain, with the CH3 domains further stabilized by C-terminal disulfide bonds, and various other formats such as disclosed in the article of Brinkmann and Kontermann, MAbs. 2017 February-March; 9(2): 182-212, incorporated herein by reference, in particular in FIG. 2.

The format of the antibody of the invention can be easily chosen by the skilled person depending on the therapeutic indication of interest. Indeed, various formats and strategies are available to generate recombinant bispecific antibodies and well known for the skilled person (see e.g. Brinkmann and Kontermann, MAbs. 2017 February-March; 9(2): 182-212). The format has to be chosen in order to allow simultaneous binding to a sCD146 protein and to a VEGF protein. This capacity can be easily assessed by any method known by the skilled person such as ELISA or Dot-Blot.

In some particular aspects, the antibody of the invention is of a format selected from the group consisting of tandem scFv, scDbs, tandem Fab, F(ab′)₂ and minibodies, preferably is a bispecific F(ab′)₂ format obtained by chemical cross-linking of two different Fab fragments

In a particular aspect, the antibody of the invention is obtained via the CrossMab technology and the knobs-into-holes technology, as disclosed in the experimental section.

In a particular embodiment, the antibody of the invention is a monoclonal antibody.

A “monoclonal antibody”, as used herein, designates an antibody arising from a nearly homogeneous population of antibodies. More particularly, the antibodies of a given subject are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one isotype and subtype, and light chains of only one type. In addition, in contrast with preparations of polyclonal antibodies, each monoclonal antibody is directed to a single epitope of an antigen.

The term “antibody derivative”, as used herein, refers to an antibody provided herein, wherein one or more of the amino acids are chemically modified, e.g. by alkylation, PEGylation, acylation, ester or amide formation or the like. In particular, this term may refer to an antibody provided herein that is further modified to contain additional non proteinaceous moieties that are known in the art and readily available. The moieties suitable for derivatization of the antibody include but are not limited to water soluble polymers. Examples of water soluble polymers include, but are not limited to, PEG, copolymers of ethylene glycol/propylene glycol, carboxymethylcellulose, dextran and polyvinyl alcohol. The derivative may also be an immunoconjugate comprising an antibody of the invention conjugated to one or more heterologous molecule(s), including but not limited to a detectable moiety such as a fluorescent moiety; or to a solid support, such as agarose beads or the like. The linkers between the antibody and the heterologous molecule(s) may be a “cleavable linker” such as an acid-labile linker, peptidase-sensitive linker, photolabile linker, dimethyl linker or disulfide-containing linker (Chari et al., Cancer Res. 52: 127-131 (1992)).

In some embodiments, the antibody of the invention may be part of a larger fusion molecule, formed by covalent or non-covalent association of the antibody with one or more other molecules such as proteins.

A particular antibody of the invention comprises at least one antigen binding region that specifically binds a sCD146 protein and at least one antigen binding region that specifically binds a VEGF protein, preferably the VEGF-A isoform.

The multispecific antibodies of the invention are capable of simultaneous binding to a sCD146 and a VEGF protein. Due to this simultaneous binding, the antibody is capable of inhibiting the signaling response downstream of the VEGF and thus of preventing angiogenic activity of VEGF, and is capable of inhibiting the signaling response downstream of sCD146 and thus of preventing paracrine and/or autocrine effects of sCD146. The activity of this antibody can be assessed by any method known by the skilled person such as proliferation assay.

An “antigen binding region” “specifically binds” a target antigen when it has a significantly higher binding affinity for, and consequently is capable of distinguishing, that antigen compared to its affinity for other unrelated proteins, under similar binding assay conditions. “Specific binding” does not necessarily require (although it can include) exclusive binding. Generally, but not necessarily, reference to binding means preferential binding. Preferably, the antigen binding region will not show any significant binding to ligands other than its specific targets (e.g., an affinity of about 100-fold less), i.e. minimal cross-reactivity. Antibody specificity may be determined by measurement of cross-reactivity using well-known methods such as ELISA binding assays. Binding may be considered specific when the binding affinity is 10⁻⁶ M (K_{D}) or more. In particular, binding may be considered specific when binding affinity is about 10⁻⁸ to 10⁻¹¹ M (K_{D}), or of about 10⁻⁹ to 10⁻¹¹ M or even higher.

A particular antibody of the invention comprises at least one antigen binding region that specifically binds a sCD146 protein, such as mucizumab.

As used herein, the term “sCD146” refers to a soluble CD146 protein, preferably to a human sCD146 protein having an amino acid sequence selected from SEQ ID NO: 57, SEQ ID NO: 58, SEQ ID NO: 59, SEQ ID NO: 60, SEQ ID NO: 61, SEQ ID NO: 62 and SEQ ID NO: 63, respectively encoded by SEQ ID NO: 50, SEQ ID NO: 51, SEQ ID NO: 52, SEQ ID NO: 53, SEQ ID NO: 54, SEQ ID NO: 55 and SEQ ID NO: 56. The polynucleotide and amino acid sequences are well-known in the art.

The antibody may be a competitive inhibitor of the binding of a sCD146 receptor such as angiomotin or integrin backmutation αvβ3, i.e. an antibody that binds to a sCD146 and that significantly reduces or inhibits the binding of a sCD146 receptor to the ectodomain of said protein. Competition assays can be performed using standard techniques in the art (for instance, competitive ELISA or other binding assays). In this case, the antibody of the invention may reduce or inhibit the binding of sCD146 to angiomotin and/or integrin αvβ3 by at least 10%, at least 25%, at least 50%, at least 75%, or at least 90% (percent of ligand blocked at saturating levels of antibodies based on competitive ELISA).

The antibody of the invention may comprise, in addition to the at least one antigen binding region that specifically binds a sCD146 protein, at least one antigen binding region that specifically binds a VEGF protein, preferably the VEGF-A isoform.

As used herein, the term “VEGF” refers to Vascular Endothelial Growth Factor, a signal protein produced by many cells that stimulates the formation of blood vessels. In mammals, the VEGF family comprises five members: VEGF-A, placenta growth factor (PGF), VEGF-B, VEGF-C and VEGF-D. As used herein, VEGF preferably refers to a human VEGF protein, even more preferably to a VEGF-A isoform such as the isoform having an amino acid sequence of SEQ ID NO: 43: MNFLLSWVHWSLALLLYLHHAKWSQAAPMAEGGGQNHHEVVKFMDVYQRSYCHPI ETLVDIFQEYPDEIEYIFKPSCVPLMRCGGCCNDEGLECVPTEESNITMQIMRIKPHQGQ HIGEMSFLQHNKCECRPKKDRARQEKKSVRGKGKGQKRKRKKSRYKSWSVYVGARC CLMPWSLPGPHPCGPCSERRKHLFVQDPQTCKCSCKNTDSRCKARQLELNERTCRCDK PRR. The polynucleotide and amino acid sequences of VEGF-A are well-known in the art and can be identified for example with their accession number NP_003367.4, NP_001020537.2, NP_001020538.2, NP_001020539.2, NP 001020540.2, NP 001020541.2, NP_001028928.1, NP_001165093.1, NP_001165094.1, NP_001165095.1, NP 001165096.1, NP_001165097.1, NP_001165098.1, NP_001165099.1, NP 001165100.1, NP 001165101.1, NP_001191313.1, NP_001191314.1, NP_001273973.1, NP_001303939.1

The antibody may be a competitive inhibitor of the binding of a VEGF receptor such as VEGFR2, i.e. an antibody that binds to a VEGF and that significantly reduces or inhibits the binding of a VEGF to said VEGF receptor. Again, competition assays can be performed using standard techniques in the art (for instance, competitive ELISA or other binding assays). In this case, the antibody of the invention may reduce or inhibit the binding of a VEGF to a VEGF receptor by at least 10%, at least 25%, at least 50%, at least 75%, or at least 90% (percent of ligand blocked at saturating levels of antibodies based on competitive ELISA). Alternatively, the antibody of the invention may not interfere with ligand binding.

The position in a polypeptide corresponding to a specific residue of SEQ ID NO: 43 or any other specific sequence, may be easily determined by the skilled person, for example using a global alignment algorithm such as Needleman and Wunsch algorithm. More particularly, the antibody of the invention may comprise an antigen binding region that specifically binds a VEGF epitope defined by at least one, two, three or more of residues of SEQ ID NO: 43.

A particular multispecific antibody comprises an antigen binding region that specifically binds a sCD146 protein and which comprises a light chain variable region (L) comprising one or more of the following CDRs:
H-CDR1 of SEQ ID NO: 3 or a variant thereof,
H-CDR2 of SEQ ID NO: 4 or a variant thereof, and
H-CDR3 of SEQ ID NO: 5 or a variant thereof,
   and/or a heavy chain variable region (H) comprising one or more of the following CDRs:
L-CDR1 of SEQ ID NO: 6 or a variant thereof,
L-CDR2 of SEQ ID NO: 7 or a variant thereof, and
L-CDR3 of SEQ ID NO: 8 or a variant thereof
   said variants having at least 80% sequence identity, i.e., as explained in the below paragraph, at least 85%, at least 90%, at least 95%, at least 96%, at least 97%, at least 98% or at least 99%, to the recited CDR sequences, preferably having one, two or three amino acid variations from the recited CDR sequences.

As used herein, the term “variant” generally refers to a sequence having at least 80%, at least 85%, at least 90%, at least 95%, at least 96%, at least 97%, at least 98% or at least 99% sequence identity to the recited sequence (whatever the specific nature of the antibody sequence selected among those herein described by inventors), provided that the antigen binding region retains the ability to bind to the target, e.g. a sCD146 or VEGF protein, typically the VEGF-A isoform. Preferably, this term refers to a sequence having one, two or three amino acid variations from the recited sequence, in particular from the recited CDR sequence. In particular, the amino acid variations in the sequences may be conservative amino acid substitutions.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://www.ncbi.nlm.nih.gov/igblast/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5. “Conservative” amino acid substitutions are those in which an amino acid residue is replaced with an amino acid residue having a side chain with similar physicochemical properties. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

In a preferred aspect, said multipecific antibody comprises a light chain variable region (VL) comprising the sequence of SEQ ID NO: 1 or a variant thereof, and a heavy chain variable region (VH) comprising the sequence of SEQ ID NO: 2 or a variant thereof, said variants having at least 80% sequence identity to the recited sequences, i.e., as explained in the below paragraph, at least 85%, at least 90%, at least 95%, at least 96%, at least 97%, at least 98% or at least 99%, preferably having one, two or three amino acid variations from the recited sequences.

In a more particular embodiment, the antibody of the invention comprises an antigen binding region that specifically binds a sCD146 protein, wherein said sCD146 protein comprises the following sequence:

(SEQ ID NO: 57)
MGLPRLVCAFLLAACCCCPRVAGVPGEAEQPAPELVEVEVGSTAL
LKCGLSQSQGNLSHVDWFSVHKEKRTLIFRVRQGQGQSEPGEYEQ
RLSLQDRGATLALTQVTPQDERIFLCQGKRPRSQEYRIQLRVYKA
PEEPNIQVNPLGIPVNSKEPEEVATCVGRNGYPIPQVIWYKNGRP
LKEEKNRVHIQSSQTVESSGLYTLQSILKAQLVKEDKDAQFYCEL
NYRLPSGNHMKESREVTVPVFYPTEKVWLEVEPVGMLKEGDRVEI
RCLADGNPPPHFSISKQNPSTREAEEETTNDNGVLVLEPARKEHS
GRYECQAWNLDTMISLLSEPQELLVNYVSDVRVSPAAPERQEGSS
LTLTCEAESSQDLEFQWLREETDQVLERGPVLQLHDLKREAGGGY
RCVASVPSIPGLNRTQLVKLAIFGPPWMAFKERKVWVKENMVLNL
SCEASGHPRPTISWNVNGTASEQDQDPQRVLSTLNVLVTPELLET
GVECTASNDLGKNTSILFLELVNLTTLTPDSNTTTGLSTSTASPH
TRANSTSTERKL.

Alternatively, the sequence of the antigen binding region that specifically binds a sCD146 protein may comprise the heavy variable chain, the light variable chain or the CDR sequences of any known antibody targeting specifically a sCD146 protein.

In a particular aspect of the invention, the antibody directed against a sCD146 protein is mucizumab.

The antibody of the invention may also comprise at least one antigen binding region that specifically binds a VEGF protein, preferably a VEGF-A isoform.

Another particular antibody, which may be a multispecific antibody, of the invention comprises an antigen binding region that specifically binds a VEGF protein and which comprises a light chain variable region (L) comprising one or more of the following CDRs:
H-CDR1 of SEQ ID NO: 23 or a variant thereof,
H-CDR2 of SEQ ID NO: 24 or a variant thereof, and
H-CDR3 of SEQ ID NO: 25 or a variant thereof,
   and/or a heavy chain variable region (H) comprising one or more of the following CDRs:
L-CDR1 of SEQ ID NO: 26 or a variant thereof,
L-CDR2 of SEQ ID NO: 27 or a variant thereof, and
L-CDR3 of SEQ ID NO: 28 or a variant thereof,
   said variants having at least 80% sequence identity to the recited CDR sequences, i.e., as explained in the below paragraph, at least 85%, at least 90%, at least 95%, at least 96%, at least 97%, at least 98% or at least 99%, preferably having one, two or three amino acid variations from the recited CDR sequences.

In a preferred aspect, said multipecific antibody, comprises a light chain variable region (VL) comprising the sequence of SEQ ID NO: 37 or a variant thereof, and a heavy chain variable region (VH) comprising the sequence of SEQ ID NO: 38, or a variant thereof, said variants having at least 80% sequence identity to the recited sequences, i.e., as explained in the below paragraph, at least 85%, at least 90%, at least 95%, at least 96%, at least 97%, at least 98% or at least 99%, preferably having one, two or three amino acid variations from the recited sequences.

In a more particular embodiment, the antibody of the invention comprises an antigen binding region that specifically binds a VEGF-A isoform protein and which comprises for example the following sequence:

(SEQ ID NO: 43)
MNFLLSWVHWSLALLLYLHHAKWSQAAPMAEGGGQNHHEVVKFMD
VYQRSYCHPIETLVDIFQEYPDEIEYIFKPSCVPLMRCGGCCNDE
GLECVPTEESNITMQIMRIKPHQGQHIGEMSFLQHNKCECRPKKD
RARQEKKSVRGKGKGQKRKRKKSRYKSWSVYVGARCCLMPWSLPG
PHPCGPCSERRKHLFVQDPQTCKCSCKNTDSRCKARQLELNERTC
RCDKPRR

Alternatively, the sequence of the antigen binding region that specifically binds a VEGF protein may comprise the heavy variable chain, the light variable chain or the CDR sequences of any known antibody targeting a VEGF protein such as for example ranibizumab, in particular a commercially available anti-VEGF antibody such as bevacizumab (Avastin®) or ranibizumab (Lucentis®, CAS no: 347396-82-1).

In a particular aspect of the invention, the antibody directed against VEGF is bevacizumab.

In another particular aspect of the invention, the antibody directed against the VEGF-A isoform is bevacizumab.

Also herein described are multispecific antibodies comprising several or each of the herein above described CDR sequences and/or variables sequences, i.e., several or each of SEQ ID NO: 1-8, 23-28, 37 and 38.

A preferred multispecific antibody, in particular bispecific antibody, according to the invention comprises an antigen binding region that specifically binds a sCD146 protein and an antigen binding region that specifically binds a VEGF protein, preferably the VEGF-A isoform, and which comprises a first light (L) and a first heavy (H) chain variable region directed against a sCD146 protein, and a second light (L) and a second heavy (H) chain variable region directed against a VEGF protein, wherein the first light (L) chain variable regions directed against a sCD146 protein comprise one or more of the following CDRs:
H-CDR1 of SEQ ID NO: 3 or a variant thereof,
H-CDR2 of SEQ ID NO: 4 or a variant thereof, and
H-CDR3 of SEQ ID NO: 5 or a variant thereof,
   and/or the first heavy (H) chain variable region comprising one or more of the following CDRs:
L-CDR1 of SEQ ID NO: 6 or a variant thereof,
L-CDR2 of SEQ ID NO: 7 or a variant thereof, and
L-CDR3 of SEQ ID NO: 8 or a variant thereof,
   and wherein the second light (L) chain variable region directed against a VEGF protein comprise one or more of the following CDRs:
H-CDR1 of SEQ ID NO: 23 or a variant thereof,
H-CDR2 of SEQ ID NO: 24 or a variant thereof, and
H-CDR3 of SEQ ID NO: 25 or a variant thereof,
   and/or the second heavy (H) chain variable region comprising one or more of the following CDRs:
L-CDR1 of SEQ ID NO: 26 or a variant thereof,
L-CDR2 of SEQ ID NO: 27 or a variant thereof, and
L-CDR3 of SEQ ID NO: 28 or a variant thereof,
   said variants having at least 80%, preferably at least 85%, at least 90%, at least 95%, at least 96%, at least 97%, at least 98% or at least 99% sequence identity to the recited CDR sequences, preferably having one, two or three amino acid variations from the recited CDR sequences.

In a preferred aspect, said multipecific antibody, comprises:
a first light chain variable region comprising, or consisting of, the sequence of SEQ ID NO: 1 or a variant thereof,
a first heavy chain variable region comprising, or consisting of, the sequence of SEQ ID NO: 2 or a variant thereof,
a second light chain variable region comprising, or consisting of, the sequence of SEQ ID NO: 37 or a variant thereof, and
a second heavy chain variable region comprising or consisting of, the sequence of SEQ ID NO: 38 or a variant thereof, said variants having at least 80%, preferably at least 85%, at least 90%, at least 95%, at least 96%, at least 97%, at least 98% or at least 99% sequence identity to the recited sequences.

In another preferred aspect, said multispecific antibody comprises a a light chain constant region (CL) of sequence: TVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO: 39) and/or of sequence SEQ ID NO: 17, or variant(s) thereof and/or a heavy chain constant region (CH) of sequence: ASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKVEPKSCDKTHTCPPCPAPELLG GPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 40) and/or of sequence SEQ ID NO: 18, or variant(s) thereof, said variants having at least 80%, preferably at least 85%, at least 90%, at least 95%, at least 96%, at least 97%, at least 98% or at least 99% sequence identity to the recited sequences.

The CDRs as described above may be associated with any framework region(s).

By “framework” or “FR” region as used herein is meant a region of an antibody variable domain exclusive of those regions defined as CDRs. Each antibody variable domain framework can be further subdivided into the contiguous regions separated by the CDRs (FR1, FR2, FR3 and FR4). Preferably, the framework region is of human origin. In particular, the antibodies of the invention may comprise any suitable framework variable domain sequence(s), provided the binding activity to a sCD146 and/or VEGF protein(s) is retained.

In particular embodiments, the variable domain framework(s) of the antibody binding region that specifically binds a sCD146 protein may be as defined in SEQ ID NO: 9 to 16 by the regions separating the CDRs (FR1, CDR1, FR2, CDR2, FR3, CDR3 and FR4):

H-FR1:
(SEQ ID NO: 9)
QVKLVESGGGVVQPGRSLTLSCAASGFTFS;
H-FR2:
(SEQ ID NO: 10)
WIRQAPGKGLEWIA;
H-FR3:
(SEQ ID NO: 11)
RFTISRDNSKNTLYLQMNSLRAEDTAVYYCAA;
H-FR4:
(SEQ ID NO: 12)
WGQGTMVTVSS;
L-FR1:
(SEQ ID NO: 13)
EIVLTQSPDFQSVTPKEKVTITCR;
L-FR2:
(SEQ ID NO: 14)
WYQQKPDQSPKLLIK;
L-FR3:
(SEQ ID NO: 15)
GVPSRFSGSGSGTDFTLTINRVEAEDAATYYC;
L-FR4:
(SEQ ID NO: 16)
FGAGTKLEIK.

In particular embodiments, the variable domain framework(s) of the antibody binding region that specifically binds a VEGF protein may be as defined in SEQ ID NO: 29 to 36 by the regions separating the CDRs (FR1, CDR1, FR2, CDR2, FR3, CDR3 and FR4):

H-FR1:
(SEQ ID NO: 29)
EVQLVESGGGLVQPGGSLRLSCAAS;
H-FR2:
(SEQ ID NO: 30)
WVRQAPGKGLEWVG;
H-FR3:
(SEQ ID NO: 31)
RFTFSLDTSKSTAYLQMNSLRAEDTAVYYCAK;
H-FR4:
(SEQ ID NO: 32)
WGQGTLVTVSS;
L-FR1:
(SEQ ID NO: 33)
DIQMTQSPSSLSASVGDRVTITC;
L-FR2:
(SEQ ID NO: 34)
WYQQKPGKAPKVLIY;
L-FR3:
(SEQ ID NO: 35)
GVPSRFSGSGSGTDFTLTISSLQPEDFATYYC;
L-FR4:
(SEQ ID NO: 36)
FGQGTKVEIKR.

Optionally, said antigen binding region that specifically binds a sCD146 protein and said antigen binding region that specifically binds a VEGF protein are covalently linked, especially fused together by a peptide linker.

In another aspect, the present invention relates to a nucleic acid or set of nucleic acids encoding an antibody of the invention as described above, or complementary to said encoding sequence. Preferably, the nucleic acid or set of nucleic acids is/are isolated or purified nucleic acid(s).

The terms “nucleic acid” and “polynucleotide” are used herein interchangeably. A nucleic acid of the invention can be DNA (cDNA or gDNA), RNA, or a mixture of the two. It can be in single stranded form or in duplex form or a mixture of the two. It can comprise modified nucleotides, comprising for example a modified bond, a modified purine or pyrimidine base, or a modified sugar. It can be prepared by any method known to one skilled in the art, including chemical synthesis, recombination, and mutagenesis. A nucleic acid according to the invention may be deduced from the sequence of the antibody according to the invention and codon usage may be adapted according to the host cell in which the nucleic acid shall be transcribed. These steps may be carried out according to methods well known to one of skill in the art and some of which are described in the reference manual Sambrook et al. (Sambrook J, Russell D (2001) Molecular cloning: a laboratory manual, Third Edition Cold Spring Harbor).

The nucleic acid or set of nucleic acids of the invention may encode an amino acid sequence comprising the light chain and/or an amino acid sequence comprising the heavy chain of the antibody, or may be complementary to such encoding sequence.

All embodiments related to the antibody of the invention are also contemplated in this aspect.

In particular, the description provides a nucleic acid molecule or set of nucleic acid molecules encoding:
i) an antibody comprising an antigen binding region that specifically binds a soluble CD146 protein (sCD146), wherein said antibody comprises a light chain variable region (VL) comprising the sequence of SEQ ID NO: 1 and a heavy chain variable region (VH) comprising the sequence of SEQ ID NO: 2, such as mucizumab,
ii) a multispecific antibody as herein described, or
iii) a combination of a) an antibody as defined in i), an antibody comprising one or more of the CDRs, VL and/or VH sequences specifically recognizing a sCD146 protein (i.e., comprising an antigen binding region that specifically binds a sCD146 protein) as herein described, or variant(s) thereof, with b) an antibody comprising one or more of the CDRs, VL and/or VH sequences specifically recognizing a VEGF protein (i.e., comprising an antigen binding region that specifically binds a VEGF protein) as herein described or variant(s) thereof, or bevacizumab antibody (CAS number 216974-75-3); or sequence(s) complementary to any one of the encoding sequence(s) described in i), ii) and iii).

A particular set of nucleic acids comprises a first nucleic acid molecule encoding a first antibody directed against a soluble CD146 protein as herein described and a second nucleic acid molecule encoding a second antibody directed against VEGF as herein defined.

In a further aspect, the present invention relates to a vector comprising a nucleic acid or set of nucleic acids of the invention. Optionally, the vector may comprise several nucleic acids of the invention. In particular, the vector may comprise a nucleic acid of the invention operably linked to a regulatory region, i.e. a region comprising one or more control sequences. Optionally, the vector may comprise several nucleic acids of the invention operably linked to several regulatory regions.

The term “control sequences” means nucleic acid sequences necessary for expression of a coding region. Control sequences may be endogenous or heterologous. Well-known control sequences and currently used by the person skilled in the art will be preferred. Such control sequences include, but are not limited to, promoter, signal peptide sequence and transcription terminator.

The term “operably linked” means a configuration in which a control sequence is placed at an appropriate position relative to a coding sequence, in such a way that the control sequence directs expression of the coding region.

All embodiments related to the antibody or the nucleic acid of the invention are also contemplated in this aspect.

In another aspect, the present invention further relates to the use of a nucleic acid or set of nucleic acids or vector according to the invention to transform, transfect or transduce a host cell. The present invention also provides a (host) cell comprising one or several nucleic acid molecules (also herein simply identified as “nucleic acids”) or set of nucleic acids of the invention and/or one or several vectors of the invention.

All embodiments related to the antibody, the nucleic acid, set of nucleic acids or the vector of the invention are also contemplated in this aspect.

The term “host cell” also encompasses any progeny of a parent host cell that is not identical to the parent host cell due to mutations that occur during replication.

Suitable host cells for cloning or expression of antibody-encoding vectors include prokaryotic or eukaryotic cells described herein.

For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli) After expression, the antibody may be isolated from the bacterial cell lysate in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern (cf. Gemgross, Nat. Biotech. 22: 1409-1414 (2004), and Li et al., Nat. Biotech. 24:210-215 (2006)).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (cf, e.g., U.S. Pat. Nos. 5,959,177, 6,040,498, 6,420,548, 7,125,978, and 6,417,429).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251(1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

In another aspect, the present invention also concerns a method for producing an antibody of the invention, comprising culturing a host cell comprising a nucleic acid or set of nucleic acids of the invention or a vector of the invention, as provided above, under conditions suitable for expression of the antibody, and optionally recovering the antibody from the host cell or from the host cell culture medium. Optionally, the recovered antibody may be further purified or isolated. Suitable media, culture conditions and production method are well-known by the skilled person and can be easily chosen according to the host cell and the antibody to be produced.

All embodiments related to the antibody, the nucleic acid or set of nucleic acids, the vector or the host cell of the invention are also contemplated in this aspect.

Methods for making multispecific antibodies and in particular bispecific antibodies may vary according to the format of the antibody and are well-known by the skilled person, see e.g. cf. Brinkmann and Kontermann, MAbs. 2017 February-March; 9(2): 182-212.

Once expressed, the antibodies of the invention, e.g. whole antibodies, their dimers, individual light and heavy chains, or other immunoglobulin forms of the present invention, can be further isolated or purified to obtain preparations that are substantially homogeneous for further assays and applications. Standard protein purification methods known in the art can be used. For example, suitable purification procedures may include fractionation on immunoaffinity or ion-exchange columns, ethanol precipitation, high-performance liquid chromatography (HPLC), sodium dodecyl sulfate polyacrylamide gel electrophoresis (SDS-PAGE), ammonium sulfate precipitation, and gel filtration. Substantially pure immunoglobulins of at least about 90 to 95% homogeneity are preferred, and 98 to 99% or more homogeneity most preferred, for pharmaceutical uses.

The herein described biological tools or products of the invention (antibodies, nucleic acid molecules, vectors, cells and compositions comprising the same) can be used as medicaments.

The herein described biological tools or products of the invention (antibodies, nucleic acid molecules, vectors, cells and compositions comprising the same) can be used in particular for preventing or treating cancer, for example a glioblastoma as herein described.

In a particular aspect, the present invention relates to a composition, in particular a pharmaceutical composition comprising an antibody, a nucleic acid or set of nucleic acids, a vector or a host cell of the invention. The composition may comprise one or several antibodies of the invention, one or several nucleic acid or set of nucleic acids of the invention and/or one or several vectors of the invention and/or one or several host cells of the invention. Preferably, the pharmaceutical composition comprises one or several antibodies of the invention.

All embodiments related to the antibody, the nucleic acid or set of nucleic acids, the vector or the host cell of the invention are also contemplated in this aspect.

A particular pharmaceutical composition, is herein described by inventors. This composition comprises i) a first antibody comprising an antigen binding region that specifically binds a sCD146 protein and second antibody comprising an antigen binding region that specifically binds a VEGF protein, preferably the VEGF-A isoform, wherein the second antibody comprises SEQ ID NO: 23 (H-CDR1), SEQ ID NO: 24 (H-CDR2), SEQ ID NO: 25 (H-CDR3), SEQ ID NO: 26 (L-CDR1), SEQ ID NO: 27 (L-CDR2) and SEQ ID NO: 28 (L-CDR3), or ii) a multispecific antibody, a nucleic acid molecule or set of nucleic acids, a vector and/or a (host) cell as herein described, and a pharmaceutically acceptable excipient or support.

Pharmaceutical compositions comprising one or several antibodies of the invention can be formulated according to known methods to prepare pharmaceutically useful compositions, whereby the antibody having the desired degree of purity is mixed with optional physiologically acceptable carriers, excipients or stabilizers (Remington: The Science and Practice of Pharmacy 20th edition (2000)), in the form of aqueous solutions, lyophilized or other dried formulations.

As used herein, the term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. Preferably, such formulations are sterile, i.e. aseptic or free from all living microorganisms and their spores.

Acceptable carriers, excipients, or stabilizers are nontoxic to recipients at the dosages and concentrations employed, and include, but are not limited to, buffering agents, stabilizing agents, preservatives, isotonifiers, non-ionic detergents, antioxidants and other miscellaneous additives.

Buffering agents help to maintain the pH in the range which approximates physiological conditions. Suitable buffering agents for use with the present invention include, but are not limited to, both organic and inorganic acids and salts thereof such as citrate, succinate, tartrate, fumarate, gluconate, oxalate, lactate and acetate buffers, as well as phosphate buffers, histidine buffers and trimethylamine salts such as Tris.

Preservatives may be added to retard microbial growth. Suitable preservatives for use with the present invention include, but are not limited to, phenol, butyl or benzyl alcohol; meta-cresol; alkyl parabens such as methyl or propyl paraben; octadecyldimethylbenzyl ammonium chloride, benzalkonium halides (e.g., chloride, bromide, iodide); hexamethonium or benzethonium chloride; catechol; resorcinol; cyclohexanol; and 3-pentanol.

Isotonifiers may be added to ensure isotonicity of liquid compositions of the present invention and include polhydric sugar alcohols, preferably trihydric or higher sugar alcohols, such as glycerin, erythritol, arabitol, xylitol, sorbitol and mannitol.

Stabilizing agents refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, alpha.-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran.

Non-ionic surfactants or detergents (also known as “wetting agents”) may be added to help solubilize the therapeutic agent as well as to protect the therapeutic protein against agitation-induced aggregation. Suitable non-ionic surfactants include, but are not limited to, polysorbates (20, 80, etc.), polyoxamers (184, 188 etc.), PLURONICS™, polyols, polyoxyethylene sorbitan monoethers (TWEEN™-20, TWEEN™-80, etc.).

Additional miscellaneous excipients include, but are not limited to, bulking agents, (e.g. starch), chelating agents (e.g. EDTA), antioxidants (e.g., ascorbic acid, methionine, vitamin E), and cosolvents.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin micropheres, microemulsions, nano-particles and nanocapsules) or in macroemulsions. Such techniques are disclosed in Remington: The Science and Practice of Pharmacy 20th edition (2000).

The form of the pharmaceutical compositions, the route of administration, the dosage and the regimen depend upon the condition to be treated, the severity of the illness, the age, weight, and sex of the patient, etc. The doses used for the administration can be adapted as a function of various parameters, and in particular as a function of the mode of administration used, of the relevant pathology, or alternatively of the desired duration of treatment.

The pharmaceutical compositions of the invention can be formulated for a topical, oral, parenteral, intranasal, intravenous, intra-tumoral, intrathecal, intramuscular, subcutaneous or intraocular administration and the like, preferably for an intravenous administration.

For the prevention or treatment of glioblastoma, preferred administration routes are intravenous, intrathecal and subcutaneous routes, even more preferably intravenous and intrathecal routes.

Preferably, the pharmaceutical formulation is a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Sterile injectable solutions may be prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the compositions formulated for parenteral administration, such as intravenous or intramuscular injection, other pharmaceutically acceptable forms include, e.g. tablets or other solids for oral administration; time release capsules; and any other form currently used.

The pharmaceutical composition of the invention may comprise one or several antibodies of the invention.

A particular pharmaceutical composition comprises at least one antibody directed against a sCD146 protein, said first antibody comprising a light chain variable region (L) and a heavy chain variable region (H) comprising the following combination of 6 CDRs:
H-CDR1 of SEQ ID NO: 3 or a variant thereof,
H-CDR2 of SEQ ID NO: 4 or a variant thereof
H-CDR3 of SEQ ID NO: 5 or a variant thereof, and
L-CDR1 of SEQ ID NO: 6 or a variant thereof,
L-CDR2 of SEQ ID NO: 7 or a variant thereof,
L-CDR3 of SEQ ID NO: 8 or a variant thereof, said variants having, as explained previously, at least 80%, preferably at least 85%, at least 90%, at least 95%, at least 96%, at least 97%, at least 98% or at least 99% sequence identity to the recited CDR sequences

A particular pharmaceutical composition comprises at least one antibody directed against a sCD146 protein, said at least one antibody comprising a light chain variable region (VL) comprising the sequence of SEQ ID NO: 1 or variant thereof and a heavy chain variable region (VH) comprising the sequence of SEQ ID NO: 2, or a variant thereof, said variants having, as explained previously, at least 80%, preferably at least 85%, at least 90%, at least 95%, at least 96%, at least 97%, at least 98% or at least 99% sequence identity to the recited sequences.

Another particular pharmaceutical composition comprises at least one antibody directed against a VEGF protein, said first antibody comprising a light chain variable region (L) and a heavy chain variable region (H) comprising the following combination of 6 CDRs:
H-CDR1 of SEQ ID NO: 23 or a variant thereof,
H-CDR2 of SEQ ID NO: 24 or a variant thereof,
H-CDR3 of SEQ ID NO: 25 or a variant thereof, and
L-CDR1 of SEQ ID NO: 26 or a variant thereof,
L-CDR2 of SEQ ID NO: 27 or a variant thereof,
L-CDR3 of SEQ ID NO: 28 or a variant thereof, said variants having at least 80%, preferably at least 85%, at least 90%, at least 95%, at least 96%, at least 97%, at least 98% or at least 99% sequence identity to the recited CDR sequences.

A further particular pharmaceutical composition comprises at least one antibody directed against a VEGF protein, said at least one antibody comprising a light chain variable region (VL) comprising the sequence of SEQ ID NO: 37 or a variant thereof and a heavy chain variable region (VH) comprising the sequence of SEQ ID NO: 38, or a variant thereof, said variants having, as explained previously, at least 80%, preferably at least 85%, at least 90%, at least 95%, at least 96%, at least 97%, at least 98% or at least 99% sequence identity to the recited sequences.

Another particular pharmaceutical composition comprises a first antibody directed against a sCD146 protein said first antibody comprising a light chain variable region (L) and a heavy chain variable region (H) comprising the combination of CDR sequence SEQ ID NO: 3 to 8 (or any alternative combination including variant(s) thereof) and/or said first antibody comprising a light chain variable region (VL) and a heavy chain variable region (VH) comprising the combination sequence SEQ ID NO: 1 and 2 (or any alternative combination including variant(s) thereof) respectively, and a second antibody directed against a VEGF protein said second antibody comprising a light chain variable region (L) and a heavy chain variable region (H) comprising the combination of CDR sequence SEQ ID NO: 23 to 28 (or any alternative combination including variant(s) thereof) and/or said second antibody comprising a light chain variable region (VL) and heavy chain variable region (VH) comprising the combination sequence 37 and 38 (or any alternative combination including variant(s) thereof) respectively, wherein said variants have at least 80%, preferably at least 85%, at least 90%, at least 95%, at least 96%, at least 97%, at least 98% or at least 99% sequence identity to the recited sequences.

Any one of the herein described pharmaceutical composition may further comprise one or several additional active compounds. Examples of additional active compounds include, but are not limited to any distinct known anti-angiogenic or immune checkpoint inhibitor(s), in particular antibody(ies).

In a further aspect, the present invention relates to an antibody of the invention or a pharmaceutical composition of the invention for use for treating a disease or condition condition wherein the ill subject does not respond (i.e., is resistant) to treatments involving an anti-VEGF compound or any condition (/pathology) for which resistance to treatment can be shown by an increase in the level of sCD146 in a fluid biological sample of the subject. The present invention also relates to the use of antibody(ies) or a pharmaceutical composition of the invention for the manufacture of a medicament to a disease caused or exacerbated by excessive angiogenesis, such as cancer and age-related macular degeneration (ARMD or RMD). The present invention further relates to a method of treating a disease caused or exacerbated by excessive angiogenesis, such as cancer and ARMD, in a subject, comprising administering to a subject suffering from said disease an effective amount of the antibody(ies) or pharmaceutical composition of the invention. All embodiments related to the antibody, the nucleic acid or set of nucleic acids, the vector, the host cell or the pharmaceutical composition of the invention are also contemplated in this aspect.

The antibody herein described, including the multispecific antibodies, the nucleic acid molecule or set of nucleic acids, the vector, the (host) cell, as well as the pharmaceutical composition, are herein described for use for preventing or treating any condition wherein the ill subject does not respond (i.e., is resistant) to treatments involving an anti-VEGF compound or any condition (/pathology) for which resistance to treatment can be shown by an increase in the level of sCD146 in a fluid biological sample of the subject. The condition is typically a cancer, preferably a cancer the conventional treatment of which involves an anti-VEGF compound such as bevacizumab, for example glioblastoma, typically a glioblastoma resistant to anti-VEGF treatment or a glioblastoma whose cells express membranous CD146 (i.e., a CD146+ glioblastoma).

The antibody herein described, including the multispecific antibodies, the nucleic acid molecule or set of nucleic acids, the vector, the (host) cell, as well as the pharmaceutical composition, are in particular herein described for use for treating a subject suffering of cancer who has been, who is, or who will be treated with bevacizumab, or for treating a subject identified as, or known to be, resistant to bevacizumab treatment.

The amount of (each) antibody of the invention which will be effective in the treatment of a particular disorder or condition will depend on the nature of the disorder or condition, and can be determined by standard clinical techniques.

The effective amount may be a therapeutically or prophylactically effective amount. A “therapeutically effective amount” refers to an amount effective, at dosages and for periods of time necessary, to achieve the desired therapeutic or prophylactic result. The therapeutically effective amount of an antibody or composition of the invention may vary according to factors such as the disease state, age, sex, and weight of the individual, and the ability of the antibody or composition, to elicit a desired response in the individual. A therapeutically effective amount encompasses an amount in which any toxic or detrimental effects of the antibody or composition are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

In a preferred embodiment, each dose may range from 0.1 to 1,000 mg per kilogram of body weight of antibody, or more preferably, from 10 to 30 mg per kilogram body weight.

The dosing schedule for administration may vary form once a month to daily depending on a number of clinical factors, including the type of disease, severity of disease, and the subject's sensitivity to the therapeutic agent.

In the methods and uses of the present invention, the antibody(ies) of the invention may be used in combination with other active ingredients that can be chosen according to the disease to be prevented or treated. Examples of other active ingredients include, but are not limited to, any distinct known anti-angiogenic, or immune checkpoint inhibitor(s), in particular antibody(ies). Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant.

An example of additional therapeutic agents is for example carboplatine.

An antibody of the invention (and any additional therapeutic agent) can be administered by any suitable means, including parenteral, intrapulmonary, and intranasal, and, if desired for local treatment, intralesional, for example intra-tumoral, administration. Parenteral infusions include intramuscular, intravenous, intraarterial, intraperitoneal, or subcutaneous administration. Dosing can be by any suitable route, e.g. by injections, such as intravenous, intrathecal, or subcutaneous injections, depending in part on whether the administration is brief or chronic. Various dosing schedules including but not limited to single or multiple administrations over various time-points, bolus administration, and pulse infusion are contemplated herein.

In another aspect, the present invention also relates to an antibody(ies) or a pharmaceutical composition of the invention for use in a method of reducing, inhibiting or preventing tumor growth. It further relates to a method of reducing, inhibiting or preventing tumor growth comprising administering to the subject an effective amount of antibody(ies) or a pharmaceutical composition of the invention. It also relates to the use of an antibody(ies) or a pharmaceutical composition of the invention for the manufacture of a medicament to reduce, inhibit or prevent tumor growth.

Tumor growth can be assessed by any method known by the skilled person such as PET imaging, as disclosed in the experimental section.

All embodiments related to the antibody, the nucleic acid, the vector, the host cell or the pharmaceutical composition of the invention are also contemplated in this aspect.

In the context of the present invention, the cancer or tumor is a cancer or malignant tumor resistant to cancer treatment involving an anti-VEGF. The cancer is typically a cancer conventionally treated with an anti-VEGF agent.

In a preferred aspect, the anti-VEGF agent is an anti-VEGF antibody, typically an anti-VEGF-A antibody such as bevacizumab.

The cancer is preferably selected from a glioblastoma, a renal cell carcinoma, a melanoma, a breast cancer, in particular a triple-negative breast cancer, and a pancreatic cancer.

In a preferred embodiment, the cancer is a glioblastoma, preferably a glioblastoma multiform (GBM), in particular a CD146+ glioblastoma.

Glioblastoma multiforme (GBM) is the most common malignant primary brain tumor with extremely poor prognosis because of its diffusive and infiltrative nature. It accounts for more than 50% of all gliomas (Weller M. et al., 2015). Despite decades of research, GBM remains among the most lethal of all forms of cancers. The five-year survival rate barely reaches 3% in patients and the median survival of patients is about 14 months, even after first line treatment including surgery, radiotherapy and chemotherapy. Resistance to this conventional line of treatment is systematic and results in tumor recurrence (Osuka S. et al., 2017). Vascular proliferation that is markedly powered by Vascular Endothelial Growth Factor (VEGF) signaling is a hallmark in GBM. Bevacizumab, a neutralizing monoclonal anti-VEGF antibody, has been recognized as a potent drug candidate in the treatment of glioblastoma at recurrence. Multiple phase III clinical trials including the AVAglio, RTOG 0825 and the EORTC 26101 have confirmed a significantly longer progression-free survival (PFS) in GBM patients treated with bevacizumab. However, bevacizumab has no effect on overall survival. Indeed, bevacizumab was able to diminish tumor volume, intratumoral angiogenesis and oxygen consumption in vivo at early cycles of treatment, which could explain the increase in PFS, but why this did not translate into overall survival benefit remains to be investigated (Bonekamp D., 2017).

Thanks to the present invention, inventors now herein provide a new therapeutic option to treat patients suffering of glioblastoma, in particular glioblastoma resistant to anti-VEGF treatments.

As used herein, the term “subject” or “patient” refers to a mammal, in particular a rodent such as a mouse or rat, a horse, a dog, a cat and a human being, preferably a human being, whatever its age or sex. The patient typically has a cancer or tumor. Unless otherwise specified in the present disclosure, the tumor is a cancerous or malignant tumor as herein above defined. In a particular aspect, the subject is i) a subject who has not been previously exposed to a treatment of cancer involving an anti-VEGF agent, in particular, when the cancer is a glioblastoma, to a treatment of glioblastoma involving an anti-VEGF antibody, or ii) a subject who has received at least a first administration of such a treatment of cancer, typically of an anti-VEGF agent as described herein above. In another preferred embodiment, the subject is a subject who has undergone at least partial resection of the cancerous tumor or a complete resection thereof.

Another particular subpopulation of subjects is composed of subjects having metastases, typically subjects suffering of glioblastoma having metastasis.

In another aspect, inventors herein describe the use (in particular in vitro or ex vivo) of a soluble CD146 protein as a biomarker for predicting or monitoring the response to a treatment involving an anti-VEGF agent, such as bevacizumab, of a subject suffering from cancer as herein described.

In a preferred aspect, the cancer is glioblastoma, preferably a sCD146+ glioblastoma.

In another preferred aspect, the anti-VEGF agent is bevacizumab.

They describe in particular an in vitro or ex vivo method for predicting the response to a treatment involving an anti-VEGF agent, such as bevacizumab, of a subject suffering from cancer comprising i) determining the soluble CD146 protein expression level in a biological sample of the subject before treatment, ii) comparing the expression level determined at step i) with a reference value, and iii) concluding that the subject is likely to respond to the treatment when the level determined at step i) is lower than the reference value, or concluding that the patient is unlikely to respond to the treatment when the level determined at step i) is higher than the reference value.

In a particular aspect, the reference value is between about 300 and 350 ng/ml of biological sample of the subject, and is preferably of about 310, 315, 320, 325, 330, 335, 340 or 345 ng/ml±5 ng/ml. For example, the reference value is of 320 ng/ml of the serum sample of the subject.

Predictive methods of the invention can be used clinically to make treatment decisions by choosing as soon as possible the most appropriate treatment modalities for a particular patient.

If the subject is identified, using a method according to the present invention, as resistant to a particular treatment of cancer, typically a treatment involving an anti-VEGF such as bevacizumab, the method advantageously further comprises a step of selecting a distinct treatment of cancer, typically involving a “compensatory molecule”, to be used instead of or in combination with the originally preselected drug (typically bevacizumab) or with a distinct drug, as the appropriate therapeutic treatment of cancer for the subject.

Preferably, the reference expression level of sCD146 is calculated from the expression level of sCD146 determined in a biological sample of the subject before any administration of a cancer treatment, typically of a treatment involving an anti-VEGF such as bevacizumab, in the subject, i.e. before any administration to the subject of a drug for treating the subject's cancer, preferably before any administration thereof to the subject. Less preferably but also possible, this step can be performed after the first or a subsequent administration of an anti-VEGF drug, preferably bevacizumab to the subject. This step can be performed before any tumor surgical resection. It can also be performed on a subject who has undergone at least partial resection of the cancerous tumor or complete resection thereof.

In a particular aspect, this step of determining the expression level of sCD146 in a biological sample of the subject is performed at least twice, a first time as described previously before any administration to the subject of a drug for treating the subject's cancer or after the first or a subsequent administration of such a drug, and at least a second time after either the first or subsequent (for example second or third) administration thereof in order to be able to compare the measured expression levels.

In preferred aspect, this step of determining the expression level of sCD146 in a biological sample of the subject is performed only once, the determined level being then compared to a reference sCD146 level.

Typically, the “reference value” or “reference expression level” is the level of sCD146 in a control sample derived from one or more subjects (reference population) having a cancer, in particular a glioblastoma, and is typically the median value when obtained from the reference population.

The measured sCD146 expression level, preferably the first measured sCD146 expression level, typically the sCD146 expression level measured at diagnosis of the cancer of the subject (before any cancer treatment of the subject, in particular a treatment involving anti-VEGF agent such as bevacizumab), is also typically usable to determine or calculate a sCD146 reference expression level which can then be compared to the measured sCD146 expression level.

In a preferred embodiment, the sCD146 reference expression level is calculated from the sCD146 expression level determined in a biological sample from the subject before any administration of anti-VEGF agent such as bevacizumab to said subject.

In a particular embodiment, the herein described in vitro or ex vivo method of assessing, typically of determining, of predicting or of monitoring, the sensitivity of a subject having a cancer or tumor to a cancer treatment, in particular an anti-VEGF agent, preferably anti-VEGF-A agent, such as bevacizumab, comprises a step of determining, typically measuring, in a biological sample from said subject, the expression level of sCD146 after treatment of the subject with the cancer treatment, in particular after a first or subsequent treatment of the subject with the cancer treatment, or in a particular embodiment after a first or subsequent cycle of treatment with the cancer treatment, and typically a step of comparing said sCD146 expression level to a sCD146 reference expression level or reference value, thereby assessing whether the subject having a cancer or tumor is responsive or resistant to the cancer treatment, a sCD146 expression level identical to or above the sCD146 reference expression level, being the indication that the subject is resistant to the cancer treatment, in particular to the anti-VEGF agent such as bevacizumab, when used alone (i.e. as single active molecule); and, on the contrary, a sCD146 expression level below the sCD146 reference expression level being the indication that the subject is sensitive to the cancer treatment, in particular the anti-VEGF agent such as bevacizumab, even when used alone.

In a preferred embodiment where bevacizumab is specifically used as the cancer treatment, the sCD146 reference expression level is equal to the sCD146 expression level, as determined in a biological sample from the subject before any administration of bevacizumab to said subject, to which is added (plus) 20% of said sCD146 expression level (determined in the biological sample from the subject before any administration of bevacizumab).

Inventors herein demonstrate that a sCD146 expression level below the sCD146 reference expression level is indicative of a sensitivity of the subject, typically of a subject suffering of a renal cancer as herein described, to bevacizumab, and a sCD146 expression level identical to or above said reference expression level is indicative of a resistance of the subject, typically of a subject suffering of a glioblastoma as herein described, to bevacizumab.

Inventors also describe in particular an in vitro or ex vivo method for monitoring the response, or sensitivity, to a treatment involving an anti-VEGF agent, such as bevacizumab, of a subject suffering from cancer, in particular glioblastoma, comprising determining, typically measuring, the soluble CD146 protein level of expression in a biological sample of said subject at two or more time points during said treatment, wherein a higher soluble CD146 protein level of expression in a biological sample of the subject at a later time point, compared to a reference value obtained in a biological sample of the subject at an earlier time point, is indicative of a resistance of the subject to said treatment whereas an equal or lower soluble CD146 protein level is indicative of a response of the subject to said treatment.

In a particular aspect, the reference value of soluble CD146 protein level of expression is determined before the treatment involving an anti-VEGF agent such as bevacizumab, of the subject.

In such a monitoring method, the treatment of the subject with the anti-VEGF agent such as bevacizumab is typically a second, third, or a subsequent treatment step with such an agent.

In a particular embodiment, each measured sCD146 expression level is typically to be compared with the measure directly preceding the measure under consideration or with several of said preceding measures when existing.

In a preferred embodiment, each measured sCD146 expression level is typically to be compared with the measure performed before any treatment with, or any administration of, the anti-VEGF agent.

When the treatment is identified as not effective or not efficient enough, a method including a step, as herein above described, of selecting an appropriate, preferably optimal, alternative therapeutic treatment of cancer will be advantageously performed.

In the methods herein described of assessing or predicting the sensitivity of a subject having a tumor to a cancer treatment, any classical method known by the skilled person of determining the presence or measuring the expression level of a compound of interest, typically sCD146, can be performed.

Preferably, the determination of the presence as well as the measure of the quantities of sCD146, is determined in an immunoassay through a one-step method wherein the subject's biological sample is directly contacted with the appropriate ligand or through a method implying a preliminary treatment of the biological sample. An immunoassay can typically be performed through well-known methods of the art: in solid phase or homogeneous phase, in one or two steps, through competitive method, etc. The immunoassay is typically selected from the group consisting of ELISA, FEIA, multiplex, western blot, dot blot, bead-based assay, antigen array and Radio Immuno Assay.

In an ELISA, an antigen must be immobilized to a solid surface and then complexed with an antibody that is linked to an enzyme. Detection is accomplished by assessing the conjugated enzyme activity via incubation with a substrate to produce a colored product.

In FEIA, the colored product is fluorescent.

In Radio Immuno Assay, the final product is radioactive.

Protein detection using the dot blot protocol is similar to western blotting in that both methods allow for the identification and analysis of proteins of interest. Dot blot methodology differs from traditional western blot techniques by not separating protein samples using electrophoresis. Sample proteins are instead spotted onto membranes and hybridized with an antibody probe. Bead-based assay or antigen array are new approaches for investigators to simultaneously measure multiple analytes in biological and environmental samples.

Semi-quantitative measurements can be obtained with each of the previously described methods using for example normal controls to normalize the value and then establish a ratio, or using a positive control as a calibrator (expressed in arbitrary units).

The detection may be performed on a solid support, for example a microplaque, or solid particles, test tubes, etc.

Preferably, the selected immunoassay is an ELISA or radioimmunoassay.

The present invention in particular covers methods involving using an ELISA assay to identify a sCD146 protein. In some embodiments, the ELISA assay is a sandwich assay. In a sandwich assay, more than one antibody will be employed. Typically ELISA method can be used, wherein the wells of a microtiter plate are coated with a set of antibodies which recognize the protein of interest. A sample containing or suspected of containing the protein of interest is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In some embodiments of the invention, identification of sCD146 involves use of at least one sCD146 binding agent, typically polypeptide binding agent. A preferred sCD146 binding agent is capable of selectively binding soluble CD146 (versus membrane CD146). The sCD146 binding agent is, in particular embodiments, an antibody, for example a synthetic, monoclonal or polyclonal antibody, in particular an anti-sCD146 antibody capable of selectively binding soluble CD146 (versus membrane CD146). Preferably, the anti-sCD146 antibody is selected from a specific anti-sCD146 antibody of the art. This antibody can be bi-specific, recognizing two different epitopes. The antibody, in some embodiments, immunologically binds to more than one epitope from the same sCD146 polypeptide. A sCD146 polypeptide binding agent that is a polypeptide may also include all or part of a receptor for sCD146 polypeptides, such as angiomotin (Amot). A particular sCD146 binding agent usable in the context of the invention is capable of binding both the human soluble CD146 protein and a CD146 or soluble CD146 protein receptor (or a CD146 protein receptor subunit).

The antibody binding sCD146 can be made by techniques well known in the art. Methods of making such antibodies are known in the art (see for example DespoixNb et al., 2008).

In preferred embodiments, the anti-sCD146 antibody is a monoclonal antibody. A monoclonal antibody selectively binding sCD146 (versus membrane CD146), usable in the context of the invention, is advantageously selected from clone COM 3D9, clone COM 2F6, clone COM 5G6, clone COM 7A4 and clone F4-35H7 (S-endo 1) from BioCytex, and is preferably clone COM 7A4.

Antibodies usable in the context of the present invention may also be anti-antibodies used to identify, follow, detect and/or measure the antibodies that recognize the sCD146.

In some embodiments of the invention, the soluble CD146 binding agent is an aptamer.

In some embodiments of the invention, the sCD146 binding agent, typically antibody or aptamer, is labelled with one or more tags (detection agent, marker or label) allowing for their identification, follow-up, detection and/or measurement. The detection agent, marker or label may be selected for example from a fluorophore, a chemiluminescent particle, a radioisotope, a magnetic bead, an antigenic epitope, an enzyme (such as horseradish peroxidase), a substrate of a specific enzyme, a ligand or binding domain of sCD146, and any other molecule or moiety which may be detected or quantified. A detection agent may be an antibody that binds to a sCD146 polypeptide binding agent, such as an antibody. The detection agent antibody, in some embodiments, binds to the Fc-region of a binding agent antibody.

It is also specifically contemplated that a binding agent is unlabelled, but may be used in conjunction with a detection agent that is labelled. A detection agent is a compound that allows for the detection or isolation of itself so as to allow detection of another compound that binds, directly or indirectly. An indirect binding refers to binding among compounds that do not bind each other directly but associate or are in a complex with each other because they bind the same compounds or compounds that bind each other.

Other embodiments of the invention involve a second sCD146 binding agent in addition to a first sCD146 binding agent. The second binding agent may be any of the entities discussed above with respect to the first binding agent, such as an antibody. It is contemplated that a second antibody may bind to the same or different epitopes as the first antibody. It is also contemplated that the second antibody may bind the first antibody or another epitope than the one recognized by the first antibody.

In a particular aspect, the soluble CD146 protein level of expression is determined by measuring the concentration of soluble CD146 protein in a liquid biological sample of the subject, preferably in a plasma sample.

Implementations of the methods of the invention involve obtaining a (biological) sample from a subject. The sample is preferably a fluid sample and may be selected for example from blood, typically total blood, serum, plasma, urine, lymphatic fluid, spinal fluid, pleural effusion, ascites, and a combination thereof. The sample is typically selected from a blood sample, a serum sample, a plasma sample, a urine sample or a derivative thereof. A particular sample is a plasma sample or a derivative thereof such as a plasma sample free of (or devoid of) blood platelets. Another preferred sample is a fluid sample without cells. The biological sample is preferably a diluted sample, the dilution being typically of 1/50, 1/100, 1/200 or 1/400.

In a particular aspect, a method for treating a subject suffering of a cancer identified (thanks to a method as herein described) as resistant to treatments involving an anti-VEGF agent, such as bevacizumab, is herein described. The cancer is for example a glioblastoma, in particular a CD146+ glioblastoma. The method typically comprises the administration of an antibody as herein described, in particular a multispecific or bispecific antibody as herein described. When different antibodies are administered, they are either simultaneously or sequentially administered to the subject in need thereof.

The present invention also includes kits for assessing or monitoring in vivo, in vitro or ex vivo the sensitivity or resistance of a subject having a cancer, preferably a glioblastoma, to a treatment involving an anti-VEGF agent such as bevacizumab.

In a particular embodiment, the kit comprises detection means, possibly in suitable container means, preferably at least one, for example two, sCD146 polypeptide binding agent(s) (typically antibody(ies) specific to sCD146, typically antibody(ies) which do(es) not bind membranous CD146); optionally a molecule allowing the binding agent(s) (typically antibody(ies)) detection; and, optionally, a leaflet providing the sCD146 reference expression level in a biological sample from a control or reference population or the formula for calculating the sCD146 reference expression level or threshold.

In further embodiments, the binding agent is labelled or a detection agent is included in the kit. It is contemplated that the kit may include a sCD146 polypeptide binding agent(s) attached to a non-reacting solid support, such as a tissue culture dish or a plate with multiple wells. It is further contemplated that such a kit includes at least one, for example two, detectable agent(s) in certain embodiments of the invention. In some embodiments, the invention concerns kits for carrying out a method of the invention comprising, in suitable container means: (a) an agent that specifically recognizes all or part of a sCD146 polypeptide, preferably an agent that specifically recognizes all or part of a sCD146 polypeptide; and, (b) positive control(s) that can be used to determine whether the agent is capable of specifically recognizing all or part of a sCD146 polypeptide. The kit may also include other reagents that allow visualization or other detection of the sCD146 polypeptide, such as reagents for colorimetric or enzymatic assays. Generally, the kit comprises one or more containers filled with one or more of the herein described products.

A labelling notice providing instructions for using the products in the context of a method according to the present invention can further be provided.

The present invention further relates to the use of a kit as herein described i) for assessing or monitoring in vivo, in vitro or ex vivo the sensitivity or resistance of a subject having a cancer, in particular a glioblastoma, to a treatment involving an anti-VEGF agent such as bevacizumab, and/or ii) for determining in vivo, in vitro or ex vivo the potential toxicity of said anti-VEGF agent for a subject having a cancer, in particular a glioblastoma. Preferably, the kit comprises detection means comprising at least one sCD146 binding agent and a molecule allowing the binding agent detection.

The following examples are given for purposes of illustration and not by way of limitation.

### Examples

The invention is illustrated below with the treatment of glioblastoma (also known as glioblastoma multiforme, “GBM”). Glioblastoma multiforme (GBM) is the most common malignant primary brain tumor with extremely poor prognosis because of its diffusive and infiltrative nature. It accounts for more than 50% of all gliomas (Weller M. et al., 2015). Despite decades of research, GBM remains among the most lethal of all forms of cancers. The five-year survival rate barely reaches 3% in patients and the median survival of patients is about 14 months, even after first line treatment including surgery, radiotherapy and chemotherapy. Resistance to this conventional line of treatment is systematic and results in tumor recurrence (Osuka et al., 2017). Vascular proliferation that is markedly powered by Vascular Endothelial Growth Factor (VEGF) signaling is a hallmark in GBM. Bevacizumab, a neutralizing monoclonal anti-VEGF antibody, has been recognized as a potent drug candidate in the treatment of glioblastoma at recurrence. Multiple phase III clinical trials including the AVAglio, RTOG 0825 and the EORTC 26101 have confirmed a significantly longer progression-free survival (PFS) in GBM patients treated with bevacizumab. However, bevacizumab has no effect on overall survival. Indeed, bevacizumab was able to diminish tumor volume, intratumoral angiogenesis and oxygen consumption in vivo at early cycles of treatment, which could explain the increase in PFS, but why this did not translate into overall survival benefit remains to be investigated (Bonekamp D. et al., 2017).

As described below, inventors found that an increase in sCD146 concentration in sera of GBM patients after the first cycle of bevacizumab treatment was significantly associated with poor progression free survival and shorter overall survival. Accordingly, in vitro treatment of CD146-positive glioblastoma cells with bevacizumab led to a high sCD146 secretion, inducing cell invasion. These effects were mediated through integrin αvβ3 and were blocked by mucizumab, a novel humanized anti-sCD146 antibody. In vivo, the use of a combination of bevacizumab with mucizumab, as well as the use of a bispecific antibody directed against both sCD146 and VEGF (i.e. a bispecific antibody comprising the light and heavy chain variable regions from mucizumab, and the light and heavy chain variable regions from bevacizumab), impeded CD146+ glioblastoma growth and reduced tumor cell dissemination to an extent significantly higher than that observed with bevacizumab alone or mucizumab alone.

### Materials and Methods

### Cell Culture

The three human glioblastoma cell lines U87, U118 and U373 were a kind gift from Dr. F. Peiretti (INSERM UMR-S 1263 Marseille, France). Cells were confirmed to be mycoplasma free. U118 and U373 cells were cultured in DMEM/F12 GlutaMAX™ supplemented with 10% heat inactivated FCS while U87 cells were cultured in DMEM (1 g/L glucose) enriched with 10% FCS. All media contained 100 U/ml penicillin, 100 μg/mL streptomycin and 2 mM L-glutamine. Cells were maintained in 5% CO₂ at 37° C. For the generation of bevacizumab resistant U87, U118 and U373, 2×10⁵ parental wild-type cells were cultured with 100 μg/mL of bevacizumab (Roche) in the culture media for up to 2 months. CHO cells were cultured in DMEM medium containing 10% FCS.

### Ethics Approval and Consent to Participate:

Plasma samples from patients with GBM were obtained from Assistance Publique-Höpitaux de Marseille Tumor Bank, authorization number AC2018-31053; CRB BB-0033-00097. Informed consent of patients was obtained. All animal experiments were conformed to the directive 2010/63/EU of the European Parliament and approved by the Institution's Animal care and Use Committee (Aix-Marseille University).

### Peptides and Antibodies

Recombinant human sCD146 (rsCD146) and VEGF-165 (rVEGF) were obtained from Biocytex (Marseille, France) and Invitrogen (PHC9391), respectively. Recombinant human integrin αvβ3 was from R&D (cat #3050-AV-050) and recombinant RGD peptide was from Santa Cruz (sc-201176). Antibodies used in this study are listed in table 1 below.

Cloning of the heavy and light chain variable domains in CrossMAb KIH MI-mAbs' expression vectors was performed. To this end, gene synthesis of the variable domains was done when necessary. PCR amplification was run using PrimeSTAR Max DNA Polymerase (Takara) and cloning of the PCR products was performed in expression vectors (2 for the light chain and 2 for the heavy chain). PCR purification was done using Nucleospin Gel and PCR Clean-up (Macherey Nagel), cloning by In-Fusion (Clontech) in transient expression vectors and transformation using Stellar competent cells (Clontech). Finally, vector sequencing was run by MWG Eurofins All these steps were performed by MiMabs company (Marseille, France).

TABLE 1
references and application condition of the different antibodies used in the study

| Antibody | Manufacturer | Reference | Dilution | Application |
|---|---|---|---|---|
| Phospho-VEGFR2 (Tyr1054), Clone D1W | Merck | 04-894 | 1:1000 | WB |
| Anti- VEGF Receptor 2 antibody, Clone A3 | Santa Cruz | sc-6251 | 1:1000 | WB |
| Anti-VEGF Receptor 2 antibody | Abcam | ab39638 | 1:1000 | WB |
| Phospho-FAK (Tyr397) | ThermoFisher | 44- | 1:1000 | WB |
| | | 624G | | |
| FAK Polyclonal Antibody | ThermoFisher | AHO0502 | 1:1000 | WB |
| Phospho-Integrin β3 Antibody (pY759.7A) | Santa Cruz | sc-136458 | 1:1000 | WB |
| Integrin β3 Antibody (D-11) | Santa Cruz | sc-365679 | 1:1000 | WB |
| Anti-human CD51 Antibody | Biolegend | 327902 | 1:750 | WB |
| CD51/CD61 Monoclonal Antibody (23C6) | eBioscience | 14-0519-82 | 5 μg | IP/ELISA |
| Anti-CD146 COM7A4 (human specific) | BioCytex | 5040-P | 1:750 | WB/ELISA |
| Anti-CD146, S-Endo1 | BioCytex | 5050-P | 5 μg | IP |
| Anti-CD146 antibody [EPR3208] | Abcam | ab75769 | 1:1000 | WB |
| Phospho-Tyr Antibody (PY99) | Santa Cruz | sc-7020 | 1:750 | WB |
| Phospho-Akt (Ser473) Antibody | CST | #9271 | 1:1000 | WB |
| Anti-Akt Antibody | CST | #9272 | 1:1000 | WB |
| Phospho-p38MAPK (Thr180/Tyr182) | CST | #9211 | 1:1000 | WB |
| Antibody | | | | |
| Anti-p38 MAPK Antibody | CST | #9212 | 1:1000 | WB |
| Phospho-ERK1/2 (Thr202/Tyr204) | BioLegend | 675502 | 1:1000 | WB |
| Antibody | | | | |
| Anti-ERK 1/2 Antibody (C-9) | Santa Cruz | sc-514302 | 1:1000 | WB |
| N-Cadherin (D4R1H) XP ® Rabbit mAb | CST | #13116 | 1:1000 | WB |
| E-Cadherin (24E10) Rabbit mAb | CST | #3195 | 1:1000 | WB |
| Vimentin (D21H3) XP ® Rabbit mAb | CST | #5741 | 1:2000 | WB |
| Snail (C15D3) Rabbit mAb | CST | #3879 | 1:1000 | WB |
| Slug (C19G7) Rabbit mAb | CST | #9585 | 1:1000 | WB |
| Nanog Antibody | R&D | AF1997 | 1:1000 | WB |
| Oct-3/4 Antibody (C-10) | Santa Cruz | sc-5279 | 1:1000 | WB |
| Sox-2 Antibody (A-5) | Santa Cruz | sc-365964 | 1:1000 | WB |
| β-Actin Antibody | CST | #4967 | 1:2000 | WB |
| Anti-EpCAM Antibody (C-10) | Santa Cruz | sc-25308 | 1:800 | WB |
| Anti-MAX antibody [73C5a] | Abcam | ab53570 | 1:1000 | WB |
| Anti-β-Amyloid, 1-16 Antibody | BioLegend | 803004 | 1:1000 | WB |
| Goat anti-Mouse IgG (H + L) Secondary | ThermoFisher | 31430 | 1:2500 | WB/ELISA |
| Antibody, HRP | | | | |
| Goat anti-Rabbit IgG (H + L) Secondary | ThermoFisher | 31460 | 1:2500 | WB |
| Antibody, HRP | | | | |
| Rabbit anti-Goat IgG (H + L) Secondary | ThermoFisher | 81-1620 | 1:2500 | WB |
| Antibody, HRP | | | | |
| FITC anti-human CD51/61 Antibody | BioLegend | 304404 | | FC |
| FITC Mouse IgG1, κ Isotype Ctrl Antibody | BioLegend | 400108 | | FC |
| APC anti-human CD146 Antibody | BioLegend | 361016 | | FC |
| APC Mouse IgG1, κ Isotype Ctrl (FC) | BioLegend | 400122 | | FC |
| Antibody | | | | |
| PE anti-human CD309 (VEGFR2) Antibody | BioLegend | 359904 | | FC |
| PE Mouse IgG1, κ Isotype Ctrl Antibody | BioLegend | 400112 | | FC |
| Angiomotin Antibody (B-4) FITC | Santa Cruz | sc-166924 | | FC |
| FITC Mouse IgG2b, κ Isotype Ctrl Antibody | BioLegend | 400310 | | FC |
| Human IgG1, whole molecule | Jackson | 009-000-003 | 5 mg/kg | In vivo |
| | ImmunoResearch | | | |
| | Laboratories | | | |
| Bevacizumab (Bvz) | Roche | | 5 mg/kg | In vivo |
| Mucizumab (Mcz) i.e. anti-sCD146 | | | 5 mg/kg | In vivo |
| antibody as described herein | | | | |
| Bispecific Mcz/Bvz antibody i.e. bispecific | | | 5 mg/kg | In vivo |
| anti-sCD146 antibody and anti-VEGF | | | | |
| comprising light and heavy chain variable | | | | |
| region from mucizumab and from | | | | |
| bevacizumab as described herein | | | | |

Legend: WB: Western Blot; IP: Immunoprecipitation; ELISA: Enzyme Linked ImmunoSorbent Assay; FC: Flow Cytometry.

Plasmids, siRNA and Cell Transfection

The pEF1-alpha-V (#27290) and pcDNA3.1-beta-3 (#27289) plasmids encoding integrin subunits αv and β3, respectively, were purchased from Addgene. The corresponding empty vectors were used as a negative control. The small interfering RNA (siRNA) targeting integrin αL (AM16708), integrin 32 (HSS105562), integrin αv (HSS105554) and integrin β3 (HSS105565) gene transcripts and the silencing negative control (AM4611) were from Invitrogen. Cell transfection was carried out using Polyplus® jetPrime reagent (Ref 114-15) according to the manufacturer's instructions. For integrin αvβ3 co-transfection experiments, equal amount of pEF1 and pcDNA3.1 vector was added.

### Western-Blot and Immunoprecipitation Assays

Briefly, cells were grown on plates and treated as a function of the experiment, then washed in PBS, scraped off the plates and extracted with 300 μl of ice-cold RIPA buffer (150 mM NaCl, 50 mM Tris HCl, pH 7.4, 2.4 mM EDTA, 1% Nonidet P40, 0.5 mM phenylmethylsulfonyl fluoride, supplemented with protease and phosphatase inhibitor (A32959)) for 30 min at 4° C. After centrifugation (12,000 g, 10 min, 4° C.) to eliminate cell debris and nuclei, the Pierce™ BCA protein assay kit was used to quantify protein concentration. 25 μg of proteins were subjected to SDS-PAGE (NuPAGE™ 4 to 12%, Invitrogen), and then transferred to a nitrocellulose membrane using iBlot2 system, Invitrogen. The membranes were blocked in TBST-5% BSA for 1 h at room temperature and then probed separately with primary antibodies overnight at 4° C. The blots were finally incubated with HRP-conjugated secondary antibody for 1 hour at room temperature and protein bands were revealed using ECL substrates (Invitrogen). Mild stripping buffer was used to strip membranes and re-probe with another primary antibody. Immunoblotting on mice brains was done on 100 mg of tissues. Briefly, mice brains were snap frozen in liquid nitrogen, minced, and three fractions of 100 mg each from the same brain was mixed with RIPA for 30 minutes on ice.

For immunoprecipitation, non-denaturing lysis buffer (20 mM Tris HCl pH 8, 137 mM NaCl, 1% Nonidet P-40 (NP-40), 2 mM MnCL2, 1 mM CaCL2) was used in all experiments unless otherwise specified. Briefly, 5 μg of primary antibody was added to cleared whole cell lysate and kept overnight on a rotator in the cold room. The next day, Dynabeads™ Protein G was added for 1 hour at 4° C. The beads were then trapped with a magnetic field, washed trice with PBS+/+ and finally resuspended in RIPA buffer. The complex was heated at 95° C. for 10 minutes and loaded on SDS-PAGE. In immunodepleting experiments, the same procedure was followed except the depleted fraction was preserved at −80° C. until use.

### ELISA Experiments

ELISA kits used to specifically detect human sCD146 and human VEGF were from BioCytex (cat #7501) and BioLegend (cat #446507), respectively. In-house ELISA was developed to detect the interaction between sCD146 and integrin αvβ3. Briefly, 0.5 μg of either recombinant sCD146 or recombinant integrin αvβ3 was added into the wells of a 96-well plate. After overnight incubation at 4° C., wells were thoroughly washed with PBS-Tween 0.05%, blocked with 4% BSA, and then the reciprocal interacting recombinant proteins were added for 1 hour at room temperature. After several washes, wells were probed with anti-integrin αvβ3 or anti-sCD146. The plate was then washed and a secondary antibody coupled to HRP was added for 30 minutes at room temperature. The interaction was revealed by adding peroxide substrate, TMB, and incubating for 5 minutes at room temperature. The reaction was finally stopped with sulfuric acid and absorbance was read at 450 nm.

### Immunofluorescence Experiments

Cells were seeded in Lab-Tek™ II Chamber Slide (Invitrogen) at 80% confluency and were then transfected with plasmids encoding or not integrin αv and β3 subunits. For siRNA experiments, cells were seeded at 50% confluency and transfected after 24 hours with integrin β3 siRNA. 48 hours post-transfection, cells were washed twice with PBS, serum-starved for 2 hours, and recombinant sCD146-FITC protein was then added for 30 minutes at 37° C. An irrelevant IgG-FITC was used as a negative control. The cells were then washed twice with PBS, fixed with 4% PFA, and nuclei were counterstained with DAPI (BioLegend). The slides were finally mounted in ProLong Glass antifade (Invitrogen). Images were visualized using Leica inverted microscope (Leica DFC9000 GTC).

### Flow Cytometry Experiments

Flow cytometry experiments were conducted with a Gallios Flow cytometer (Beckman Coulter, CA, USA). Briefly, all detached cells were labeled with the antibody or isotype matched control antibody directly coupled to fluorescent dye (10 μg/ml) for 30 min at 4° C. After washing, samples were analyzed by flow cytometry. Dead cells were omitted from analysis using LIVE/DEAD™ Fixable Aqua dye (Invitrogen). Isotype controls were used to exclude false positive cells. Results were analyzed with Kaluza software (Beckman Coulter, CA, USA).

### Reverse Transcription-Quantitative PCR (RT-qPCR)

mRNA expression of several genes was studied by reverse transcription (RT) and quantitative polymerase chain reaction (qPCR). Cells were incubated in experimental conditions. Total RNA was extracted using the ReliaPrep™ RNA Cell Miniprep System (Promega), according to the manufacturer's instructions. DNA was digested with DNase (RNase-Free DNase Set, Qiagen) to secure complete DNA removal. Reverse transcription using random primers was performed on 1 μg of total RNA of each sample using Superscript II reverse transcriptase (Invitrogen). 50 ng of cDNA were then subjected to RT-qPCR using pre-designed TaqMan primers (Invitrogen). The housekeeping gene GAPDH was used as an internal control to normalize gene expression. The relative expression level of a particular gene was evaluated using the 2-ΔΔCt method. Each point was run in triplicate.

### Cell Migration Assays

Cells were seeded in 24-well plates at 90% confluency. The following day, once at confluency, cells were serum-starved for 4 hours and then a hole was made in the center of each well. A reproducible hole was performed using a 200 μl pipet tip. After 3 washes with minimal medium, cells were stimulated with either rsCD146, VEGF, or conditioned media depleted or not from these factors. The first image acquired was assumed to be at time T0. Images were then automatically taken every 2 hours for up to 8 hours in environmentally controlled conditions (37° C., 5% CO2) on Leica microscope. The extent of cell migration was calculated according to the following formula: 100−((A1/A0)*100) whereby A0 is the area of the hole at time T0 and A1 is that after 8 hours of migration as calculated using ImageJ software.

### Cell Proliferation Assays

5×10³ U87, U118 or U373 cells were seeded in 96-well plates for 24 hours. Cells were serum-starved for 4 hours and then exposed to different treatments as described in the legend of FIG. 3. Cell proliferation was assayed by WST-1 proliferation kit (Sigma-Aldrich) after 48 hours of stimulation. WST-1 cell proliferation assay utilizes atetrazolim salt WST-1[2-(4-Iodophenyl)-3-(4-nitrophenyl)-5-(2,4-disulfophenyl)-2H-tetrazolium]. WST-1 produces a highly water-soluble formazan upon metabolically active cells, allowing a direct and user-friendly colorimetric measurement of cell proliferation. Briefly, 10 μL of WST-1 reagent was added into the medium and incubated for 2 hours at 37° C. before measuring the absorbance value at 450 nm using the microplate reader GloMax (Promega).

### Transwell Invasion Assays

Invasion assay were conducted to examine the invasive capacity of the cells. Briefly, cells were stimulated for 48 hours with different treatments as described in the figure legend and then 25,000 were seeded in upper transwell chambers (8 μm pore size; Corning) previously coated with serum-reduced Matrigel (Corning). Complete media containing 10% FCS was added to the lower chamber. After 48 hours, non-invaded cells were scrapped off while cells on the other face of the insert were fixed with 4% paraformaldehyde and stained with 0.5% crystal violet. The number of cells from three separate fields in each well was counted. Each assay was performed in triplicate and representing images are shown.

### Crispr/Cas9 Deletion of Genes

The vector pSpCas9(BB)-2A-Puro (PX459) V2.0, expressing both Cas9 and sgRNA scaffold was purchased from Addgene. gRNAs targeting the genes CD146-Exon 6, ITGB3-Exon 1, and VEGFR2-Exon 1 were designed using CRISPOR tool (http://crispor.org/) and selected based on minimal off-target score. The 5′-3′ oligo sequences are as follow; CD146-Exon 6 sense: CACCGTCAACTACCGGCTGCCCAGT (SEQ ID NO: 44), CD146-Exon 6 anti-sense: AAACACTGGGCAGCCGGTAGTTGAC (SEQ ID NO: 45), ITGB3 Exon 1 sense: CACCGAGGCGGACGAGATGCGAGCG (SEQ ID NO: 46), ITGB3 Exon 1 anti-sense: AAACCGCTCGCATCTCGTCCGCCTC (SEQ ID NO: 47), VEGFR2 Exon 1 sense: CACCGTGCTGCTGGCCGTCGCCCTG (SEQ ID NO: 48), VEGFR2 Exon 1 anti-sense: AAACCAGGGCGACGGCCAGCAGCAC (SEQ ID NO: 49). The PX459 plasmid was digested with BbsI and gel purified using the Wizard SV Gel and PCR Clean-Up System (Promega). A pair of oligos for each target site was phosphorylated, annealed and ligated into linearized pX459 vector for generating gRNA-expressing plasmid. The resulting plasmids were separately transformed into JM109 competent cells and minipreps were carried out on the resulting colonies. U87 and U373 cells were then transfected with 10 μg of gRNA-containing plasmids using jetPrime (PolyPlus) according to the manufacturer's protocol. Cells were cultured for 10-14 days with puromycin (2 μg/mL) and single cell-derived colonies were finally harvested and amplified. Genomic DNA from the CRISPR-Cas9 modified cells was isolated and sequences encompassing sgRNA-targeted regions were PCR-amplified (Platinum Taq DNA Polymerase, Invitrogen) using primers flanking the targeted sequence of the sgRNAs. Amplified fragments were then sequenced (Illumina HiSeq 2500).

### Experiments on Animals and Imaging

Four to five weeks old NMRI-Foxn1nu female mice (Janvier-labs) were used. For anesthesia, either isoflurane gas or ketamine/xylazine IP injection was used.

In U87 ectopic tumor model, xenograft of the human glioblastoma cell line U87 was produced by subcutaneously injecting 10⁶ cells into the back ofNMRI-foxn1 nude mice. When tumors reached 20 mm³, peri-tumoral administration of irrelevant human IgG1, humanized anti-sCD146 “mucizumab”, anti-VEGF “bevacizumab”, a combination of both humanized anti-sCD146 “mucizumab” and anti-VEGF “bevacizumab” antibodies, or of a bispecific antibody comprising the light and heavy chain variable region of bevacizumab and mucizumab (cf. table 1) began at a dose of 10 μg, twice a week for 4 weeks. Tumor size was measured once a week with a caliper and tumor volume was determined according to the equation: ((length*width*height)*7/6).

In U87 orthotopic tumor model, orthotopic injections of U87 cells (10⁵ in 3 μL PBS) were performed using a stereotactic frame (Stoelting) at 2 mm on the right of the medial suture, 1 mm in front of the bregma, and at a depth of 2.5 mm. Cells were injected on the same day into 4 weeks old NMRI-Foxn1nu female mice. Ten days post-implantation, mice were randomized and regrouped into i) control IgG, ii) bevacizumab, iii) mucizumab, iv) mucizumab+bevacizumab and bispecific Mcz/Bvz receiving groups. Antibodies (5 mg/kg) were retro-orbitally administered and thereafter every two days for up to two weeks. Animals were then anesthetized, perfused with PBS followed by 4% PFA, and brains were finally isolated. At least four mice were used in each group. The brains were then fixed in PFA for 24 hours, saturated in increasing concentrations of sucrose, and stored in isopentane at −80° C. until use.

In representative animals of the U87 orthotopic tumor model, MicroPET/CT acquisitions were performed on a NanoscanPET/CT camera (Mediso, Budapest, Hungary) using 86Ga-RGD. Radioactivity was injected intravenously in the retro-orbital sinus. Mice were maintained under 2% isoflurane anesthesia during acquisition. Static microPET imaging was performed 1 h after each radiotracer injection, during 20 min.

### Immunohistochemistry

At the end of the experiments, animals were perfused at room temperature with PBS then with paraformaldehyde (PFA) 4% at room temperature before sacrifice. Brains were then removed and incubated for 24 h at 4° C. in PFA 4%, then for 8 h at 4° C. in sucrose 20%, and finally for 3 days at 4° C. in sucrose 30%. Brains were then stored at −80° C. in isopentane. Cryopreserved brain tissues were serially cut into 10 μm-thick coronal sections using a Cryostat (Leica, CM 3050S, Wetzlar, Germany) at −20° C. For immunohistochemistry, brain sections were brought out of −80° C. and allowed to reach room temperature. Slides were then rehydrated in a series of TBS bath and blocked in TBS-2% BSA for 1 hour at room temperature. The brain sections were then incubated with HRP-conjugated primary antibody directed against human CD146 (clone COM7A4-HRP, 1:20) overnight at 4° C. Staining was visualized by using 3, 30-diaminobenzidine (DAB), followed by hematoxylin counterstaining (Sigma-Aldrich). Slides were scanned using Leica microscope (Leica DFC9000 GTC). Images panels were prepared using Fiji or ImageJ software. To estimate tumor volume, Immunohistochemistry Image Analysis Toolbox (Shu J. et al., 2014) was used in ImageJ software. The tumor area in each section was calculated, and then the volume was estimated by multiplying with tumor thickness.

### Patient Cohort

Inventors retrospectively included all adult patients referred to their institution for recurrent Isocitrate dehydrogenase (IDH) wild-type glioblastoma who received bevacizumab at the dose of 15 mg/kg every 3 weeks in association with carmustine at the dose of 150 mg/m² every 6 weeks for whom plasma samples were available before bevacizumab administration (D1) and after 21 days from the first bevacizumab administration (D21). Clinical and imaging evaluations were performed every 3 weeks and 6 weeks respectively. Treatment responses and disease progression were reviewed using the RANO criteria (Wen Py et al., 2010). All patients provided written informed consent in accordance with institutional, national guidelines and the Declaration of Helsinki.

Plasma samples were collected at baseline, before bevacizumab administration (D1), and after the first bevacizumab administration (D21).

### Statistical Analysis

The data are expressed as mean values±SEM.

Categorical variables were presented as frequencies and percentages, continuous variables as median and range. Overall survival (OS) was defined to be the time from first bevacizumab administration to death from any cause, censored at the date of last contact. Progression-Free Survival (PFS) was the time from first bevacizumab administration to documented progression or death, censored at the date of the last documented disease evaluation. Kaplan-Meier method was used to estimate survival curves. Log-rank tests were used for univariate comparisons. sCD146 plasmatic levels were dichotomized by their median values for survival analyses. Mann-Whitney U-test or Kruskal Wallis test were used to compare quantitative and qualitative values; qualitative values were analyzed by Fisher exact test and Chi 2 test.

Statistical analysis was performed using Prism software (GraphPad Software Inc., San Diego, CA, USA) or SPSS® v22. The variance between the different groups to be compared was estimated before statistical analysis. Significant differences between two groups were determined using the unpaired, 2-tailed, Mann-Whitney U test. A value of P≤0.05 was considered to be significant.

### Results

Soluble CD146 is Increased in the Plasma of Glioblastoma Patients and its Increase is Associated with Poor Progression-Free Survival (PFS) and Overall Survival (OS) after Bevacizumab Treatment

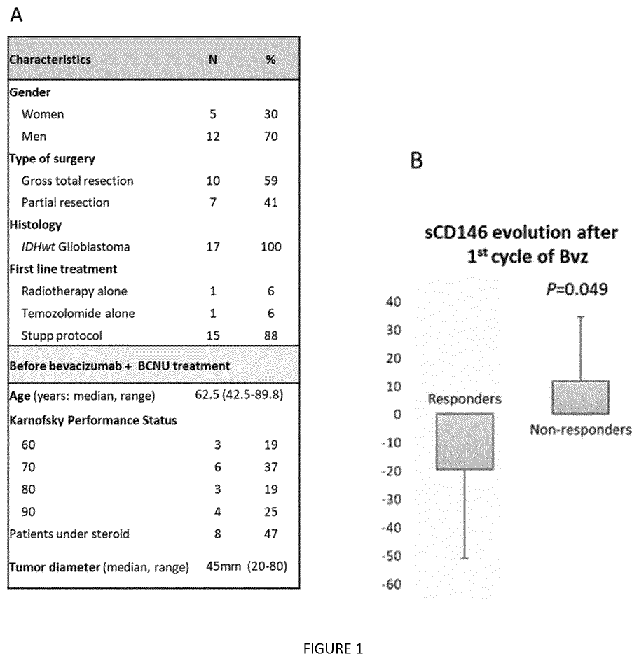

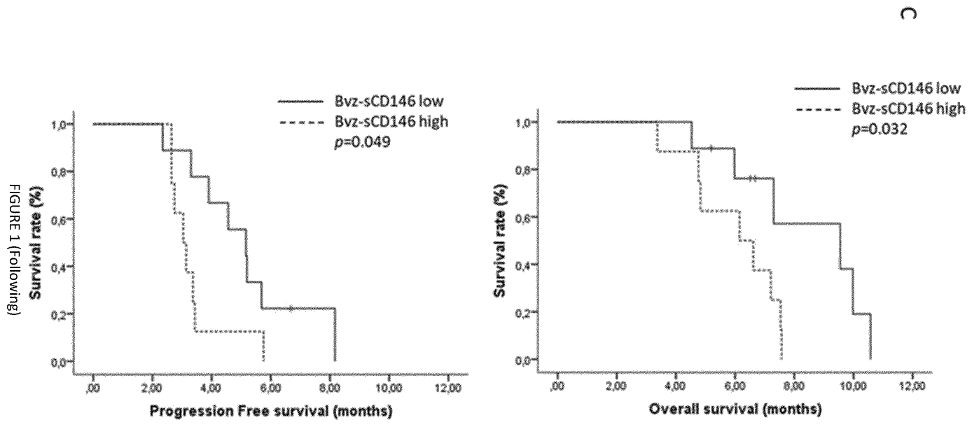

Inventors measured the plasma concentration of soluble CD146 (sCD146) in a cohort of 17 patients with recurrent IDHwt glioblastoma who were treated for 3 weeks with bevacizumab and carmustine (FIG. 1A). Plasma samples were collected just before the second bevacizumab administration. Responses after bevacizumab-chemotherapy administration were either complete (1 patient) or partial (5 patients), corresponding to responders, or stable (4 patients) or progressive (7 patients), corresponding to non-responders. Inventors show that, under bevacizumab treatment (after one cycle of treatment), sCD146 plasma level increases more in non-responder patients than in responders (FIG. 1B). Importantly, a high sCD146 plasma level in patients treated with one cycle of bevacizumab significantly associated with shorter PFS (p=0.049) and OS (p=0.032) (FIG. 1C). Patients with a high sCD146 plasma concentration had a median PFS of 3.0 months (95% CI: 2.5-3.6) as compared to 5.2 months (95% CI: 3.4-6.9) in patients with low sCD146 plasma concentration. Patients with a high sCD146 plasma concentration had a median OS of 6.1 months (95% CI: 3.7-8.6) versus 9.6 months (95% CI: 4.8-14.3) in patients with low sCD146 plasma concentration. In contrast, neither age, Karnofsky score (KPS) nor steroid dose were associated with patient PFS or OS in univariate analyses, suggesting the independence of sCD146 from classical prognostic factors.

These results highlight the pejorative impact of sCD146 increase in patients treated with bevacizumab and its implication in bevacizumab resistance.

Long-Term Treatment with Bevacizumab Significantly Induces CD146/Soluble CD146 Signaling Pathway in CD146-Positive Glioblastoma Cell Lines

In this study, inventors used 3 different glioblastoma cells lines: U87, U373 and U118. Flow cytometry analysis of U87 and U373 glioblastoma cell lines demonstrated potent expression of CD146, VEGFR2 and integrin αvβ3 in these two cell lines. In contrast, U118 cells did not express integrin αvβ3 and VEGFR2, only faintly expressed CD146, and were thus used as a negative control. The secretion capacity of sCD146 and VEGF from the three glioblastoma cell lines was determined in vitro. ELISA experiments on culture supernatant evidenced a high secretion of sCD146 in U87 and U373 cell lines while U118 cells did not secrete sCD146. In contrast, U87, U373 and U118 highly secreted the Vascular Endothelial Growth Factor (VEGF) (FIG. 6).

To generate Bevacizumab-resistant GBM cell lines, inventors treated U87, U373 and U118 for two months with 100 μg/ml bevacizumab (commercially known as Avastin) or irrelevant IgG as a control. The absence of antibody toxicity was confirmed using trypan blue exclusion assay and live/dead fixable aqua staining dye. After 2 weeks of treatment, they noticed a robust increase in U87 and U373 cell proliferation, but not in U118 cell proliferation, an effect that was maintained during the entire treatment course (FIG. 2A; FIG. 7A; FIG. 8A). In addition, a significant increase in CD146, VEGFR2, integrin subunits αv and β3 at the mRNA levels was detected in U87 and U373 cell lines, but not in U118 cells (FIG. 2C; FIG. 7C; FIG. 8C). As membrane CD146 expressed at the membrane can be shed to generate sCD146, inventors analyzed the membrane expression of these proteins by flow cytometry. The membrane expression of integrin αvβ3 and VEGFR2 were increased but CD146 membrane expression decreased (FIG. 2D; FIG. 7D). In contrast, membrane expression of the three surface proteins did not change in U118 cells (FIG. 8D).

Besides, sCD146 concentration was significantly increased in the culture media of U87 and U373 cells after exposure to bevacizumab as compared to irrelevant IgG (FIG. 2B; FIG. 7B). This increase in sCD146 concentration was not detected in the supernatant of bevacizumab-treated U118 cells (FIG. 8B).

Inventors then examined Epithelial-Mesenchymal Transition (EMT) and Cancer Stem Cells (CSC) markers in the bevacizumab-challenged glioblastoma cells. They analyzed the main CSC markers oct4, sox2 and nanog, and the EMT markers that are commonly expressed in GBM cells, snail, slug, vimentin, N-cadherin, S100A4, Twist1, and Zeb1, at the mRNA level. All these markers were potently upregulated in the bevacizumab challenged U87 cell line as compared to IgG treated cells (FIG. 2 E-F). Similar results were obtained in the U373 cell line (FIG. 7 E-F) but not in the CD146-negative glioblastoma cell line U118 (FIG. 8 E-F).

sCD146 Reproduces the Effects Observed with Long-Term Bevacizumab Treatment in CD146-Positive Glioblastoma Cells

As bevacizumab treatment of CD146-positive (CD146+) glioblastoma cells led to a potent increase in sCD146 secretion, inventors investigated sCD146 effect on U87 and U373 cells. Their results show that in vitro stimulation of these cells for 48 hours with recombinant sCD146 or VEGF significantly increased cell proliferation, migration, and invasion. An additive outcome effect was achieved when combining both molecules, in comparison to VEGF or sCD146 treatment alone (FIG. 3 A-C; FIG. 9 A-C). In contrast, in U118 cells, sCD146 or VEGF failed to induce cell proliferation (FIG. 10).

Moreover, sCD146 potently induced the expression of the mesenchymal markers snail, slug, N-cadherin and vimentin, but decreased the epithelial marker E-cadherin at the protein level in U87 cells. Likewise, sCD146 upregulated the expression of the cancer stem cell markers nanog, oct4 and sox2 in U87 cells (FIG. 3 D-E). Similar results were obtained in U373 cell lines (FIG. 9 D-E).

Predictive Value of Basal sCD146 Concentration on Response of Patients to Bevacizumab

Inventors analyzed the influence of plasma sCD146 concentration before bevacizumab treatment (basal sCD146) in 17 patients with glioblastoma (GBM) on their response to bevacizumab. Results showed that basal sCD146 was significantly higher in non-responder patients, as compared to responder patients. These results shows that basal sCD146 constitute a very early and non-invasive marker of response to bevacizumab in GBM patients (cf. FIG. 22).

sCD146 Mediates its Effects Through Integrin αvβ3 in CD146-Positive Glioblastoma Cells

In a previous work, inventors showed that sCD146 interacts with angiomotin on endothelial cells to promote angiogenesis (Stalin et al., 2013). Thus, they examined angiomotin expression on U87, U373, and U118 cells. Flow cytometry analysis showed a weak expression of this surface protein. In contrast, integrin subunits αv and β3 were expressed by U87 and U373 but not U118 cells as assessed by RT-PCR and flow cytometry. Therefore, inventors tested a possible interaction between sCD146 and integrin αvβ3. Silencing RNA targeting integrin subunits αv or β3 significantly reduced sCD146-induced increase in proliferation of U87 and U373 cells, whereas siRNA targeting other integrin subunits, αL or β2, did not modify cell proliferation. Moreover, knocking down integrin αvβ3 using silencing RNA targeting either αv or β3 subunits, but not αL or β2 integrins, on U87 and U373 cells, led to a significant decrease in sCD146-FITC binding to these cells. In order to confirm the specific binding of sCD146 on αvβ3, they transfected Chinese Hamster Ovary cells (CHO), which originally do not express this integrin, with αvβ3. Exogenous expression of integrin subunits αv and β3 into CHO cells induced a significant increase in sCD146-FITC binding to these cells as revealed by flow cytometry and immunofluorescence experiments. In addition, anti-αvβ3 antibody immunoprecipitated sCD146 only when CHO cells expressed this integrin after sCD146 treatment. To confirm the specificity of the immunoprecipitated sCD146, inventors treated CHO cells with blocking anti-αvβ3 antibody before adding sCD146. Results show that anti-αvβ3 antibody potently reduced the interaction with sCD146.

In-house ELISA also confirmed a strong interaction between integrin αvβ3 and sCD146 with an estimated dissociation constant of 1.8 nM. The addition of blocking cyclic RGD peptide failed to inhibit this interaction even at high concentrations, in contrast to a newly generated humanized neutralizing monoclonal anti-sCD146 antibody, mucizumab, which was generated by inventors' laboratory.

Finally, inventors overexpressed or silenced integrin αvβ3 in U118 and U87 cells and studied EMT markers in response to sCD146 stimulation. Results showed that only cells expressing αvβ3 upregulate EMT markers when treated with sCD146.

CD146 is Part of a Signalosome Containing VEGFR2 and Integrin αvβ3

To analyze the components of the signalosome containing CD146, inventors performed co-immunoprecipitation experiments using anti-CD146 antibody, S-Endo1. Immunoblotting with anti-VEGFR2, anti-αv, and anti-β3 antibodies revealed specific bands corresponding to these proteins. Membrane probing with an antibody to another surface protein, EPCAM, failed to detect it, attesting the specificity of the immunoprecipitated proteins (FIG. 11 A). Results were reproduced in U373 cell line (FIG. 12 A).

As CD146 is a co-receptor for VEGFR2, and since VEGFR2 activation induces its own phosphorylation at multiple tyrosine residues (Wang D et al., 2020), inventors investigated whether sCD146 or VEGF induces the phosphorylation of CD146 and whether integrin αvβ3 is indispensable in this process. Accordingly, U87 cells were treated with either sCD146 or VEGF, and CD146 was immunoprecipitated followed by western blotting using anti-pan phospho-tyrosine antibody. Results showed that sCD146 and VEGF increased the phosphorylation of membrane CD146 (FIG. 11 B). Next, they knocked-down integrin αvβ3 using silencing RNA and reproduced the experiment. A potent decrease in CD146 phosphorylation in response to sCD146 or VEGF stimulation was detected as compared to control cells transfected with scrambled RNA (FIG. 11 B). These results were reproduced in U373 cells (FIG. 12 B).

Inventors then studied the phosphorylation of integrin β3. Their results show a significant increase in integrin β3 phosphorylation in response to sCD146 or VEGF stimulation (FIG. 11 C). To examine if CD146, integrin αvβ3, or VEGFR2 are crucial in the phosphorylation and activation of the newly defined signalosome, they used CRISPR-Cas9 technology to individually knockout each of these proteins. Inventors showed that the knockout of CD146 on U87 cells significantly reduced integrin β3 and VEGFR2 phosphorylation following sCD146 or VEGF stimulation. Also, knocking out integrin β3 on U87 cells significantly reduced or abolished VEGFR2 phosphorylation in response to VEGF or sCD146 stimulation (FIG. 11 D). In contrast, VEGFR2 knockout on U87 cells abrogated integrin β3 phosphorylation in response to VEGF stimulation but not sCD146 (FIG. 11 C). Similar results were obtained in U373 cells (FIG. 12 C-D).

Of interest, Inventors also showed this signalosome to exist in human umbilical endothelial cells, HUVEC (FIG. 13A), and revealed that sCD146, through integrin αvβ3, induces cell proliferation (FIG. 13B) and migration (FIG. 13C).

These results demonstrate a complex constituted by the surface proteins CD146, VEGFR2 and integrin αvβ3 that mediates sCD146 and VEGF effects on CD146+ glioblastoma cells, but also on endothelial cells.

sCD146 and VEGF Activate Common Signaling Pathways in CD146-Positive Glioblastoma Cell Lines

Inventors next investigated the signaling pathways that are implicated in mediating sCD146 and VEGF effects in CD146+ glioblastoma cells. To this end, they studied the phosphorylation of key proteins involved in major signaling pathways regulating cell survival, proliferation, and invasion. Inventors' results show that, similar to VEGF, sCD146 induced the phosphorylation of P38 MAPK, AKT, and ERK44/42 proteins. Besides, both sCD146 and VEGF induced the phosphorylation and activation of focal adhesion kinase (FAK). The phosphorylation of these proteins became significantly impaired when either CD146, VEGFR2, or integrin αvβ3 were knocked out. These effects were functionally translated to a decrease in the ability of cells to proliferate and migrate in response to sCD146 and/or VEGF stimulation (FIG. 14; FIG. 15). Thus, CD146, VEGFR2 and integrin αvβ3 interact together to translate extracellular stimuli into intracellular signals.

A Novel Humanized Anti-sCD146 Antibody, Mucizumab, Blocks sCD146 Adverse Effects on CD146-Positive Glioblastoma Cells and Prevents Escape from Bevacizumab In Vitro

In order to block glioblastoma escape from bevacizumab, Inventors tested the effect of a new humanized neutralizing monoclonal anti-sCD146 antibody they've developed, mucizumab. The addition of 100 μg/ml of either bevacizumab, mucizumab, or the combination of both antibodies to U87 or U373 conditioned media (CM) resulted in a significant decrease in cell proliferation, migration, and invasion in vitro. Inventors' results showed that mucizumab displayed greater effects than that achieved by bevacizumab, while the combination of mucizumab and bevacizumab significantly extended the inhibitory effects of bevacizumab on cells' proliferation, migration, and invasion as compared to bevacizumab treatment alone (FIG. 4 A-C; FIG. 16 A-C).

Inventors then reproduced the long-term bevacizumab challenging experiment and added mucizumab to the media along with bevacizumab. The effect on EMT and CSC induction in U87 and U373 cells was evaluated. Inventor's results showed a robust decrease in the tested EMT and CSC markers when mucizumab was added to bevacizumab (FIG. 4 D-G; FIG. 16 D-E).

These data confirm that CD146+ glioblastoma cells escape bevacizumab through sCD146 secretion.

Combination of Bevacizumab with Mucizumab Exhibits Greater Inhibitory Effects than Bevacizumab Alone on Tumor Growth in Different Preclinical Models

To investigate the in vivo relevance of combining bevacizumab with mucizumab, U87 cells were xenografted subcutaneously in athymic nude mice and tumor growth was weekly monitored with caliper. After 5 weeks of treatment, results showed that mice receiving mucizumab developed significantly smaller tumors than those receiving IgG. Of importance, a synergic inhibitory effect on tumor growth was detected when bevacizumab was combined with mucizumab (FIG. 5 A). Inventors measured the concentrations of human VEGF (hVEGF) and human sCD146 (hsCD146) in the plasma of treated mice. Inventors' results showed that the combination of bevacizumab and mucizumab significantly decreased hVEGF and hsCD146 concentrations in the plasma from treated mice as compared to IgG-control group whereas bevacizumab alone increased hsCD146 (FIG. 17 A-B).

Inventors also performed another in vivo study by orthotopically injecting U87 cells in nude mice. To evaluate the therapeutic benefit of the administered antibodies, immunohistochemistry was done to estimate tumor growth. Similar to that in ectopic tumor model, mucizumab significantly reduced tumor volume and metastasis, while the combination of mucizumab and bevacizumab greatly enhanced these effects. In contrast, bevacizumab, as a monotherapy, did not decrease intracranial tumor growth but rather increased tumor dissemination across the brain parenchyma (FIG. 5 B-C). In addition, immunoblots using brain lysate from treated mice showed that the human proteins CD146 and MAX (a DNA-binding transcriptional regulator used as a human protein internal control) decreased when treating with bevacizumab and mucizumab to reach barely detectable levels when both antibodies were combined as compared to IgG-treated group (FIG. 5 D). This indicates a large decrease in human tissue corresponding to GBM after treatment with both antibodies. This was confirmed by PET imaging showing a slight significant decrease of the tumor when bevacizumab was used, whereas a large decrease was observed in the presence of both bevacizumab and mucizumab. Along this line, the decrease was significantly higher in the bevacizumab+mucizumab group, as compared to bevacizumab group (FIG. 5 E). Also, inventors showed a significant decrease in plasma concentration of hVEGF and hsCD146 in mice receiving simultaneously bevacizumab and mucizumab antibodies as compared to IgG-control group (FIG. 17 C-D).

These results emphasize the interest of combining anti-VEGF and anti-sCD146 therapy for increasing significantly inhibitory effects on glioblastoma growth.

Bispecific Antibody Resulting from the Fusion of Mucizumab Exhibits Greater Inhibitory Effects than Bevacizumab Alone or Mucizumab Alone on Tumor Growth in Different Preclinical Models.

Inventors generated a bispecific antibody comprising the light and heavy chain variable regions from bevacizumab and mucizumab. They tested the effect of bevacizumab (Bvz), mucizumab (Mcz), bevacizumab+mucizumab (Bvz+mcz) and bispecific anti-VEGF/sCD146 (Bispecific) antibodies, as compared to control IgG, in an orthotopic model of U87 glioblastoma cells injected in nude mice. After 2 weeks of treatment, mice were sacrificed and brain weight was determined. Results show that all antibodies have a significant effect on the brain weight, as compared to the IgG control group. Of interest, both the combination of Bvz and Mcz, and the bispecific antibody significantly reduced brain weight, as compared to Bvz alone and Mcz alone (FIG. 18). As tumor is part of the brain, it can be assumed that the variations observed on brain weight correspond directly to variations of tumor weight.

The effects of bevacizumab, mucuzumab, bevacizumab+mucizumab and the bispecific anti-VEGF/sCD146 antibody was also measured in an orthotopic model of human glioblastoma in mice by PET imaging. After 2 weeks of treatment, mice were used for PET imaging using the ⁸⁶Ga-RGD tracer. Results show that all antibodies have a significant effect on the tumor size, as compared to the IgG control group. Of interest, both the combination of Bvz and Mcz, or the bispecific antibody significantly reduced tumor size as compared to Bvz alone (FIG. 19).

Differential Effects of Bevacizumab, Mucizumab, Bevacizumab+Mucizumab and the Bispecific Anti-VEGF/sCD146 Antibody on Human CD146 mRNA Expression in an Orthotopic Model of Human Glioblastoma in Mice.

Inventors also determined the human CD146 mRNA expression after 2 weeks of treatment. Mice were sacrificed and brains were dissected. Results show that Bvz+Mcz and bispecific antibody have a significant effect on the human CD146 mRNA expression, as compared to the IgG control group. Both the combination of Bvz and Mcz, and the bispecific antibody significantly reduced human CD146 mRNA expression, as compared to Bvz alone or Mcz alone. Of interest, bispecific effect was significantly higher than the Bvz+Mcz effect on human CD146 mRNA expression (FIG. 20). In these experiments, it can be assumed that human CD146 mRNA expression is correlated with the number of human cells constituting the tumor and thus with the tumor size. Inventors conclude that the combination herein described for the first time advantageously and significantly increases inhibitory effects on glioblastoma growth, the bispecific antibody optimizing said inhibitory effects.

Differential Effects of Bevacizumab, Mucizumab, Bevacizumab+Mucizumab and the Bispecific Anti-VEGF/sCD146 Antibody on Human sCD146 and VEGF Concentrations in an Orthotopic Model of Human Glioblastoma in Mice.

Finally, inventors measured the effect of each antibody on the human sCD146 and VEGF concentrations. After 2 weeks of treatment, mice were sacrificed and blood concentrations of VEGF and sCD146 were determined by ELISA. Results show that all antibodies have a significant effect on human VEGF concentration, as compared to the IgG control group. Likewise, all antibodies have a significant effect on human sCD146 concentration, except Bvz. In addition, Bvz+Mcz and bispecific antibody have a significant effect on the human sCD146 and VEGF concentrations, as compared to Bvz alone (FIG. 21). These results show the effect of the different antibodies on the concentrations of circulating sCD146 and VEGF.

### Discussion

In this study inventors showed that, in patients with glioblastoma treated with bevacizumab, an increase in soluble CD146 (sCD146), an easily quantifiable circulating biomarker, as soon as at the end of the first cycle of treatment is associated with a decrease in PFS and OS. They also demonstrated the predictive value of basal (i.e. before treatment) sCD146 concentration on response of patients to bevacizumab. Thus, sCD146 is an early marker of resistance to this therapy. To confirm this hypothesis, inventors deciphered the sCD146-mediated molecular mechanisms leading to bevacizumab escape. They show in vitro that bevacizumab-resistant CD146+ glioblastoma cells are able to potently increase CD146 expression and enhance sCD146 secretion. The secreted sCD146 is able to exert autocrine effects on the glioblastoma cells to stimulate their proliferation, migration and invasion. In the same way, sCD146 robustly increases cellular markers of epithelial to mesenchymal transition and cancer stem cell markers in CD146+glioblastoma cells. These autocrine effects are mediated through the interaction of sCD146 with integrin αvβ3 which is overexpressed by glioblastoma cells after bevacizumab treatment.

To block sCD146 adverse effects, inventors generated a novel humanized monoclonal anti-sCD146 antibody, named mucizumab, that specifically targets and neutralizes the soluble form of CD146 (sCD146) while maintaining minimal reactivity to membrane CD146 which displays physiological functions. Of importance, the combination of bevacizumab and mucizumab antibodies exhibits a therapeutic benefit by significantly decreasing Epithelial-Mesenchymal Transition (EMT) and Cancer Stem Cells (CSC) in CD146+ bevacizumab-resistant glioblastoma cells in vitro, as well as potently decreasing tumor growth in a pre-clinical model of glioblastoma orthotopically injected in nude mice. Advantageously, the use of a bispecific antibody combining the variable regions of mucizumab and bevacizumab dramatically decreases tumor growth in the same animal model more efficiently than mucizumab alone or bevacizumab alone.

### CONCLUSION

This study identifies sCD146 as 1/an easily detectable and early non-invasive biomarker to predict and monitor the response to a bevacizumab treatment of a subject suffering from glioblastoma and 2/a target to prevent or overcome bevacizumab resistance in patients with glioblastoma. This leads the inventors to the herein described invention directed to a targeted therapy combining bevacizumab and mucizumab, either by combining these two antibodies in a composition or, more advantageously, by using anti-sCD146/anti-VEGF bispecific antibody, enhancing therapeutic benefits in patients with glioblastoma, or in other words in patients suffering of glioblastoma who have received a bevacizumab treatment (such a treatment leading to the expression of CD146 by treated glioblastoma cells).

### REFERENCES

Bonekamp D, Mouridsen K, Radbruch A, et al. Assessment of tumor oxygenation and its impact on treatment response in bevacizumab-treated recurrent glioblastoma. J Cereb Blood Flow Metab. 2017.37:485-494
Brinkmann and Kontermann, MAbs. 2017 February-March; 9(2): 182-212
Chari et al., Cancer Res. 52: 127-131 (1992)
Charlton, Methods in Molecular Biology, Vol. 248 (B. K. C. Lo, ed., Humana Press, Totowa, NJ, 2003
Crane C A, Ahn B J, Han S J, Parsa A T. Soluble factors secreted by glioblastoma cell lines facilitate recruitment, survival, and expansion of regulatory T cells: implications for immunotherapy. Neuro Oncol. 2012. 14:584-595
DespoixNb et al., Muse CD146/MCAM is a marker of natural killer cell maturation; Molecular Immunology, 2008
Gerngross, Nat. Biotech. 22: 1409-1414 (2004)
Graham et al., J. Gen Virol. 36:59 (1977)
Joshkon A, Heim X, Dubrou C, et al. Role of CD146 (MCAM) in Physiological and Pathological Angiogenesis—Contribution of New Antibodies for Therapy. Biomedicines. 2020. 8:633
Labrijn et al., 2014 Nat Protoc. 9(10):2450-63
Li et al., Nat. Biotech. 24:210-215 (2006)
Lu K V, Bergers G. Mechanisms of evasive resistance to anti-VEGF therapy in glioblastoma.
CNS Oncol. 2013. 2:49-65
Mather, Biol. Reprod. 23:243-251 (1980)
Mather et al., Annals N. Y. Acad. Sci. 383:44-68 (1982)
Osuka S, Van Meir E G Overcoming therapeutic resistance in glioblastoma: the way forward. J Clin Invest. 2017. 127:415-426
Remington: The Science and Practice of Pharmacy 20th edition (2000)
Ridgway et al. Protein Eng, 9 (1996), pp. 617-621
Sambrook J, Russell D (2001) Molecular cloning: a laboratory manual, Third Edition Cold Spring Harbor
Schaefer et al. Proc Natl Acad Sci USA 2011, 108:11187-11192
Shu J, Qiu G, Ilyas M. Immunohistochemistry (IHC) Image Analysis Toolbox. 2014. 28
Singh A, Settleman J. EMT, cancer stem cells and drug resistance: an emerging axis of evil in the war on cancer. Oncogene. 2010. 29:4741-4751
Stalin J, Harhouri K, Hubert L, et al. Soluble Melanoma Cell Adhesion Molecule (sMCAM/sCD146) Promotes Angiogenic Effects on Endothelial Progenitor Cells through Angiomotin. J Biol Chem. 2013. 288:8991-9000
Stalin J, Nollet M, Garigue P, et al. Targeting soluble CD146 with a neutralizing antibody inhibits vascularization, growth and survival of CD146-positive tumors. Oncogene. 2016. 35:5489-5500
Stalin J, Traboulsi W, Vivancos-Stalin L, et al. Therapeutic targeting of soluble CD146/MCAM with the M2J-1 monoclonal antibody prevents metastasis development and procoagulant activity in CD146-positive invasive tumors. International Journal of Cancer. 2020. 147:1666-1679
Urlaub et al., Proc. Natl. Acad. Sci. USA 77:4216 (1980)
Wang S, Chen K, Lei Q, Ma P, Yuan A Q, Zhao Y, Jiang Y, Fang H, Xing S, Fang Y, Jiang N, Miao H, Zhang M, Sun S, Yu Z, Tao W, Zhu Q, Nie Y, Li N. The state of the art of bispecific antibodies for treating human malignancies. EMBO Mol Med. 2021 Sep. 7; 13(9):e14291.
Wang D, Duan H, Feng J, et al. Soluble CD146, a cerebrospinal fluid marker for neuroinflammation, promotes blood-brain barrier dysfunction. Theranostics. 2020. 10:231-246
Weller M, Wick W, Aldape K, et al. Glioma. Nat Rev Dis Primers. 2015. 1:1-18.
Wen P Y, Macdonald D R, Reardon D A, et al. Updated response assessment criteria for high-grade gliomas: response assessment in neuro-oncology working group. J Clin Oncol. 2010. 28:1963-1972
Yazaki and Wu, Methods in Molecular Biology, Vol. 248 (B. K. C. Lo, ed., Humana Press, Totowa, NJ), pp. 255-268 (2003)

1-15. (canceled)

16. An antibody comprising an antigen binding region that specifically binds a soluble CD146 protein (sCD146), wherein said antibody comprises a light chain variable region (VL) comprising the sequence of SEQ ID NO: 1 and a heavy chain variable region (VH) comprising the sequence of SEQ ID NO: 2.

17. A multispecific antibody comprising an antigen binding region that specifically binds a sCD146 protein and an antigen binding region that specifically binds a VEGF protein, said multispecific antibody simultaneously binding the sCD146 and VEGF proteins.

18. The multispecific antibody of claim 17, wherein the multispecific antibody comprises an antigen binding region that specifically binds a VEGF protein and which comprises a light chain variable region (L) comprising one or more of the following CDRs:
H-CDR1: (SEQ ID NO: 23) or a variant thereof,
H-CDR2: (SEQ ID NO: 24) or a variant thereof, and
H-CDR3: (SEQ ID NO: 25) or a variant thereof,
and/or a heavy chain variable region (H) comprising one or more of the following CDRs:
L-CDR1: (SEQ ID NO: 26) or a variant thereof,
L-CDR2: (SEQ ID NO: 27) or a variant thereof, and
L-CDR3: (SEQ ID NO: 28) or a variant thereof,
said variants having at least 80% sequence identity to the recited CDR sequences.

19. The multispecific antibody of claim 18, wherein the multispecific antibody comprises a light chain variable region (VL) comprising sequence SEQ ID NO: 37 or a variant thereof, and a heavy chain variable region (VH) comprising sequence SEQ ID NO: 38, or a variant thereof, said variants having at least 80% sequence identity to the recited sequences.

20. The multispecific antibody of claim 17, wherein said multispecific antibody comprises an antigen binding region that specifically binds a sCD146 protein and which comprises a light chain variable region (L) comprising one or more of the following CDRs:
H-CDR1: (SEQ ID NO: 3) or a variant thereof,
H-CDR2: (SEQ ID NO: 4) or a variant thereof, and
H-CDR3: (SEQ ID NO: 5) or a variant thereof,
and/or a heavy chain variable region (H) comprising one or more of the following CDRs:
L-CDR1: (SEQ ID NO: 6) or a variant thereof,
L-CDR2: (SEQ ID NO: 7) or a variant thereof, and
L-CDR3: (SEQ ID NO: 8) or a variant thereof
said variants having at least 80% sequence identity to the recited CDR sequences.

21. The multispecific antibody of claim 20, wherein the multispecific antibody comprises a light chain variable region (VL) comprising the sequence of SEQ ID NO: 1 or a variant thereof, and a heavy chain variable region (VH) comprising the sequence of SEQ ID NO: 2 or a variant thereof, said variants having at least 80% sequence identity to the recited sequences.

22. A nucleic acid molecule or set of nucleic acid molecules encoding:
i) an antibody comprising an antigen binding region that specifically binds a soluble CD146 protein (sCD146), wherein said antibody comprises a light chain variable region (VL) comprising the sequence of SEQ ID NO: 1 and a heavy chain variable region (VH) comprising the sequence of SEQ ID NO: 2; or
ii) a multispecific antibody comprising an antigen binding region that specifically binds a sCD146 protein and an antigen binding region that specifically binds a VEGF protein, said multispecific antibody simultaneously binding the sCD146 and VEGF proteins, or
iii) a combination of:
a) an antibody comprising an antigen binding region that specifically binds a soluble CD146 protein (sCD146), wherein said antibody comprises a light chain variable region (VL) comprising the sequence of SEQ ID NO: 1 or a variant thereof and a heavy chain variable region (VH) comprising the sequence of SEQ ID NO: 2 or a variant thereof, an antibody comprising one or more of the CDRs of sequence SEQ ID NO: 3, 4, 5, 6, 7, or 8 or variant(s) thereof, with
b) an antibody comprising one or more of the CDRs of sequence SEQ ID NO: 23, 24, 25, 26, 27, or 28 or variant(s) thereof, an antibody comprising the VL comprising sequence SEQ ID NO: 37 or a variant thereof and VH sequences comprising sequence SEQ ID NO: 38, or a variant thereof, or bevacizumab antibody; or sequence(s) complementary to said encoding sequence(s),
wherein said variants have at least 80% sequence identity to the recited CDR sequences.

23. A vector comprising a nucleic acid molecule or a set of nucleic acid molecules as defined in claim 22.

24. A host cell comprising a nucleic acid or set of nucleic acids of claim 22 or a vector comprising said nucleic acid or set of nucleic acids.

25. A pharmaceutical composition comprising a first antibody comprising an antigen binding region that specifically binds a sCD146 protein and second antibody comprising an antigen binding region that specifically binds a VEGF protein, wherein the second antibody comprises SEQ ID NO: 23 (H-CDR1), SEQ ID NO: 24 (H-CDR2), SEQ ID NO: 25 (H-CDR3), SEQ ID NO: 26 (L-CDR1), SEQ ID NO: 27 (L-CDR2) and SEQ ID NO: 28 (L-CDR3), and a pharmaceutically acceptable excipient.

26. A pharmaceutical composition comprising a multispecific antibody according to claim 17 and a pharmaceutically acceptable excipient.

27. A method of treating cancer comprising administering the antibody according to claim 16 to a subject in need of treatment.

28. The method according to claim 27, wherein the subject has a glioblastoma or a CD146+ glioblastoma.

29. The method according to claim 27, wherein the subject suffering from cancer has been, is or will be treated with bevacizumab or has been identified as being resistant to bevacizumab treatment.

30. A method of treating cancer comprising administering the antibody according to claim 17 to a subject in need of treatment.

31. The method according to claim 30, wherein the subject has a glioblastoma or a CD146+ glioblastoma.

32. The method according to claim 30, wherein the subject suffering from cancer has been, is or will be treated with bevacizumab or has been identified as being resistant to bevacizumab treatment.

33. An in vitro or ex vivo method for monitoring the response to a bevacizumab treatment of a subject suffering from cancer comprising determining the soluble CD146 protein level of expression in a biological sample of said subject at two or more time points during said bevacizumab treatment, wherein a higher soluble CD146 protein level of expression in a biological sample of the subject at a later time point, compared to a reference value obtained in a biological sample of the subject at an earlier time point, is indicative of a resistance of the subject to said bevacizumab treatment whereas an equal or lower soluble CD146 protein level is indicative of a response of the subject to said bevacizumab treatment.

34. An in vitro or ex vivo method for predicting the response to a bevacizumab treatment of a subject suffering from cancer comprising i) determining the soluble CD146 protein expression level in a biological sample of the subject before bevacizumab treatment, ii) comparing the expression level determined at step i) with a reference value, and iii) concluding that the subject is likely to respond to bevacizumab treatment when the level determined at step i) is lower than the reference value, or concluding that the patient is unlikely to respond to bevacizumab treatment when the level determined at step i) is higher than the reference value.


SEQUENCE LISTING (63 sequences)

SEQ ID NO: 1 - protein, 107 aa, synthetic construct
EIVLTQSPDFQSVTPKEKVTITCRASQGISTSIYWYQQKPDQSPKLLIKFASQSISGVPSRFSGSGSGTDFTLTINRVEAEDAATYYCQQSYNLPYTFGAGTKLEIK

SEQ ID NO: 2 - protein, 114 aa, synthetic construct
QVKLVESGGGVVQPGRSLTLSCAASGFTFSDYGMHWIRQAPGKGLEWIAMIYYDSSKMYYADTVKGRFTISRDNSKNTLYLQMNSLRAEDTAVYYCAAFQFDYWGQGTMVTVSS

SEQ ID NO: 3 - protein, 5 aa, synthetic construct
DYGMH

SEQ ID NO: 4 - protein, 17 aa, synthetic construct
MIYYDSSKMYYADTVKG

SEQ ID NO: 5 - protein, 5 aa, synthetic construct
FQFDY

SEQ ID NO: 6 - protein, 10 aa, synthetic construct
ASQGISTSIY

SEQ ID NO: 7 - protein, 7 aa, synthetic construct
FASQSIS

SEQ ID NO: 8 - protein, 9 aa, synthetic construct
QQSYNLPYT

SEQ ID NO: 9 - protein, 30 aa, synthetic construct
QVKLVESGGGVVQPGRSLTLSCAASGFTFS

SEQ ID NO: 10 - protein, 14 aa, synthetic construct
WIRQAPGKGLEWIA

SEQ ID NO: 11 - protein, 32 aa, synthetic construct
RFTISRDNSKNTLYLQMNSLRAEDTAVYYCAA

SEQ ID NO: 12 - protein, 11 aa, synthetic construct
WGQGTMVTVSS

SEQ ID NO: 13 - protein, 24 aa, synthetic construct
EIVLTQSPDFQSVTPKEKVTITCR

SEQ ID NO: 14 - protein, 15 aa, synthetic construct
WYQQKPDQSPKLLIK

SEQ ID NO: 15 - protein, 32 aa, synthetic construct
GVPSRFSGSGSGTDFTLTINRVEAEDAATYYC

SEQ ID NO: 16 - protein, 10 aa, synthetic construct
FGAGTKLEIK

SEQ ID NO: 17 - protein, 107 aa, synthetic construct [sequence omitted]

SEQ ID NO: 18 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 214 aa, synthetic construct
EIVLTQSPDFQSVTPKEKVTITCRASQGISTSIYWYQQKPDQSPKLLIKFASQSISGVPSRFSGSGSGTDFTLTINRVEAEDAATYYCQQSYNLPYTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 444 aa, synthetic construct
QVKLVESGGGVVQPGRSLTLSCAASGFTFSDYGMHWIRQAPGKGLEWIAMIYYDSSKMYYADTVKGRFTISRDNSKNTLYLQMNSLRAEDTAVYYCAAFQFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 645 bp, synthetic construct
  source 1..645: mol_type = other DNA
gagattgtgcttactcagtctcctgacttccagtccgtgacaccgaaagagaaggtcaccatcacttgcagagcttcacaagggatttccaccagcatctactggtatcagcagaaacccgatcaatccccaaagttgctgatcaagttcgcctcacagagcataagcggagtaccttctcggttttctggcagtggaagtgggactgacttcacactgaccattaacagggttgaagcggaagatgcagccacctattactgtcagcagagctacaatctgccctatacgtttggtgctggcacaaagctcgagatcaaacgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgctga

SEQ ID NO: 22 - DNA, 1335 bp, synthetic construct
  source 1..1335: mol_type = other DNA
caggtgaagcttgtcgaatcaggtggaggtgtggttcaacccggaaggtcactgacactgtcttgtgccgctagtggcttcaccttcagcgactatggcatgcactggattcgacaggcacctgggaaagggttggagtggatagcgatgatctactacgactcctccaagatgtactacgccgatacggtcaaagggcggtttaccatcagtcgcgataacagcaagaataccctgtatctgcagatgaactctctcagagcagaggatactgccgtgtactattgcgctgcctttcagttcgactattggggacaaggcactatggtgacagtaagctccgcgagcaccaagggcccaagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagagagtggagcccaagagctgtgacaagacccacacctgccccccctgcccagcccccgagctgctgggcggacccagcgtgttcctgttcccccccaagcccaaggacaccctgatgatcagcagaacccccgaggtgacctgtgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggcaagtga

SEQ ID NO: 23 - protein, 9 aa, synthetic construct
GYTFNYFMN

SEQ ID NO: 24 - protein, 17 aa, synthetic construct
WINTYTGEPTYAADFKR

SEQ ID NO: 25 - protein, 14 aa, synthetic construct
YPHYYGSSHWYFDV

SEQ ID NO: 26 - protein, 11 aa, synthetic construct
SASQDISNYLN

SEQ ID NO: 27 - protein, 7 aa, synthetic construct
FTSSLHS

SEQ ID NO: 28 - protein, 9 aa, synthetic construct
QQYSTVPWT

SEQ ID NO: 29 - protein, 25 aa, synthetic construct [sequence omitted]

SEQ ID NO: 30 - protein, 14 aa, synthetic construct
WVRQAPGKGLEWVG

SEQ ID NO: 31 - protein, 32 aa, synthetic construct
RFTFSLDTSKSTAYLQMNSLRAEDTAVYYCAK

SEQ ID NO: 32 - protein, 11 aa, synthetic construct
WGQGTLVTVSS

SEQ ID NO: 33 - protein, 23 aa, synthetic construct
DIQMTQSPSSLSASVGDRVTITC

SEQ ID NO: 34 - protein, 15 aa, synthetic construct
WYQQKPGKAPKVLIY

SEQ ID NO: 35 - protein, 32 aa, synthetic construct
GVPSRFSGSGSGTDFTLTISSLQPEDFATYYC

SEQ ID NO: 36 - protein, 11 aa, synthetic construct
FGQGTKVEIKR

SEQ ID NO: 37 - protein, 108 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 38 - protein, 123 aa, synthetic construct [sequence omitted]

SEQ ID NO: 39 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 44 - DNA, 25 bp, synthetic construct
  source 1..25: mol_type = other DNA
caccgtcaactaccggctgcccagt

SEQ ID NO: 45 - DNA, 25 bp, synthetic construct
  source 1..25: mol_type = other DNA
aaacactgggcagccggtagttgac

SEQ ID NO: 46 - DNA, 25 bp, synthetic construct
  source 1..25: mol_type = other DNA
caccgaggcggacgagatgcgagcg

SEQ ID NO: 47 - DNA, 25 bp, synthetic construct
  source 1..25: mol_type = other DNA
aaaccgctcgcatctcgtccgcctc

SEQ ID NO: 48 - DNA, 25 bp, synthetic construct
  source 1..25: mol_type = other DNA
caccgtgctgctggccgtcgccctg

SEQ ID NO: 49 - DNA, 25 bp, synthetic construct
  source 1..25: mol_type = other DNA
aaaccagggcgacggccagcagcac

SEQ ID NO: 50 - DNA, 1656 bp, Homo sapiens
  source 1..1656: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctg

SEQ ID NO: 51 - DNA, 1659 bp, Homo sapiens
  source 1..1659: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccg

SEQ ID NO: 52 - DNA, 1662 bp, Homo sapiens
  source 1..1662: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggag

SEQ ID NO: 53 - DNA, 1665 bp, Homo sapiens
  source 1..1665: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccg

SEQ ID NO: 54 - DNA, 1668 bp, Homo sapiens
  source 1..1668: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggag

SEQ ID NO: 55 - DNA, 1671 bp, Homo sapiens
  source 1..1671: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagc

SEQ ID NO: 56 - DNA, 1674 bp, Homo sapiens
  source 1..1674: mol_type = genomic DNA
atggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccgg

SEQ ID NO: 57 - protein, 552 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKL

SEQ ID NO: 58 - protein, 553 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLP

SEQ ID NO: 59 - protein, 554 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPE

SEQ ID NO: 60 - protein, 555 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEP

SEQ ID NO: 61 - protein, 556 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPE

SEQ ID NO: 62 - protein, 557 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPES

SEQ ID NO: 63 - protein, 558 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESR